(19) United States
(12) Patent Application Publication
(10) Pub. No.: US 2006/0009487 A1
(43) Pub. Date: Jan. 12, 2006

(54) Heterocyclic derivatives for treatment of hyperlipidemia and related diseases

(76) Inventors: Jagadish C. Sircar, San Diego, CA (US); Richard J. Thomas, San Diego, CA (US); Haripada Khatuya, San Diego, CA (US); Igor Nikoulin, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/149,067

(22) Filed: Jun. 9, 2005

Related U.S. Application Data
(60) Provisional application No. 60/578,227, filed on Jun. 9, 2004.

(51) Int. Cl. C07D 215/12; A61K 31/47

(52) U.S. Cl. 514/311; 546/176

(57) ABSTRACT

The present invention provides compositions adapted to enhance reverse cholesterol transport in mammals. The compositions are suitable for oral delivery and useful in the treatment and/or prevention of hypercholesterolemia, atherosclerosis and associated cardiovascular diseases.

### CROSS-REFERENCE TO RELATED APPLICATION

This application claims priority under 35 U.S.C. § 119(e) to U.S. Provisional Application No. 60/578,227, filed Jun. 9, 2004, which is incorporated herein by reference.

### BACKGROUND OF THE INVENTION

1. Field of the Invention

The invention relates to small molecule mediators of reverse cholesterol transport (RCT) for treating hypercholesterolemia and associated cardiovascular diseases and other diseases.

2. Description of the Related Art

It is now well-established that elevated serum cholesterol (“hypercholesterolemia”) is a causal factor in the develoment of atherosclerosis, a progressive accumulation of cholesterol within the arterial walls. Hypercholesterolemia and atherosclerosis are leading causes of cardiovascular diseases, including hypertension, coronary artery disease, heart attack and stroke. About 1.1 million individuals suffer from heart attack each year in the United States alone, the costs of which are estimated to exceed $117 billion. Although there are numerous pharmaceutical strategies for lowering cholesterol levels in the blood, many of these have undesirable side effects and have raised safety concerns. Moreover, none of the commercially available drug therapies adequately stimulate reverse cholesterol transport, an important metabolic pathway that removes cholesterol from the body.

Circulating cholesterol is carried by plasma lipoproteins—particles of complex lipid and protein composition that transport lipids in the blood. Low density lipoproteins (LDLs), and high density lipoproteins (HDLs) are the major cholesterol carriers. LDLs are believed to be responsible for the delivery of cholesterol from the liver (where it is synthesized or obtained from dietary sources) to extrahepatic tissues in the body. The term “reverse cholesterol transport” describes the transport of cholesterol from extrahepatic tissues to the liver where it is catabolized and eliminated. It is believed that plasma HDL particles play a major role in the reverse transport process, acting as scavengers of tissue cholesterol.

Compelling evidence supports the concept that lipids deposited in atherosclerotic lesions are derived primarily from plasma LDL; thus, LDLs have popularly become known as the “bad” cholesterol. In contrast, plasma HDL levels correlate inversely with coronary heart disease—indeed, high plasma levels of HDL are regarded as a negative risk factor. It is hypothesized that high levels of plasma HDL are not only protective against coronary artery disease, but may actually induce regression of atherosclerotic plaques (e.g. see Badimon et al., 1992, Circulation 86 (Suppl. III)86-94). Thus, HDLs have popularly become known as the “good” cholesterol.

The amount of intracellular cholesterol liberated from the LDLs controls cellular cholesterol metabolism. The accumulation of cellular cholesterol derived from LDLs controls three processes: (1) it reduces cellular cholesterol synthesis by turning off the synthesis of HMGCoA reductase, a key enzyme in the cholesterol biosynthetic pathway; (2) the incoming LDL-derived cholesterol promotes storage of cholesterol by activating LCAT, the cellular enzyme which converts cholesterol into cholesteryl esters that are deposited in storage droplets; and (3) the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading. (For a review, see Brown & Goldstein, In: The Pharmacological Basis Of Therapeutics, 8th Ed., Goodman & Gilman, Pergamon Press, New York, 1990, Ch. 36, pp. 874-896).

Reverse cholesterol transport (RCT) is the pathway by which peripheral cell cholesterol can be returned to the liver for recycling to extrahepatic tissues, or excreted into the intestine as bile. The RCT pathway represents the only means of eliminating cholesterol from most extrahepatic tissues. The RCT consists mainly of three steps: (1) cholesterol efflux, the initial removal of cholesterol from peripheral cells; (2) cholesterol esterification by the action of lecithin:cholesterol acyltransferase (LCAT), preventing a re-entry of effluxed cholesterol into the peripheral cells; and (3) uptake/delivery of HDL cholesteryl ester to liver cells. LCAT is the key enzyme in the RCT pathway and is produced mainly in the liver and circulates in plasma associated with the HDL fraction. LCAT converts cell derived cholesterol to cholesteryl esters which are sequestered in HDL destined for removal. The RCT pathway is mediated by HDLs.

HDL is a generic term for lipoprotein particles which are characterized by their high density. The main lipidic constituents of HDL complexes are various phospholipids, cholesterol (ester) and triglycerides. The most prominent apolipoprotein components are A-I and A-II which determine the functional characteristics of HDL.

Each HDL particle contains at least one copy (and usually two to four copies) of apolipoprotein A-1 (ApoA-I). ApoA-I is synthesized by the liver and small intestine as preproapolipoprotein which is secreted as a proprotein that is rapidly cleaved to generate a mature polypeptide having 243 amino acid residues. ApoA-I consists mainly of 6 to 8 different 22 amino acid repeats spaced by a linker moiety which is often proline, and in some cases consists of a stretch made up of several residues. ApoA-I forms three types of stable complexes with lipids: small, lipid-poor complexes referred to as pre-beta-1 HDL; flattened discoidal particles containing polar lipids (phospholipid and cholesterol) referred to as pre-beta-2 HDL; and spherical particles containing both polar and nonpolar lipids, referred to as spherical or mature HDL (HDL₃ and HDL₂). Although most HDL in circulation contains both ApoA-I and ApoA-II, the fraction of HDL which contains only ApoA-I (AI-HDL) appears to be more effective in RCT. Epidemiologic studies support the hypothesis that AI-HDL is anti-atherogenic. (Parra et al., 1992, Arterioscler. Thromb. 12:701-707; Decossin et al., 1997, Eur. J. Clin. Invest. 27:299-307).

Several lines of evidence based on data obtained in vivo implicate the HDL and its major protein component, ApoA-I, in the prevention of atherosclerotic lesions, and potentially, the regression of plaques—making these attractive targets for therapeutic intervention. First, an inverse correlation exists between serum ApoA-I (HDL) concentration and atherogenesis in man (Gordon & Rifkind, 1989, N. Eng. J. Med. 321:1311-1316; Gordon et al., 1989, Circulation 79:8-15). Indeed, specific subpopulations of HDL have been associated with a reduced risk for atherosclerosis in humans (Miller, 1987, Amer. Heart 113:589-597; Cheung et al., 1991, Lipid Res. 32:383-394); Fruchart & Ailhaud, 1992, Clin. Chem. 38:79).

Second, animal studies support the protective role of ApoA-I (HDL). Treatment of cholesterol fed rabbits with ApoA-I or HDL reduced the development and progression of plaque (fatty streaks) in cholesterol-fed rabbits (Koizumi et al., 1988, J. Lipid Res. 29:1405-1415; Badimon et al., 1989, Lab. Invest. 60:455-461; Badimon et al., 1990, J. Clin. Invest. 85:1234-1241). However, the efficacy varied depending upon the source of HDL (Beitz et al., 1992, Prostaglandins, Leukotrienes and Essential Fatty Acids 47:149-152; Mezdour et al., 1995, Atherosclerosis 113:237-246).

Third, direct evidence for the role of ApoA-I was obtained from experiments involving transgenic animals. The expression of the human gene for ApoA-I transferred to mice genetically predisposed to diet-induced atherosclerosis protected against the development of aortic lesions (Rubin et al., 1991, Nature 353:265-267). The ApoA-I transgene was also shown to suppress atherosclerosis in ApoE-deficient mice and in Apo(a) transgenic mice (Paszty et al., 1994, J. Clin. Invest. 94:899-903; Plump et al., 1994, PNAS. USA 91:9607-9611; Liu et al., 1994, J. Lipid Res. 35:2263-2266). Similar results were observed in transgenic rabbits expressing human ApoA-I (Duverger, 1996, Circulation 94:713-717; Duverger et al., 1996, Arterioscler. Thromb. Vasc. Biol. 16:1424-1429), and in transgenic rats where elevated levels of human ApoA-I protected against atherosclerosis and inhibited restenosis following balloon angioplasty (Burkey et al., 1992, Circulation, Supplement I, 86:1-472, Abstract No. 1876; Burkey et al., 1995, J. Lipid Res. 36:1463-1473).

Current Treatments for Hypercholesterolemia and other Dyslipidemias

In the past two decades or so, the segregation of cholesterolemic compounds into HDL and LDL regulators and recognition of the desirability of decreasing blood levels of LDL has led to the development of a number of drugs. However, many of these drugs have undesirable side effects and/or are contraindicated in certain patients, particularly when administered in combination with other drugs. These drugs and therapeutic strategies include:
(1) bile-acid-binding resins, which interrupt the recycling of bile acids from the intestine to the liver [e.g., cholestyramine (QUESTRAN LIGHT, Bristol-Myers Squibb), and colestipol hydrochloride (COLESTID, Pharmacia & Upjohn Company)];
(2) statins, which inhibit cholesterol synthesis by blocking HMGCoA reductase—the key enzyme involved in cholesterol biosynthesis [e.g., lovastatin (MEVACOR, Merck & Co., Inc.), a natural product derived from a strain of Aspergillus, pravastatin (PRAVACHOL, Bristol-Myers Squibb Co.), and atorvastatin (LIPITOR, Warner Lambert)];
(3) niacin is a water-soluble vitamin B-complex which diminishes production of VLDL and is effective at lowering LDL;
(4) fibrates are used to lower serum triglycerides by reducing the VLDL fraction and may in some patient populations give rise to modest reductions of plasma cholesterol via the same mechanism [e.g., clofibrate (ATROMID-S, Wyeth-Ayerst Laboratories), and gemfibrozil (LOPID, Parke-Davis)];
(5) estrogen replacement therapy may lower cholesterol levels in post-menopausal women;
(6) long chain alpha,omego-dicarboxylic acids have been reported to lower serum triglyceride and cholesterol (See, e.g., Bisgaier et al., 1998, J. Lipid Res. 39:17-30; WO 98/30530; U.S. Pat. No. 4,689,344; WO 99/00116; U.S. Pat. No. 5,756,344; U.S. Pat. No. 3,773,946; U.S. Pat. No. 4,689,344; U.S. Pat. No. 4,689,344; U.S. Pat. No. 4,689,344; and U.S. Pat. No. 3,930,024);
(7) other compounds including ethers (See, e.g., U.S. Pat. No. 4,711,896; U.S. Pat. No. 5,756,544; U.S. Pat. No. 6,506,799), phosphates of dolichol (U.S. Pat. No. 4,613,593), and azolidinedione derivatives (U.S. Pat. No. 4,287,200) are disclosed as lowering serum triglyceride and cholesterol levels.

None of these currently available drugs for lowering cholesterol safely elevate HDL levels and stimulate RCT. Indeed, most of these current treatment strategies appear to operate on the cholesterol transport pathway, modulating dietary intake, recycling, synthesis of cholesterol, and the VLDL population.

ApoA-I Azonists for Treatment of Hypercholesterolemia

In view of the potential role of HDL, i.e., both ApoA-I and its associated phospholipid, in the protection against atherosclerotic disease, human clinical trials utilizing recombinantly produced ApoA-I were commenced, discontinued and apparently re-commenced by UCB Belgium (Pharmaprojects, Oct. 27, 1995; IMS R&D Focus, Jun. 30, 1997; Drug Status Update, 1997, Atherosclerosis 2(6):261-265); see also M. Eriksson at Congress, “The Role of HDL in Disease Prevention,” Nov. 7-9, 1996, Fort Worth; Lacko & Miller, 1997, J. Lip. Res. 38:1267-1273; and WO 94/13819) and were commenced and discontinued by Bio-Tech (Pharmaprojects, Apr. 7, 1989). Trials were also attempted using ApoA-I to treat septic shock (Opal, “Reconstituted HDL as a Treatment Strategy for Sepsis,” IBC's 7th International Conference on Sepsis, Apr. 28-30, 1997, Washington, D.C.; Gouni et al., 1993, J. Lipid Res. 94:139-146; Levine, WO 96/04916). However, there are many pitfalls associated with the production and use of ApoA-I, making it less than ideal as a drug; e.g., ApoA-I is a large protein that is difficult and expensive to produce; significant manufacturing and reproducibility problems must be overcome with respect to stability during storage, delivery of an active product and half-life in vivo.

In view of these drawbacks, attempts have been made to prepare peptides that mimic ApoA-I. Since the key activities of ApoA-I have been attributed to the presence of multiple repeats of a unique secondary structural feature in the protein—a class A amphipathic α-helix (Segrest, 1974, FEBS Lett. 38:247-253; Segrest et al., 1990, PROTEINS: Structure, Function and Genetics 8:103-117), most efforts to design peptides which mimic the activity of ApoA-I have focused on designing peptides which form class A-type amphipathic α-helices (See e.g., Background discussions in U.S. Pat. Nos. 6,376,464 and 6,506,799; incorporated herein in their entirety by reference thereto).

In one study, Fukushima et al. synthesized a 22-residue peptide composed entirely of Glu, Lys and Leu residues arranged periodically so as to form an amphipathic α-helix with equal-hydrophilic and hydrophobic faces (“ELK peptide”) (Fukushima et al., 1979, J. Amer. Chem. Soc. 101(13):3703-3704; Fukushima et al., 1980, J. Biol. Chem. 255:10651-10657). The ELK peptide shares 41% sequence homology with the 198-219 fragment of ApoA-I. The ELK peptide was shown to effectively associate with phospholipids and mimic some of the physical and chemical properties of ApoA-I (Kaiser et al., 1983, PNAS USA 80:1137-1140; Kaiser et al., 1984, Science 223:249-255; Fukushima et al., 1980, supra; Nakagawa et al., 1985, J. Am. Chem. Soc. 107:7087-7092). A dimer of this 22-residue peptide was later found to more closely mimic ApoA-I than the monomer; based on these results, it was suggested that the 44-mer, which is punctuated in the middle by a helix breaker (either Gly or Pro), represented the minimal functional domain in ApoA-I (Nakagawa et al., 1985, supra).

Another study involved model amphipathic peptides called “LAP peptides” (Pownall et al., 1980, PNAS USA 77(6):3154-3158; Sparrow et al., 1981, In: Peptides: Synthesis-Structure-Function, Roch and Gross, Eds., Pierce Chem. Co., Rockford, Ill., 253-256). Based on lipid binding studies with fragments of native apolipoproteins, several LAP peptides were designed, named LAP-16, LAP-20 and LAP-24 (containing 16, 20 and 24 amino acid residues, respectively). These model amphipathic peptides share no sequence homology with the apolipoproteins and were designed to have hydrophilic faces organized in a manner unlike the class A-type amphipathic helical domains associated with apolipoproteins (Segrest et al., 1992, J. Lipid Res. 33:141-166). From these studies, the authors concluded that a minimal length of 20 residues is necessary to confer lipid-binding properties to model amphipathic peptides.

Studies with mutants of LAP20 containing a proline residue at different positions in the sequence indicated that a direct relationship exists between lipid binding and LCAT activation, but that the helical potential of a peptide alone does not lead to LCAT activation (Ponsin et al., 1986, J. Biol. Chem. 261(20):9202-9205). Moreover, the presence of this helix breaker (Pro) close to the middle of the peptide reduced its affinity for phospholipid surfaces as well as its ability to activate LCAT. While certain of the LAP peptides were shown to bind phospholipids (Sparrow et al., supra), controversy exists as to the extent to which LAP peptides are helical in the presence of lipids (Buchko et al., 1996, J. Biol. Chem. 271(6):3039-3045; Zhong et al., 1994, Peptide Research 7(2):99-106).

Segrest et al. have synthesized peptides composed of 18 to 24 amino acid residues that share no sequence homology with the helices of ApoA-I (Kannelis et al., 1980, J. Biol. Chem. 255(3):11464-11472; Segrest et al., 1983, J. Biol. Chem. 258:2290-2295). The sequences were specifically designed to mimic the amphipathic helical domains of class A exchangeable apolipoproteins in terms of hydrophobic moment (Eisenberg et al., 1982, Nature 299:371-374) and charge distribution (Segrest et al., 1990, Proteins 8:103-117; U.S. Pat. No.4,643,988). One 18-residue peptide, the “18A” peptide, was designed to be a model class-A α-helix (Segrest et al., 1990, supra). Studies with these peptides and other peptides having a reversed charged distribution, like the “18R” peptide, have consistently shown that charge distribution is critical for activity; peptides with a reversed charge distribution exhibit decreased lipid affinity relative to the 18A class-A mimics and a lower helical content in the presence of lipids (Kanellis et al., 1980, J. Biol. Chem. 255:11464-11472; Anantharamaiah et al., 1985, J. Biol. Chem. 260:10248-10255; Chung et al., 1985, J. Biol. Chem. 260:10256-10262; Epand et al., 1987, J. Biol. Chem. 262:9389-9396; Anantharamaiah et al., 1991, Adv. Exp. Med. Biol. 285:131-140).

A “consensus” peptide containing 22-amino acid residues based on the sequences of the helices of human ApoA-I has also been designed (Anantharamaiah et al., 1990, Arteriosclerosis 10(1):95-105; Venkatachalapathi et al., 1991, Mol. Conformation and Biol. Interactions, Indian Acad. Sci. B:585-596). The sequence was constructed by identifying the most prevalent residue at each position of the hypothesized helices of human ApoA-I. Like the peptides described above, the helix formed by this peptide has positively charged amino acid residues clustered at the hydrophilic-hydrophobic interface, negatively charged amino acid residues clustered at the center of the hydrophilic face and a hydrophobic angle of less than 180°. While a dimer of this peptide is somewhat effective in activating LCAT, the monomer exhibited poor lipid binding properties (Venkatachalapathi et al., 1991, supra).

Based primarily on in vitro studies with the peptides described above, a set of “rules” has emerged for designing peptides which mimic the function of ApoA-I. Significantly, it is thought that an amphipathic α-helix having positively charged residues clustered at the hydrophilic-hydrophobic interface and negatively charged amino acid residues clustered at the center of the hydrophilic face is required for lipid affinity and LCAT activation (Venkatachalapathi et al., 1991, supra). Anantharamaiah et al. have also indicated that the negatively charged Glu residue at position 13 of the consensus 22-mer peptide, which is positioned within the hydrophobic face of the α-helix, plays an important role in LCAT activation (Anantharamaiah et al., 1991, supra). Furthermore, Brasseur has indicated that a hydrophobic angle (pho angle) of less than 180° is required for optimal lipid-apolipoprotein complex stability, and also accounts for the formation of discoidal particles having the peptides around the edge of the lipid bilayer (Brasseur, 1991, J. Biol. Chem. 66(24):16120-16127). Rosseneu et al. have also insisted that a hydrophobic angle of less than 180° is required for LCAT activation (WO 93/25581).

However, despite the progress in elucidating “rules” for designing ApoA-I agonists, to date the best ApoA-I agonists are reported as having less than 40% of the activity of intact ApoA-I. None of the peptide agonists described in the literature have been demonstrated to be useful as a drug. Thus, there is a need for the development of a stable molecule that mimics the activity of ApoA-I and which is relatively simple and cost-effective to produce. Preferably, candidate molecules would mediate both indirect and direct RCT. Such molecules would be smaller than existing peptide agonists, and have broader functional spectra. However, the “rules” for designing efficacious mediators of RCT have not been fully elucidated and the principles for designing organic molecules with the function of ApoA-I are unknown.

### SUMMARY OF THE INVENTION

A mediator of reverse cholesterol transport is disclosed comprising the structure:

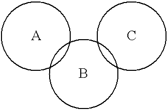

wherein A, B, and C may be in any order, and wherein:

A comprises an acidic moiety, comprising an acidic group or a bioisostere thereof;

B comprises an aromatic or lipophilic moiety comprising at least a portion of HMG CoA reductase inhibitor or analog thereof; and

C comprises a basic moiety, comprising a basic group or a bioisostere thereof.

Preferably, at least one of the alpha amino or alpha carboxy groups have been removed from their respective amino or carboxy terminal moieties.

If not removed, the alpha amino group is preferably capped with a protecting group selected from the group consisting of acetyl, phenylacetyl, benzoyl, pivolyl, 9-fluorenylmethyloxycarbonyl, 2-napthylic acid, nicotinic acid, a CH₃—(CH₂)ₙ—CO— where n ranges from 3 to 20, di-tert-butyl-4-hydroxy-phenyl, naphthyl, substituted naphthyl, Fmoc, biphenyl, substituted phenyl, substituted heterocycles, alkyl, aryl, substituted aryl, cycloalkyl, fused cycloalkyl, saturated heteroaryl, and substituted saturated heteroaryl.

If not removed, the alpha carboxy group is preferably capped with a protecting group selected from the group consisting of an amine, such as RNH where R═H, di-tert-butyl-4-hydroxy-phenyl, naphthyl, substituted naphthyl, Fmoc, biphenyl, substituted phenyl, substituted heterocycles, alkyl, aryl, substituted aryl, cycloalkyl, fused cycloalkyl, saturated heteroaryl, and substituted saturated heteroaryl.

Bioisosteres of the acidic group may be selected from the group consisting of: 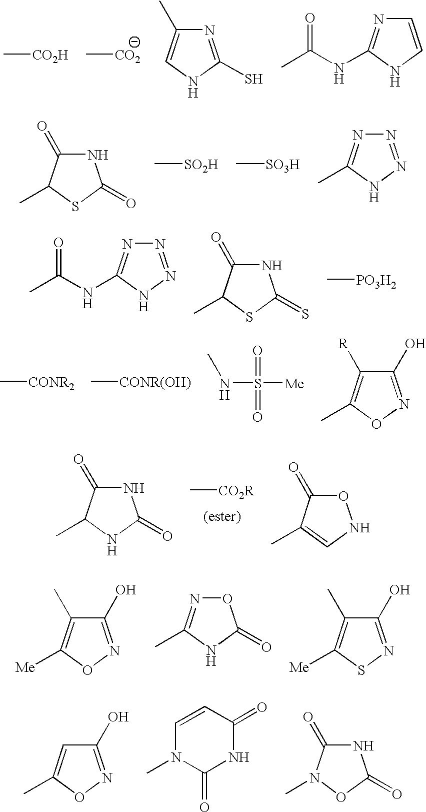
Bioisosteres of the basic group may be selected from the group consisting of:

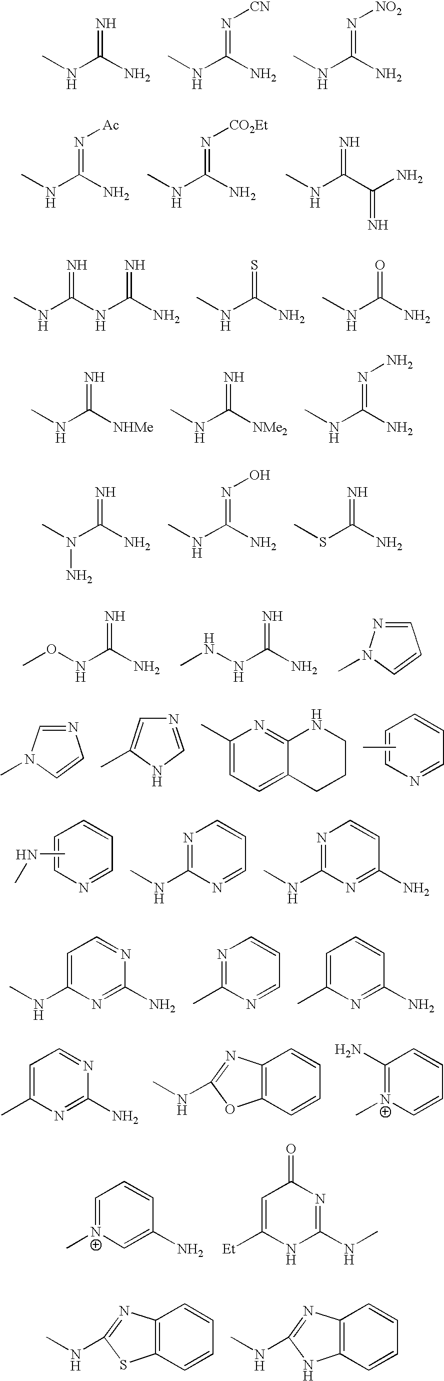

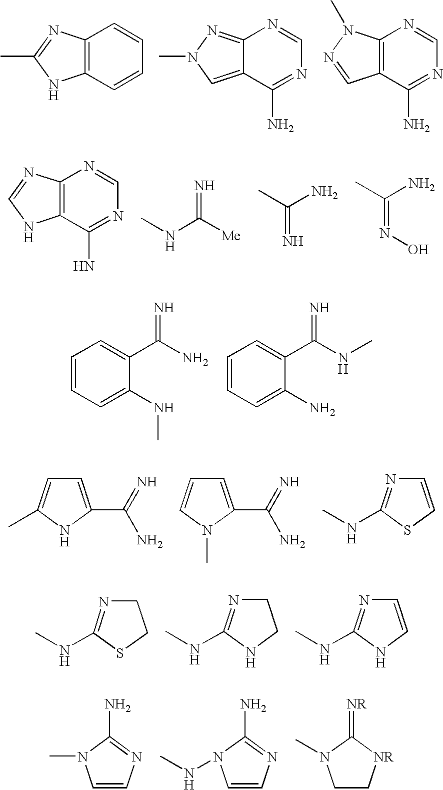

The following mediators are disclosed in accordance with preferred embodiments:

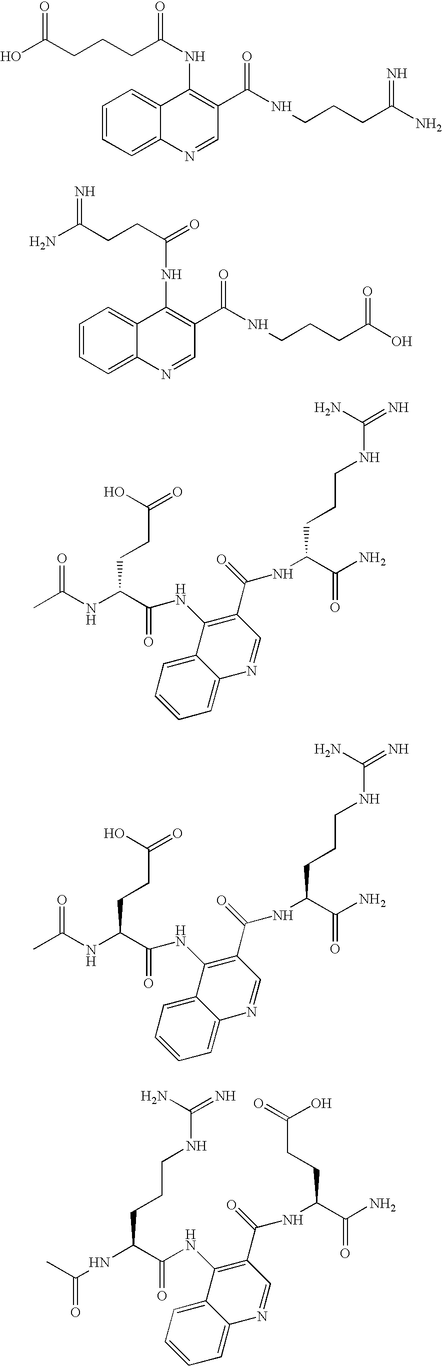

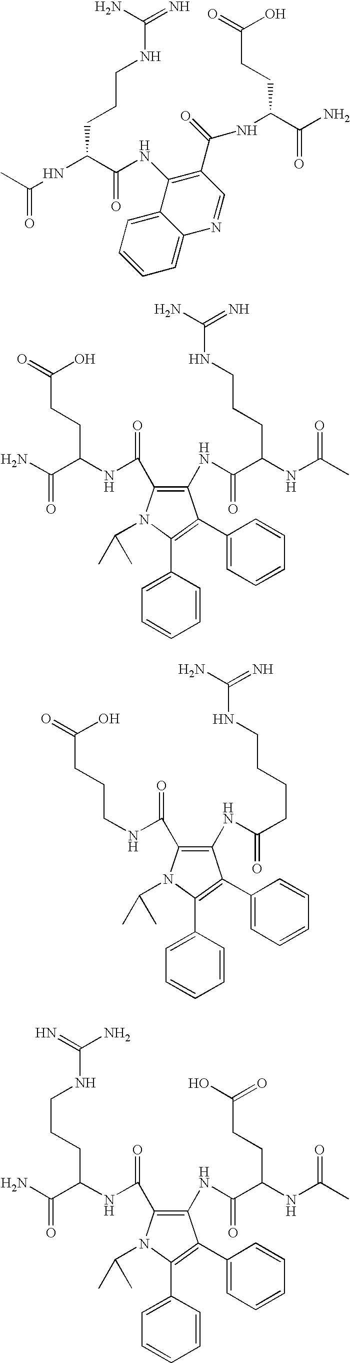

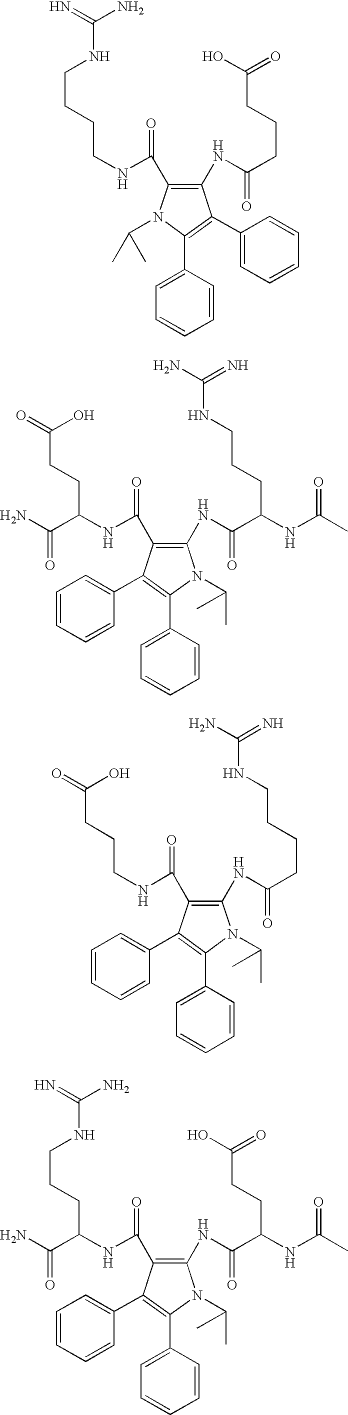

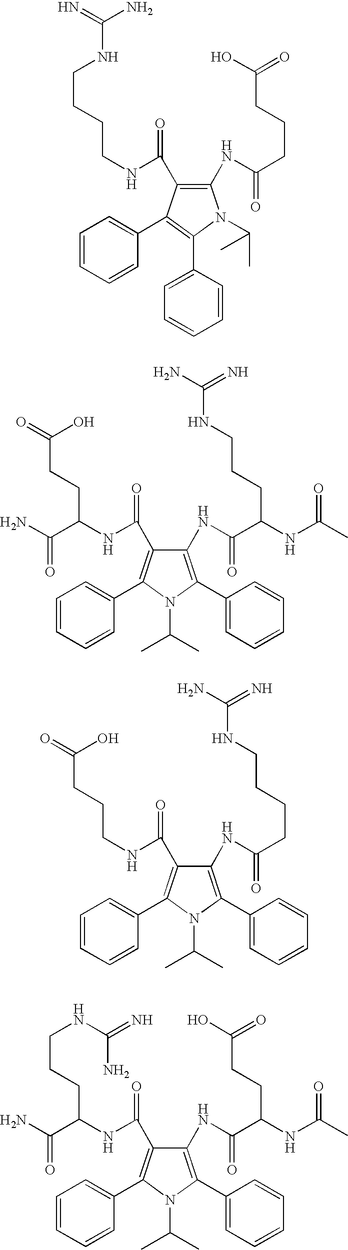

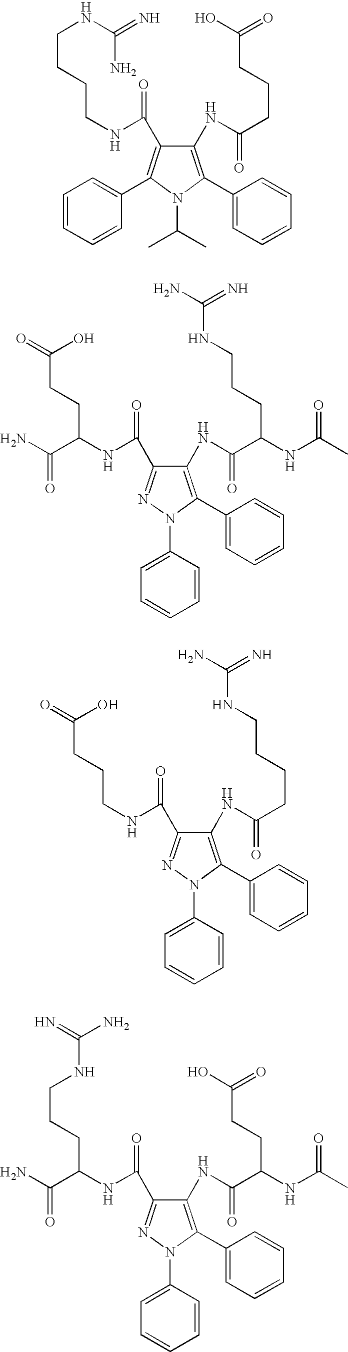

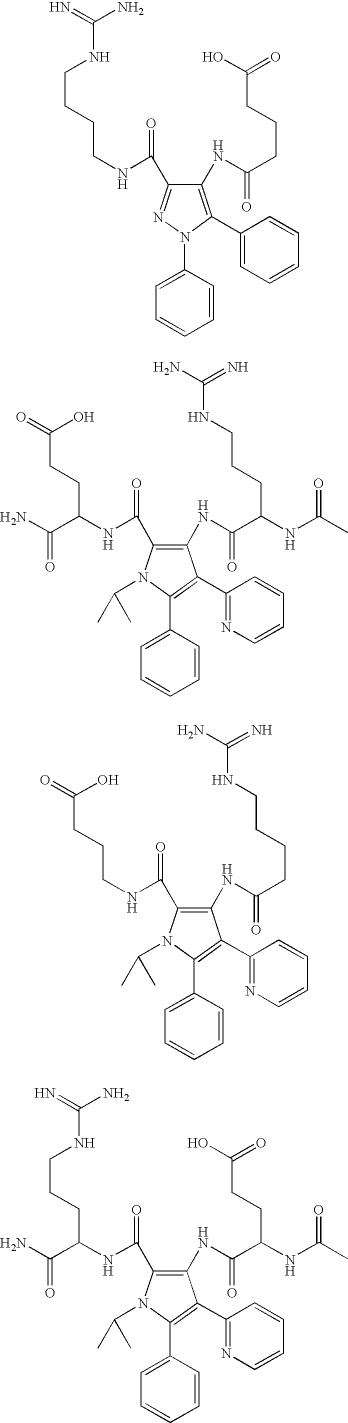

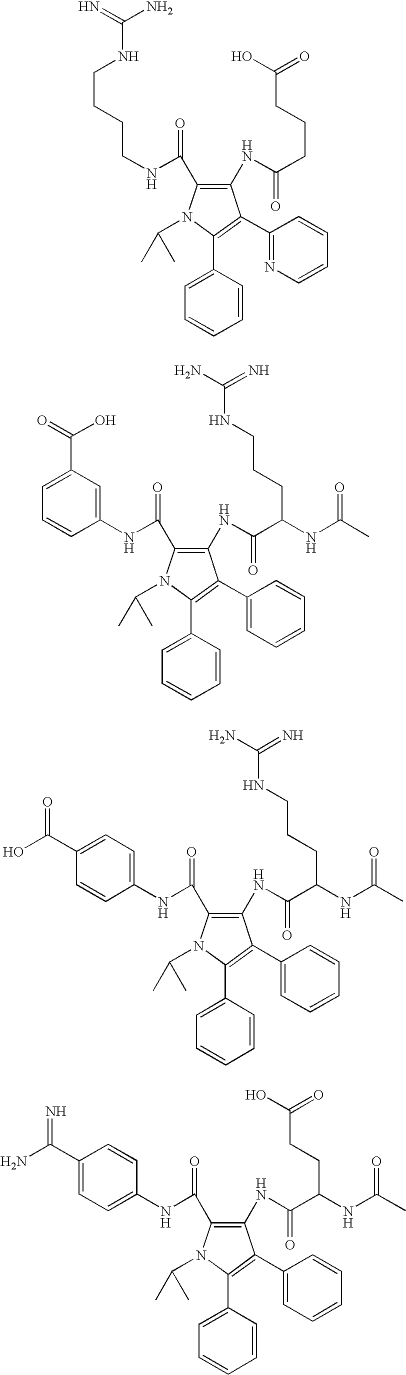

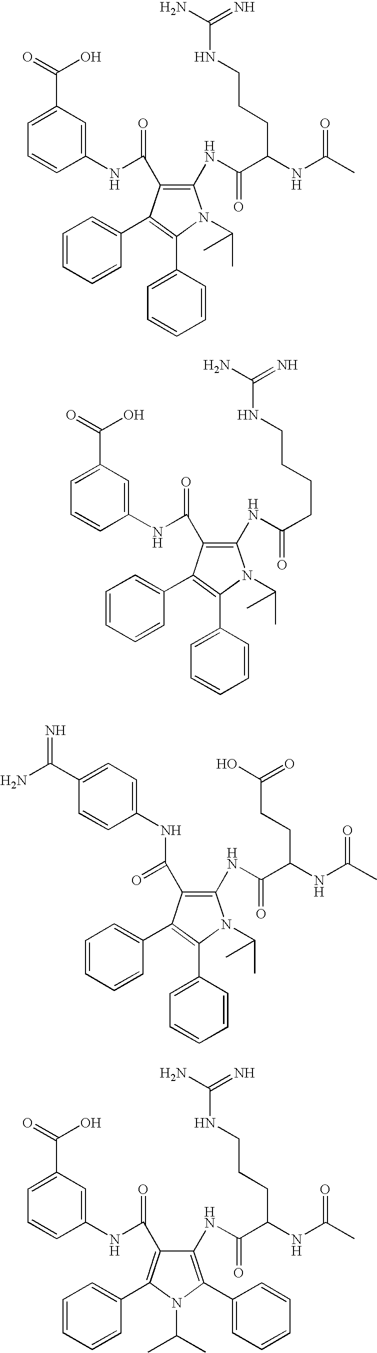

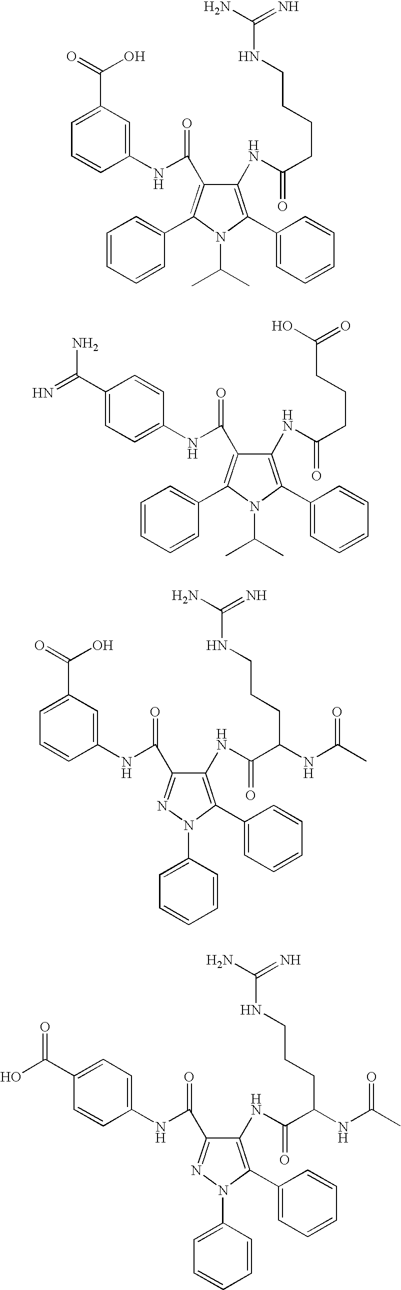

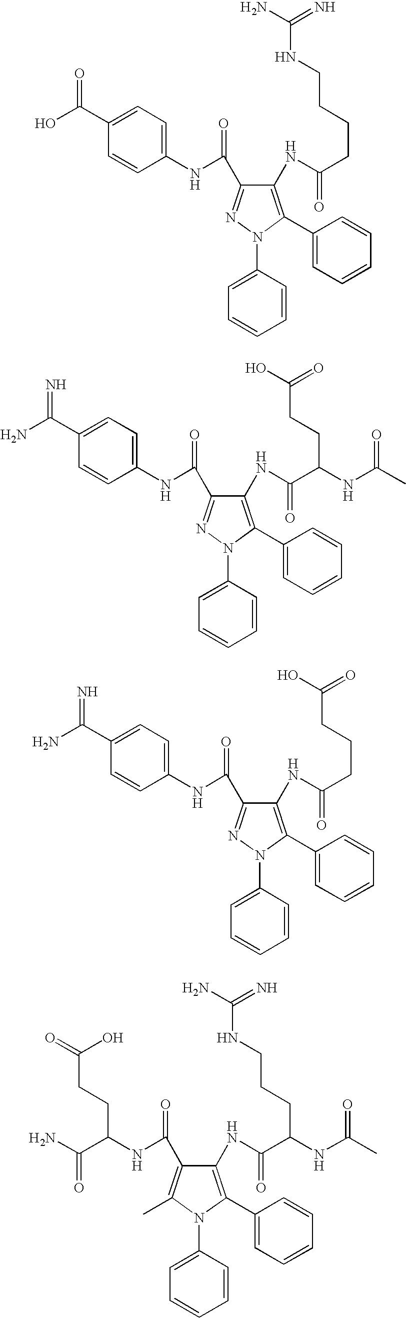

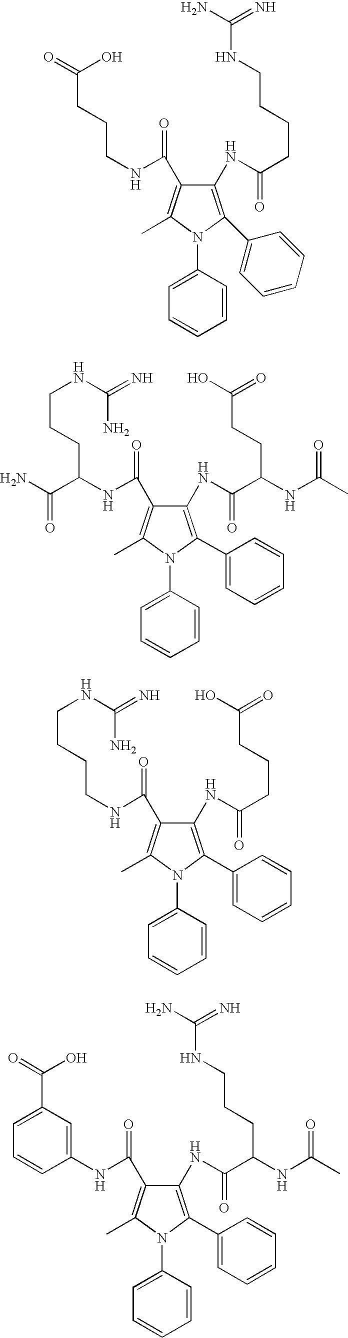

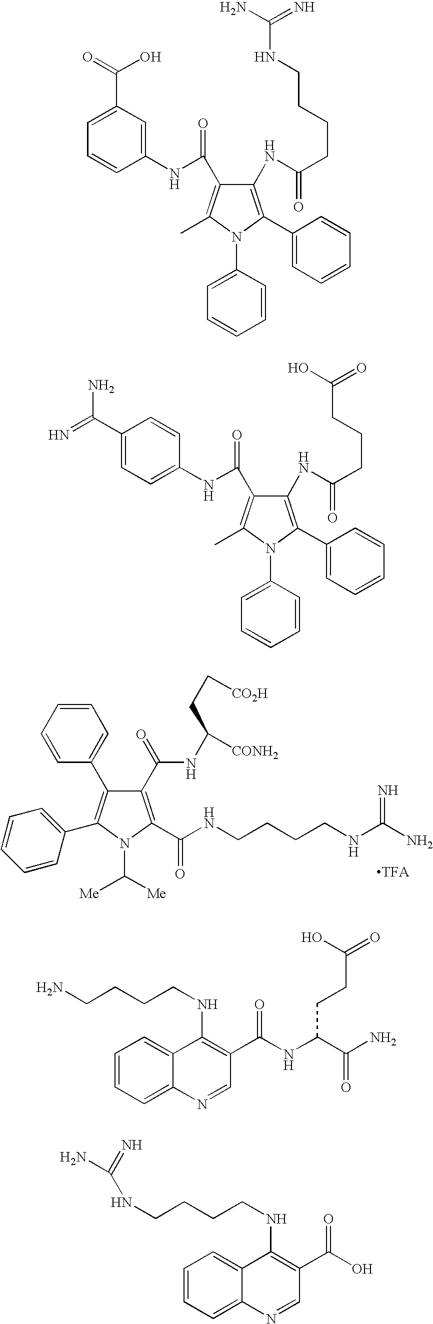

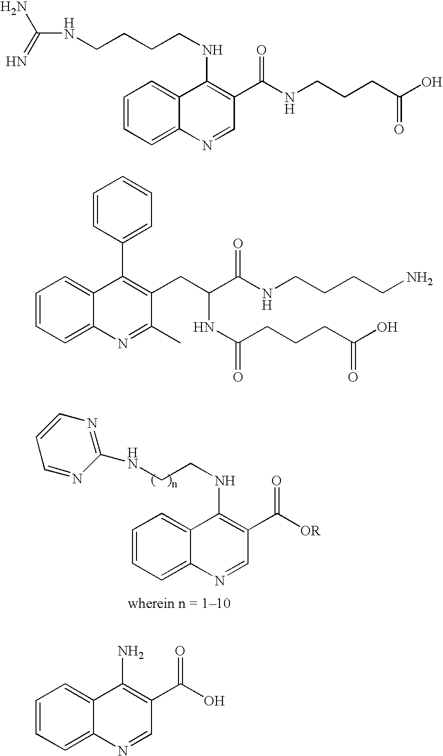

In preferred embodiments, the following compounds are disclosed: 4-Agmatine-3-amidoGABAquinoline, 4-(1-(4-aminobutylcarbamoyl)-2-(2-methyl-4-phenylquinolin-3-yl)ethylcarbamoyl)butanoic acid, and 4-(5-guanidinopentylamino)quinoline-3-carboxylic acid. Any underivatized amino and/or carboxy terminal amino acid residues in the above list of preferred compounds are capped with a protecting group. In another preferred embodiment, the mediator has the structure:

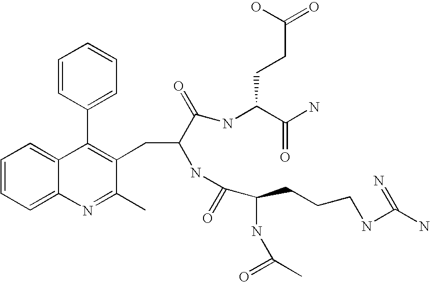

### DETAILED DESCRIPTION OF THE PREFERRED EMBODIMENT

The mediators of RCT in preferred embodiments of the invention mimic ApoA-I function and activity. In a broad aspect, these mediators are molecules comprising three regions, an acidic region, a lipophilic (e.g., aromatic) region, and a basic region. The molecules preferably contain a positively charged region, a negatively charged region, and an uncharged, lipophilic region. The locations of the regions with respect to one another can vary between molecules; thus, in a preferred embodiment, the molecules mediate RCT regardless of the relative positions of the three regions within each molecule. Whereas in some preferred embodiments, the molecular template or model comprises an “acidic” amino acid-derived residue, a lipophilic moiety, and a basic amino acid-derived residue, linked in any order to form a mediator of RCT, in other preferred embodiments, the molecular model can be embodied by a single residue having acidic, lipophilic and basic regions, such as for example, the amino acid, phenylalanine.

In some preferred embodiments, the molecular mediators of RCT comprise natural L- or D- amino acids, amino acid analogs (synthetic or semisynthetic), and amino acid derivatives. For example, the mediator may include an “acidic” amino acid residue or analog thereof, an aromatic or lipophilic scaffold, and a basic amino acid residue or analog thereof, the residues being joined by peptide or amide bond linkages, or any other bonds. The molecular mediators of RCT share the common aspect of reducing serum cholesterol through enhancing direct and/or indirect RCT pathways (i.e., increasing cholesterol efflux), ability to activate LCAT, and ability to increase serum HDL concentration.

In a preferred embodiment, the mediator of reverse cholesterol transport preferably comprises an acid group, a lipophilic group and a basic group, and comprises the sequence: X1-X2-X3, X1-X2-Y3, Y1-X2-X3, or Y1-X2-Y3 wherein: X1 is an acidic amino acid or analog thereof; X2 is an aromatic or a lipophilic portion of a HMG CoA reductase inhibitor (e.g., a scaffold or pharmacophore); X3 is a basic amino acid or analog thereof, Y1 is an acidic amino acid analog without the alpha amino group; and Y3 is a basic amino acid analog without the alpha carboxy group. When the amino terminal alpha amino group is present (e.g., X1), it further comprises a first protecting group, and when the carboxy terminal alpha carboxy group is present (e.g., X3), it further comprises a second protecting group. The first (amino terminal) protecting groups are preferably selected from the group consisting of an acetyl, phenylacetyl, pivolyl, 2-napthylic acid, nicotinic acid, a CH₃—(CH₂)ₙ—CO— where n ranges from 1 to 20, and an amide of acetyl, phenylacetyl, di-tert- butyl-4-hydroxy-phenyl, naphthyl, substituted naphthyl, FMOC, biphenyl, substituted phenyl, substituted heterocycles, alkyl, aryl, substituted aryl, cycloalkyl, fused cycloalkyl, saturated heteroaryl, substituted saturated heteroaryl and the like. The second (carboxy terminal) protecting groups are preferably selected from the group consisting of an amine such as RNH₂ where R═di-tert-butyl-4-hydroxy-phenyl, naphthyl, substituted naphthyl, FMOC, biphenyl, substituted phenyl, substituted heterocycles, alkyl, aryl, substituted aryl, cycloalkyl, fused cycloalkyl, saturated heteroaryl, substituted saturated heteroaryl and the like. The order of the acidic, lipophilic and basic groups can be scrambled in any and all possible ways to provide compounds that retain the basic features of the molecular model. In some preferred embodiments, analogs of X1 and X3 may comprise bioisosteres of the acid and base R groups. In other embodiments, one or more of X1, X2 or X3 are D or other modified synthetic amino acid residues to provide metabolically stable molecules. This could also be achieved by peptidomimetic approach i.e. reversing the peptide bonds in the backbone or similar groups.

In another embodiment, the mediator can be incorporated into a larger entity, such as a peptide of about 1 to 10 amino acids, or a molecule.

A scaffold is used herein to denote a pharmacophore which is a model to simplify an interaction process between a ligand (candidate drug molecule) and a protein. A scaffold can possess certain features of the native molecule fixed in an active site of the protein. It can be assumed that these features interact with some complementary features in the cavity of the protein. Variations can be derived by attaching functional groups to the scaffold. Preferably, we define a scaffold by the following heuristic: A scaffold is a mimic of at least a portion of an HMG CoA reductase inhibitor that is lipophilic or aromatic.

The terms “bioisostere”, “bioisosteric replacement”, “bioisosterism” and closely related terms as used herein have the same meanings as those generally recognized in the art. Bioisosteres are atoms, ions, or molecules in which the peripheral layers of electrons can be considered substantially similar. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Bioisosterism arises from a reasonable expectation that a proposed bioisosteric replacement will result in maintenance of similar biological properties. Such a reasonable expectation may be based on structural similarity alone. This is especially true in those cases where a number of particulars are known regarding the characteristic domains of the receptor, etc. involved, to which the bioisosteres are bound or which works upon said bioisosteres in some manner.

Examples of bioisosteres for acid and base groups are shown below.

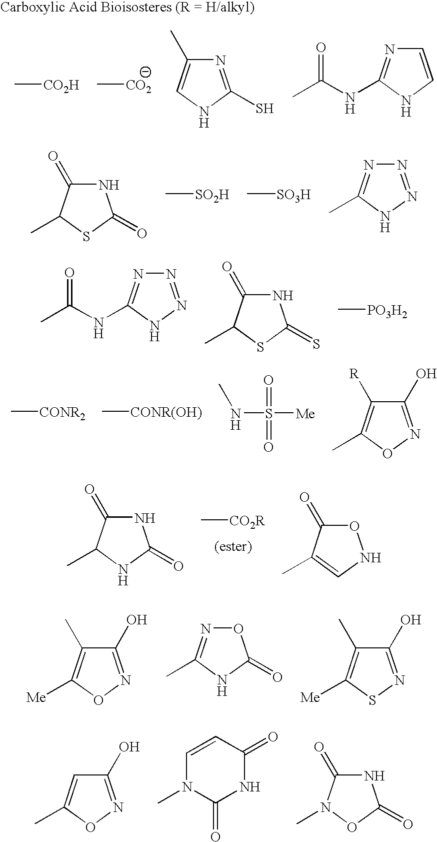

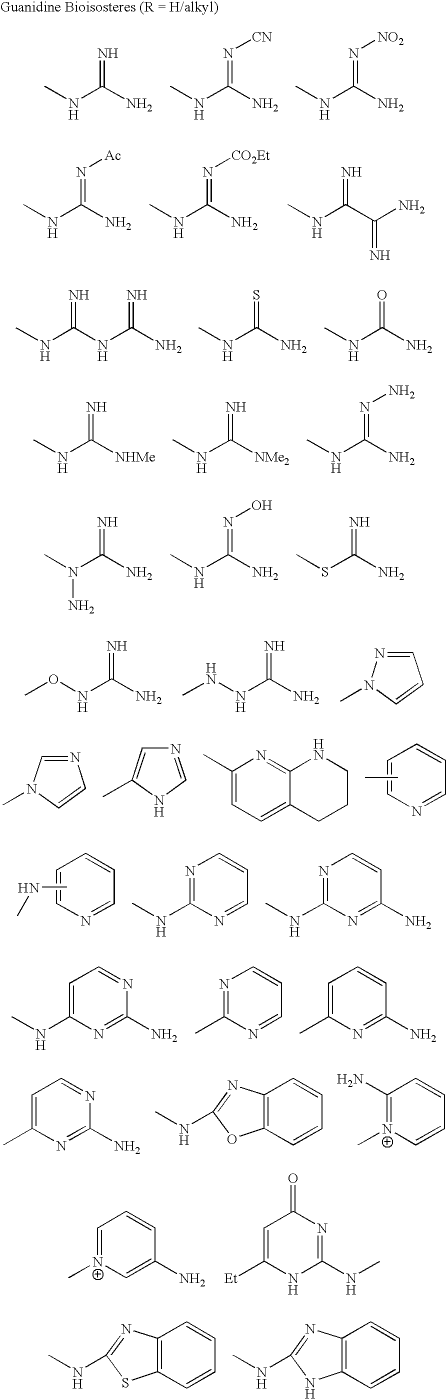

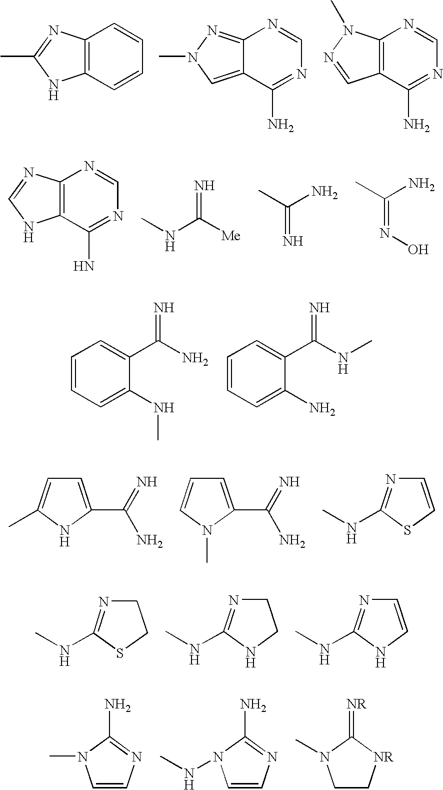

As used herein, the term “amino acid” can also refer to a molecule of the general formula NH₂—CHR—COOH or the residue within a peptide bearing the parent amino acid, where “R” is one of a number of different side chains. “R” can be a substituent referring to one of the twenty genetically coded amino acids. “R” can also be a substituent referring to one that is not of the twenty genetically coded amino acids. As used herein, the term “amino acid residue” refers to the portion of the amino acid which remains after losing a water molecule when it is joined to another amino acid. As used herein, the term “amino acid analog” refers to a structural derivative of an amino acid parent compound that differs from it by at least one element, such as for example, an alpha amino group or an acidic amino acid in which the acidic R group has been replaced with a bioisostere thereof. As such “half-denuded” and “denuded” embodiments of the present invention comprise amino acid analogs since these versions vary from a traditional amino acid structure in missing at least an element, such as an alpha amino or carboxy group. The term “modified amino acid” refers more particularly to an amino acid bearing an “R” substituent that does not correspond to one of the twenty genetically coded amino acids—as such modified amino acids fall within the broader class of amino acid analogs.

As used herein, the term “fully protected” refers to a preferred embodiment in which both the amino and carboxyl terminals comprise protecting groups.

As used herein, the term “half-denuded” refers to a preferred embodiment in which one of the alpha amino group or the alpha carboxy group is missing from the respective amino or carboxy terminal amino acid residues or analogs thereof. The remaining alpha amino or alpha carboxy group is capped with a protecting group.

As used herein, the term “denuded” or “fully-denuded” refers to a preferred embodiment in which both the alpha amino and alpha carboxy groups have been removed from the respective amino or carboxy terminal amino acid residues or analogs thereof.

Certain compounds can exist in tautomeric forms. All such isomers including diastereomers and enantiomers are covered by the embodiments. It is assumed that the certain compounds are present in either of the tautomeric forms or mixture thereof.

Certain compounds can exist in polymorphic forms. Polymorphism results from crystallization of a compound in at least two distinct forms. All such polymorphs are covered by the embodiments. It is assumed that the certain compounds are present in a certain polymorph or mixture thereof.

HMG-CoA Reductase Inhibition

As stated above, a scaffold is a mimic of a portion of an HMG CoA reductase inhibitor that is lipophilic or aromatic.

HMG CoA reductase inhibitors share a rigid, hydrophobic group which is linked to an HMG-like moiety. HMG CoA reductase inhibitors are competitive inhibitors of HMGR with respect to binding of the substrate HMG CoA. The structurally diverse, rigid hydrophobic groups of HMG CoA reductase inhibitors are accommodated in a shallow non-polar groove of HMGR.

Inhibition of HMGR is an effective and safe method in cholesterol lowering therapy. HMG CoA reductase inhibitors have other effects in addition to lowering cholesterol. These include nitric oxide mediated promotion of new blood vessel growth, stimulation of bone formation, protection against oxidative modification of low-density lipoprotein, anti-inflammatory effects, and reduction in C-reactive protein levels.

RCT Mediation

To date, efforts at designing ApoA-I agonists have focused on the 22-mer unit structures, e.g., the “consensus 22-mer” of Anantharamaiah et al., 1990, Arteriosclerosis 10(1):95-105; Venkatachalapathi et al., 1991, Mol. Conformation and Biol. Interactions, Indian Acad. Sci. B:585-596, which are capable of forming amphipathic α-helices in the presence of lipids. (See e.g., U.S. Pat. No. 6,376,464 directed at peptide mimetics derived from modifications of the consensus 22-mer). There are several advantages of using such relatively short peptides compared to longer 22-mers. For example, the shorter mediators of RCT are easier and less costly to produce, they are chemically and conformationally more stable, the preferred conformations remain relatively rigid, there is little or no intra-molecular interactions within the peptide chain, and the shorter peptides exhibit a higher degree of oral availability. Multiple copies of these shorter peptides might bind to the HDL or LDL producing the same effect of a more restrained large peptide. Although ApoA-I multifunctionality may be based on the contributions of its multiple α-helical domains, it is also possible that even a single function of ApoA-I, e.g., LCAT activation, can be mediated in a redundant manner by more than one of the α-helical domains. Thus, in a preferred aspect of the embodiments, multiple functions of ApoA-I may be mimicked by the disclosed mediators of RCT which are directed to a single sub-domain.

Three functional features of ApoA-I are widely accepted as major criteria for ApoA-I agonist design: (1) ability to associate with phospholipids; (2) ability to activate LCAT; and (3) ability to promote efflux of cholesterol from the cells. The molecular mediators of RCT in accordance with some modes of the preferred embodiments may exhibit only the last functional feature—ability to increase RCT. However, quite a few other properties of ApoA-I, which are often overlooked, make ApoA-I a particularly attractive target for therapeutic intervention. For example, ApoA-I directs the cholesterol flux into the liver via a receptor-mediated process and modulates pre-β-HDL (primary acceptor of cholesterol from peripheral tissues) production via a PLTP driven reaction. However, these features allow broadening of the potential usefulness of ApoA-I mimetic molecules. This, entirely novel approach to viewing ApoA-I mimetic function, will allow use of the peptides or amino acid-derived small molecules, which are disclosed herein, to facilitate direct RCT (via HDL pathway) as well as indirect RCT (i.e., to intercept and clear the LDLs from circulation, by redirecting their flux to the liver). To be capable of enhancing indirect RCT, the molecular mediators of the preferred embodiments will preferably be able to associate with phospholipids and bind to the liver (i.e., to serve as ligand for liver lipoprotein binding sites).

Thus, a goal of the research efforts which led to preferred embodiments was to identify, design, and synthesize the short stable small molecule mediators of RCT that exhibit preferential lipid binding conformation, increase cholesterol flux to the liver by facilitating direct and/or indirect reverse cholesterol transport, improve the plasma lipoprotein profile, and subsequently prevent the progression or/and promote the regression of atherosclerotic lesions.

The mediators of RCT of the preferred embodiments can be prepared in stable bulk or unit dosage forms, e.g., lyophilized products, that can be reconstituted before use in vivo or reformulated. Preferred embodiments of the invention includes the pharmaceutical formulations and the use of such preparations in the treatment of hyperlipidemia, hypercholesterolemia, coronary heart disease, atherosclerosis, diabetes, obesity, Alzheimer's Disease, multiple sclerosis, conditions related to hyperlipidemia, such as inflammation, and other conditions such as endotoxemia causing septic shock.

The preferred embodiments are illustrated by working examples which demonstrate that the mediators of RCT of the preferred embodiments associate with the HDL and LDL component of plasma, and can increase the concentration of HDL and pre-β-HDLparticles, and lower plasma levels of LDL. Thus promote direct and indirect RCT. The mediators of RCT increase human LDL mediated cholesterol accumulation in human hepatocytes (HepG2 cells). The mediators of RCT are also efficient at activating PLTP and thus promote the formation of pre-β-HDL particles. Increase of HDL cholesterol served as indirect evidence of LCAT involvement (LCAT activation was not shown directly (in vitro)) in the RCT. Use of the mediators of RCT of the preferred embodiments in vivo in animal models results in an increase in serum HDL concentration.

The preferred embodiments are set forth in more detail in the subsections below, which describe composition and structure of the mediators of RCT, including lipophilic scaffolds derived from HMG CoA reductase inhibitors, including protected versions, half denuded versions, and denuded versions thereof; structural and functional characterization; methods of preparation of bulk and unit dosage formulations; and methods of use.

Mediator Structure and Function

In some preferred embodiments, the mediators of RCT are generally peptides, or analogues thereof, which mimic the activity of ApoA-I. In some embodiments, at least one amide linkage in the peptide is replaced with a substituted amide, an isostere of an amide or an amide mimetic. Additionally, one or more amide linkages can be replaced with peptidomimetic or amide mimetic moieties which do not significantly interfere with the structure or activity of the peptides. Suitable amide mimetic moieties are described, for example, in Olson et al., 1993, J. Med. Chem. 36:3039-3049.

As used herein, the abbreviations for the genetically encoded L-enantiomeric amino acids are conventional and are as follows: The D-amino acids are designated by lower case, e.g. D-alanine=a, etc.

TABLE 1

| Amino Acids | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Certain amino acid residues in the mediators of RCT can be replaced with other amino acid residues without significantly deleteriously affecting, and in many cases even enhancing, the activity of the peptides. Thus, also contemplated by the preferred embodiments are altered or mutated forms of the mediators of RCT wherein at least one defined amino acid residue in the structure is substituted with another amino acid residue or derivative and/or analog thereof. It will be recognized that in preferred embodiments, the amino acid substitutions are conservative, i.e., the replacing amino acid residue has physical and chemical properties that are similar to the amino acid residue being replaced.

For purposes of determining conservative amino acid substitutions, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into nonpolar and aromatic amino acids. The definitions of the various categories of amino acids that define ApoA-I are as follows:

The term “hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gln (Q), Asp (D), Lys (K) and Arg (R).

The term “hydrophobic amino acid” refers to an amino acid exhibiting a hydrophobicity of greater than zero according to the normalized consensus hydrophobicity scale of Eisenberg, 1984, J. Mol. Biol. 179:1.25-142. Genetically encoded hydrophobic amino acids include Pro (P), Ile (I), Phe (F), Val (V), Leu (L), Trp (W), Met (M), Ala (A), Gly (G) and Tyr (Y).

The term “acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

The term “basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The amino acid residue Cys (C) is unusual in that it can form disulfide bridges with other Cys (C) residues or other sulfanyl-containing amino acids. The ability of Cys (C) residues (and other amino acids with —SH containing side chains) to exist in a peptide in either the reduced free —SH or oxidized disulfide-bridged form affects whether Cys (C) residues contribute net hydrophobic or hydrophilic character to a peptide. While Cys (C) exhibits a hydrophobicity of 0.29 according to the normalized consensus scale of Eisenberg (Eisenberg, 1984, supra), it is to be understood that for purposes of the preferred embodiments Cys (C) is categorized as a polar hydrophilic amino acid, notwithstanding the general classifications defined above.

As will be appreciated by those of skill in the art, the above-defined categories are not mutually exclusive. Thus, amino acids having side chains exhibiting two or more physical-chemical properties can be included in multiple categories. For example, amino acid side chains having aromatic moieties that are further substituted with polar substituents, such as Tyr (Y), may exhibit both aromatic hydrophobic properties and polar or hydrophilic properties, and can therefore be included in both the aromatic and polar categories. The appropriate categorization of any amino acid will be apparent to those of skill in the art, especially in light of the detailed disclosure provided herein.

While the above-defined categories have been exemplified in terms of the genetically encoded amino acids, the amino acid substitutions need not be, and in certain embodiments preferably are not, restricted to the genetically encoded amino acids. Indeed, many of the preferred mediators of RCT contain genetically non-encoded amino acids. Thus, in addition to the naturally occurring genetically encoded amino acids, amino acid residues in the mediators of RCT may be substituted with naturally occurring non-encoded amino acids and synthetic amino acids.

Certain commonly encountered amino acids which provide useful substitutions for the mediators of RCT include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); omithine (Om); citrulline (Cit); t-butylalanine (t-BuA); t-butylglycine (t-BuG); N-methylisoleucine (MeIle); phenylglycine (Phg); cyclohexylalanine (Cha); norleucine (Nle); naphthylalanine (Nal); 4-phenylphenylalanine, 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe (pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

Other amino acid residues not specifically mentioned herein can be readily categorized based on their observed physical and chemical properties in light of the definitions provided herein.

The classifications of the genetically encoded and common non-encoded amino acids according to the categories defined above are summarized in Table 2, below. It is to be understood that Table 2 is for illustrative purposes only and does not purport to be an exhaustive list of amino acid residues and derivatives that can be used to substitute the mediators of RCT described herein.

TABLE 2
CLASSIFICATIONS OF COMMONLY
ENCOUNTERED AMINO ACIDS

| Classification | Genetically Encoded | Non-Genetically Encoded |
|---|---|---|
| Hydrophobic | | |
| Aromatic | F, Y, W | Phg, Nal, Thi, Tic, |
| | | Phe (4-Cl), Phe (2-F), |
| | | Phe (3-F), Phe (4-F), hPhe |
| Nonpolar | L, V, I, M, G, A, P | t-BuA, t-BuG, MeIle, Nle, |
| | | MeVal, Cha, McGly, Aib |
| Aliphatic | A, V, L, I | b-Ala, Dpr, Aib, Aha, |
| | | MeGly, t-BuA, t-BuG, |
| | | MeIle, Cha, Nle, MeVal |
| Hydrophilic | | |
| Acidic | D, E | |
| Basic | H, K, R | Dpr, Orn, hArg, |
| | | Phe (p-NH₂), Dbu, Dab |
| Polar | C, Q, N, S. T | Cit, AcLys, MSO, bAla, hSer |
| Helix-Breaking | P, G | D-Pro and other D-amino |
| | | acids (in L-peptides) |

Other amino acid residues not specifically mentioned herein can be readily categorized based on their observed physical and chemical properties in light of the definitions provided herein.

While in most instances, the amino acids of the mediators of RCT will be substituted with D-enantiomeric amino acids, the substitutions are not limited to D-enantiomeric amino acids. Thus, also included in the definition of “mutated” or “altered” forms are those situations where an D-amino acid is replaced with an identical L-amino acid (e.g., D-Arg-L-Arg) or with a L-amino acid of the same category or subcategory (e.g., D-Arg D-Lys), and vice versa. The mediators may advantageously be composed of at least one D-enantiomeric amino acid. Mediators containing such D-amino acids are thought to be more stable to degradation in the oral cavity, gut or serum than are molecules composed exclusively of L-amino acids.

Linkers

The mediators of RCT can be connected or linked in a head-to-tail fashion (i.e., N-terminus to C-terminus), a head-to-head fashion, (i.e., N-terminus to N-terminus), a tail-to-tail fashion (i.e., C-terminus to C-terminus), or combinations thereof. The linker can be any bifunctional molecule capable of covalently linking two peptides to one another. Thus, suitable linkers are bifunctional molecules in which the functional groups are capable of being covalently attached to the N- and/or C-terminus of a peptide. Functional groups suitable for attachment to the N- or C-terminus of peptides are well known in the art, as are suitable chemistries for effecting such covalent bond formation.

Linkers of sufficient length and flexibility include, but are not limited to, Pro (P), Gly (G), Cys-Cys,Gly-Gly, H₂N—CH₂)ₙ—COOH where n is 1 to 12, preferably 4 to 6; H₂N-aryl-COOH and carbohydrates. However, in some embodiments, no separate linkers per se are used at all. Instead, the acidic, lipophilic and basic moitites are all part of a single molecule.

HMG CoA Reductase Inhibitors Scaffold

In preferred embodiments, the hydrophobic or aromatic scaffold is based on an HMG CoA reductase inhibitor. Examples of HMG CoA reductase inhibitors are shown below:

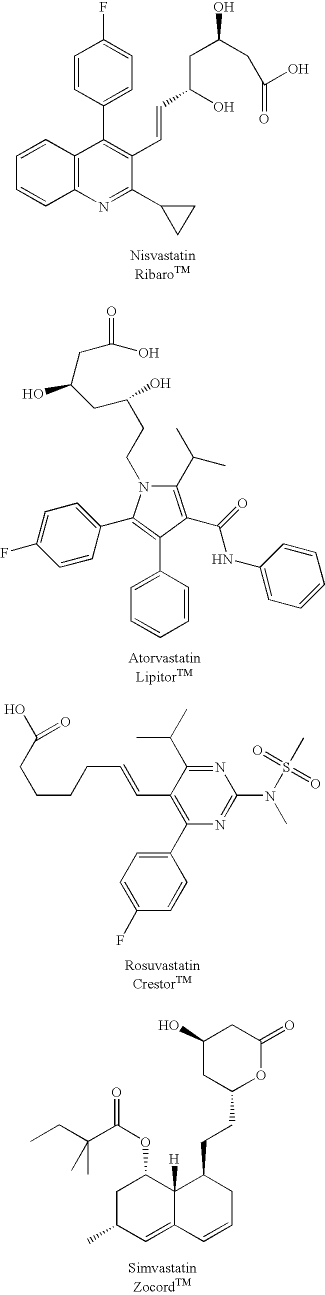

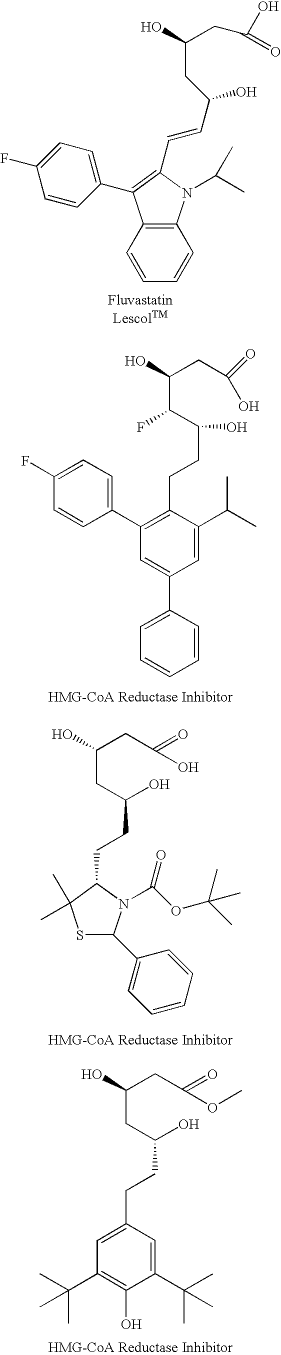

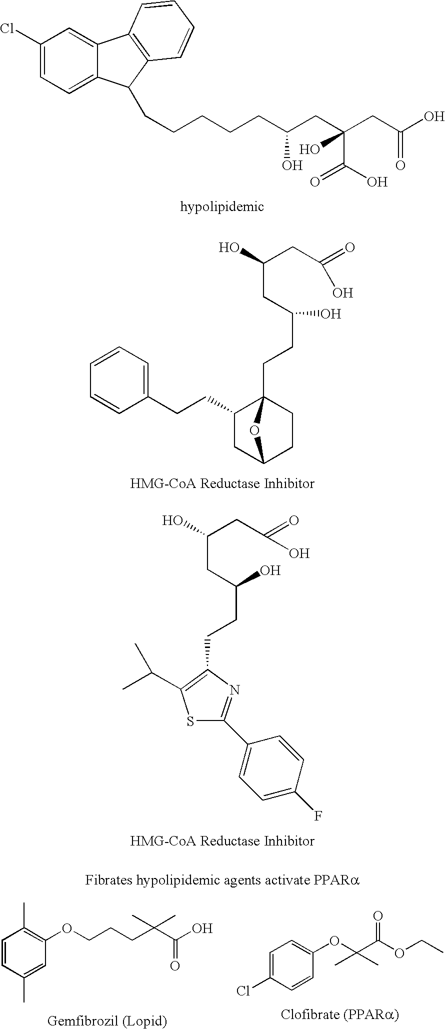

Accordingly, examples of lipophilic or aromatic scaffolds based on HMG CoA reductase inhibitors are shown below along with the parent HMG CoA reductase inhibitor:

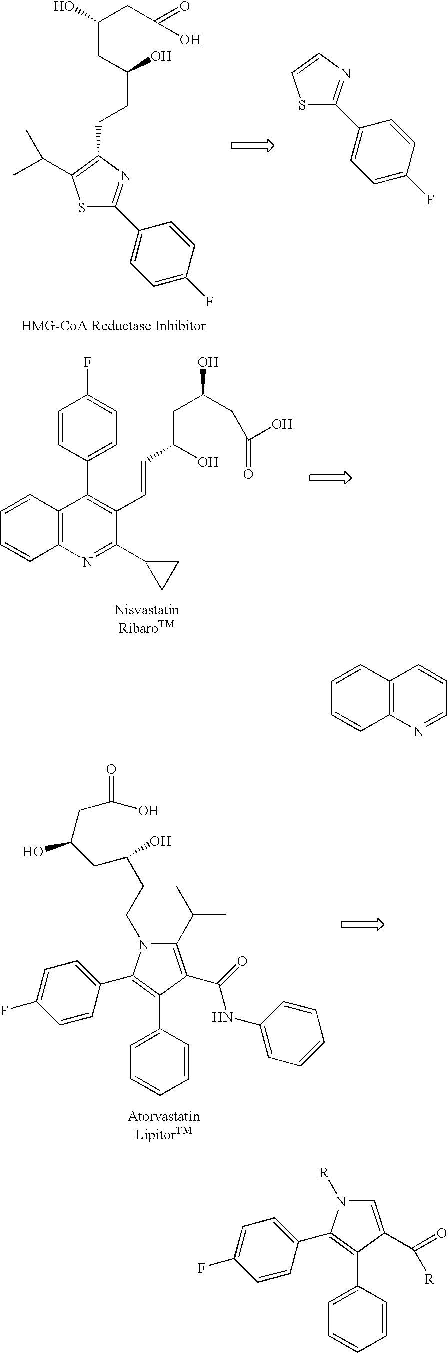

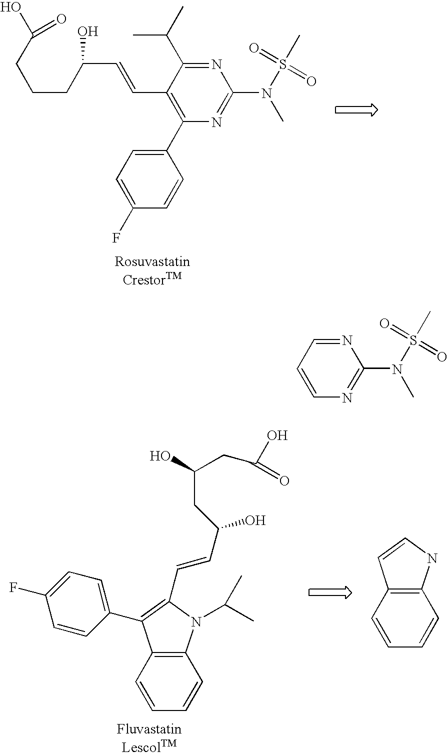

Examples of RCT mediators that comprise a lipophilic scaffold based on an HMG CoA reductase inhibitor, such as nisvastatin, are shown below.

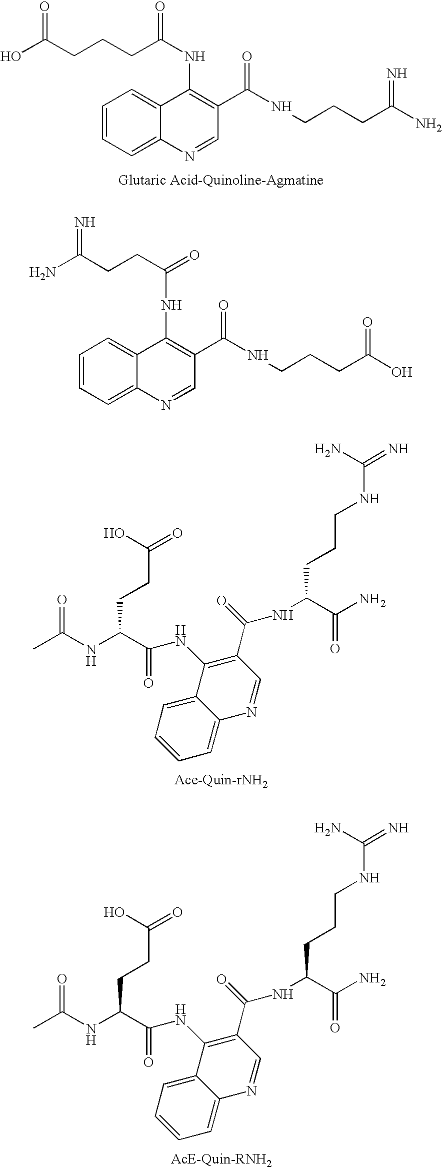

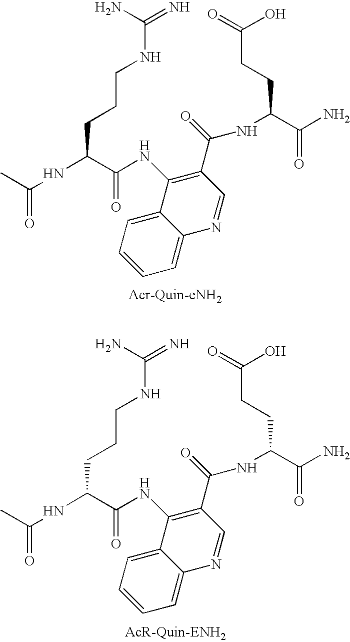

As stated above, preferably, a scaffold is a mimic of a portion of an HMG CoA reductase inhibitor that is lipophilic or aromatic. HMG CoA reductase inhibitors share a rigid, hydrophobic group which is linked to an HMG-like moiety. HMG CoA reductase inhibitors are competitive inhibitors of HMGR with respect to binding of the substrate HMG CoA. The structurally diverse, rigid hydrophobic groups of HMG CoA reductase inhibitors are accommodated in a shallow non-polar groove of HMGR.

HMG CoA reductase inhibitor scaffold substituted alanine derivatives are replacements of the central amino acid (X₂) in the X1-X2-X3, X1-X2-Y3, Y1-X2-X3 or Y1-X2-Y3 molecular models; although the molecules can be rearranged in any order. The amino acid derivatives can be prepared from the corresponding aryl aldehydes (J—CHO, where J is any of the stain scaffolds), as shown below. The amino acid derivatives can be prepared in enantiomerically pure (D or L, depending on the chiral catalyst) or in the racemic form.

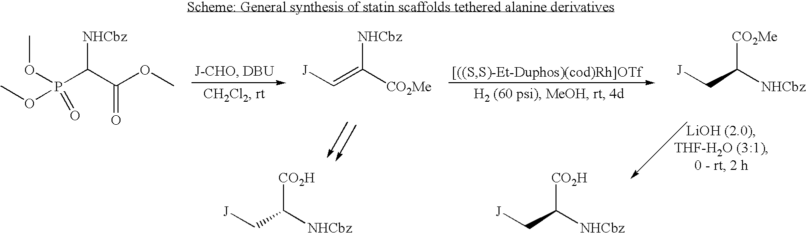

The above-mentioned aryl aldehydes (Jₙ—CHO, n=1-4) can be prepared according to the following schemes.

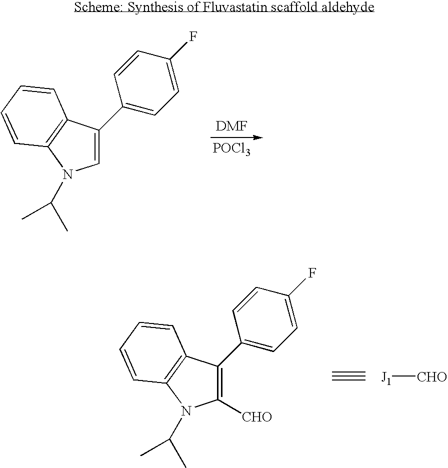

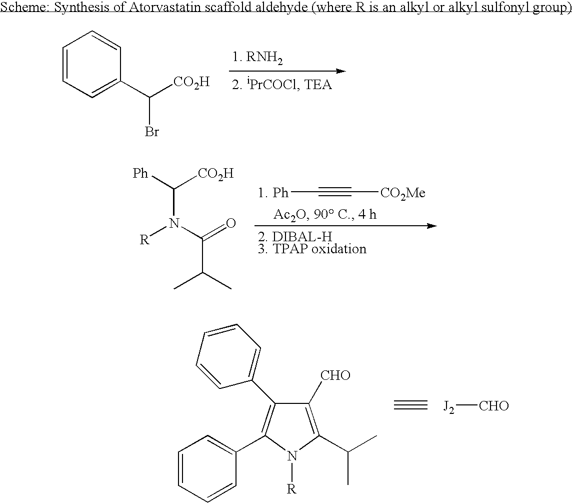

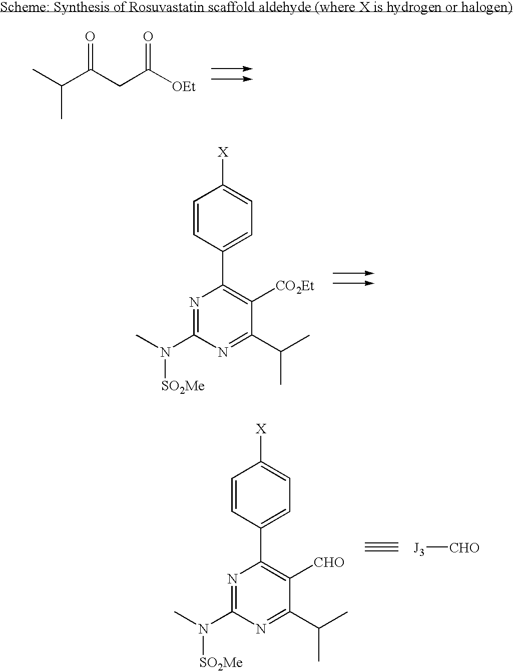

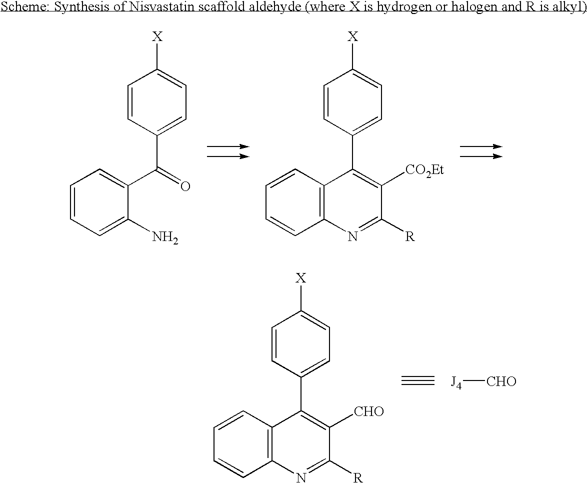

These statin substituted alanine derivatives can then be coupled with other amino acid derivatives (e.g., Glu or Arg). Further, these derivatives can be denuded partly or fully, as described in the case of EFR or efr.

One embodiment of the RCT mediators using an HMG CoA reductase scaffold is based on atorvastatin.

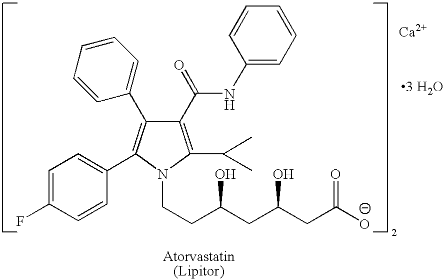

The D- and L-amino acid derivatives based on atorvastatin can be synthesized. These derivatives further can be denuded partly or fully. The bioisosteric replacement can be done at one of the amino acid residues or both together. The glutamic acid moiety can be replaced, for example, by 3-amino benzoic acid or PABA. These derivatives are shown in the following charts and schemes.

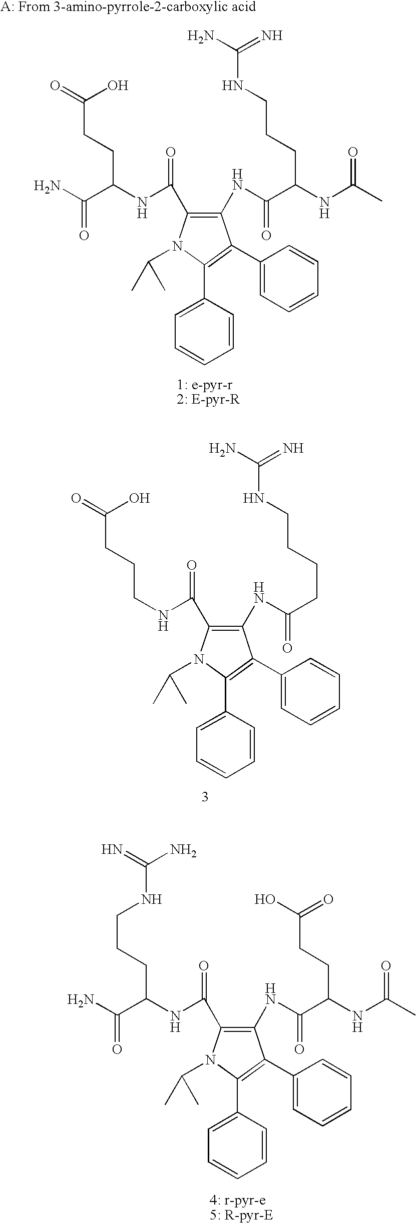

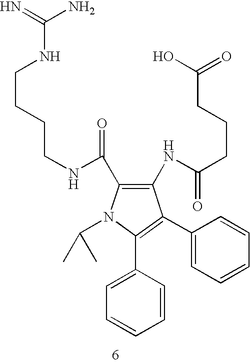

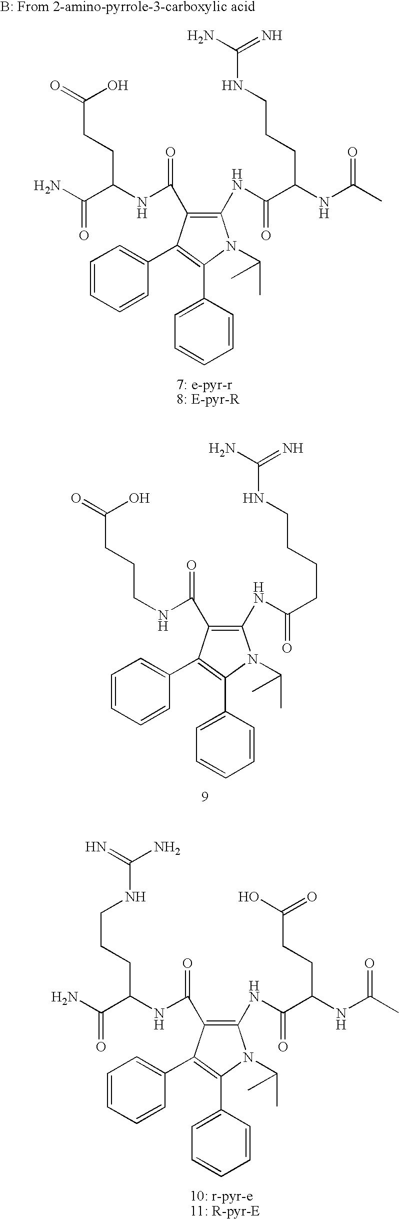

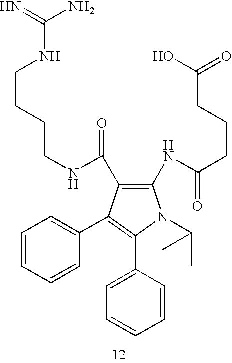

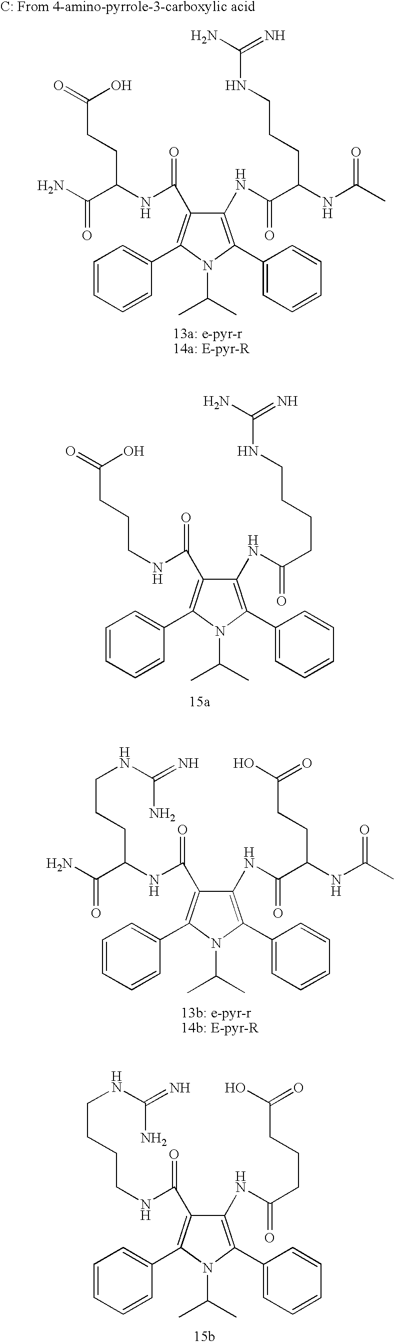

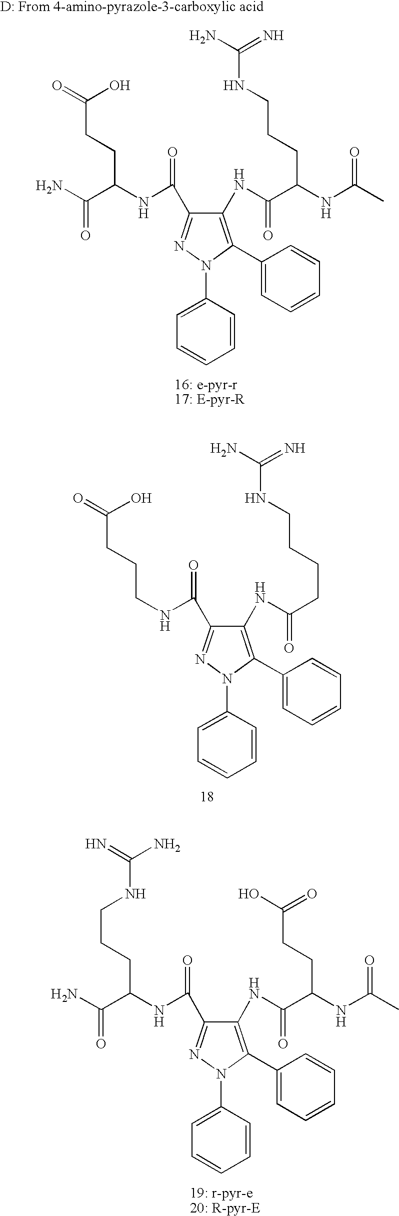

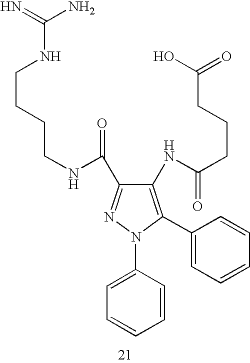

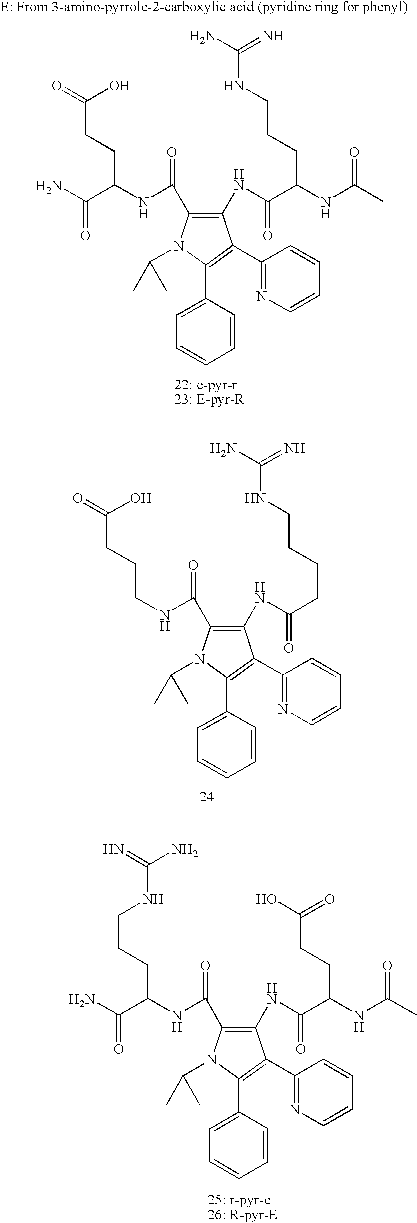

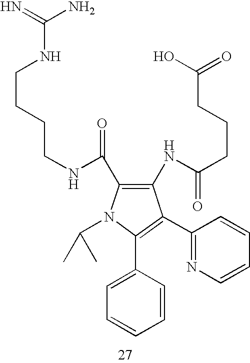

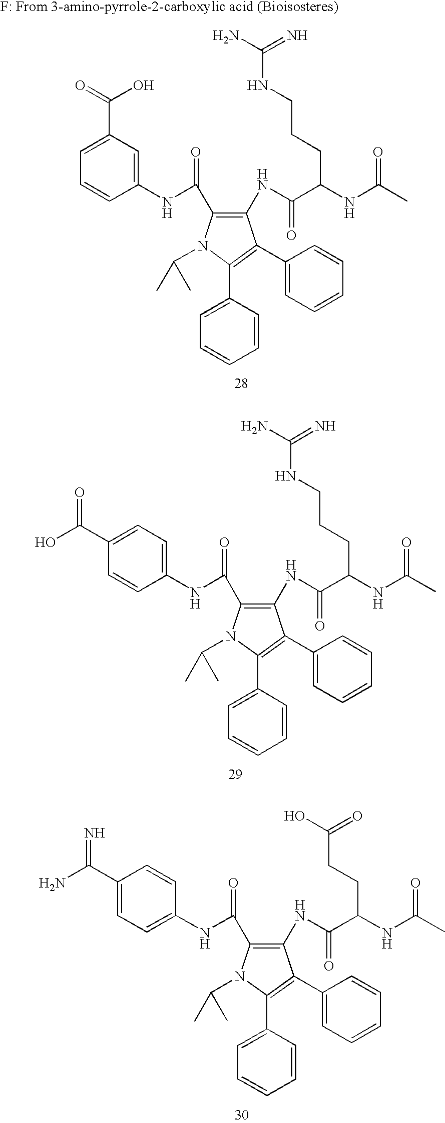

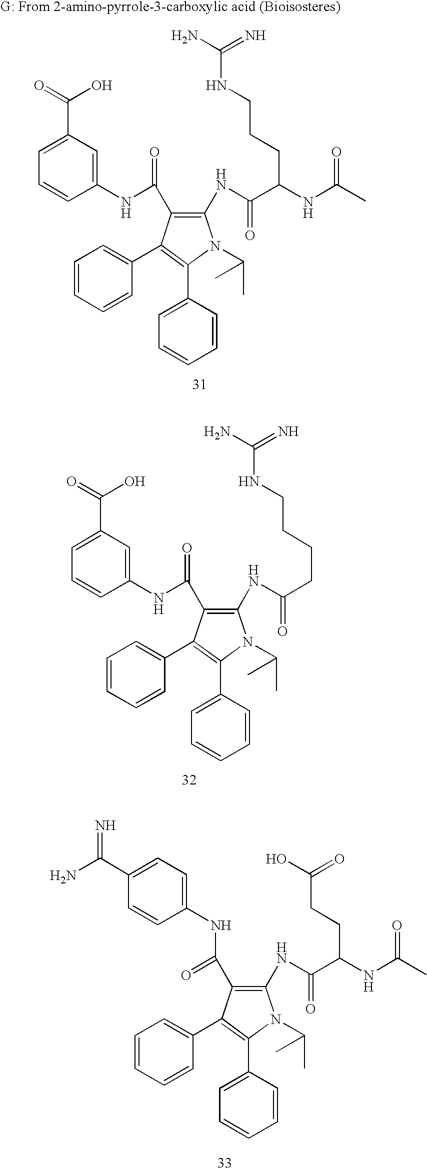

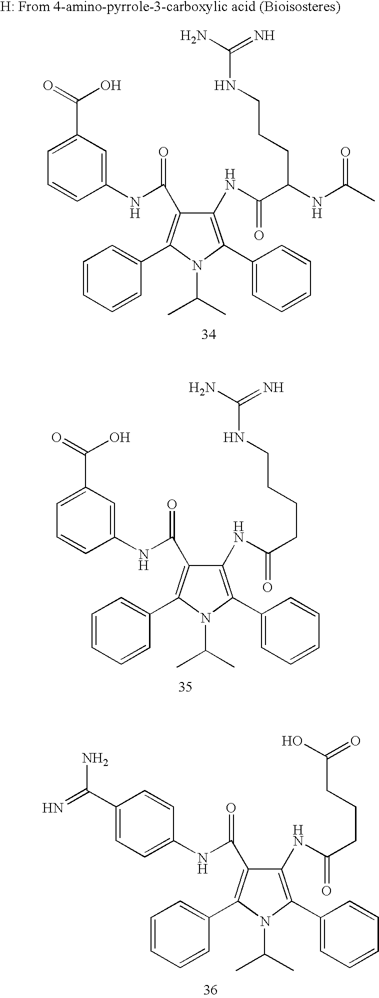

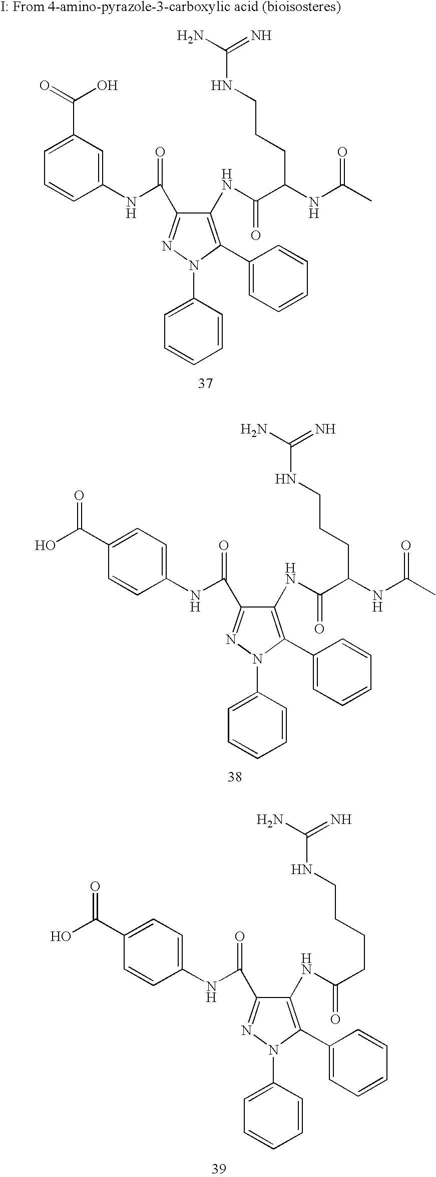

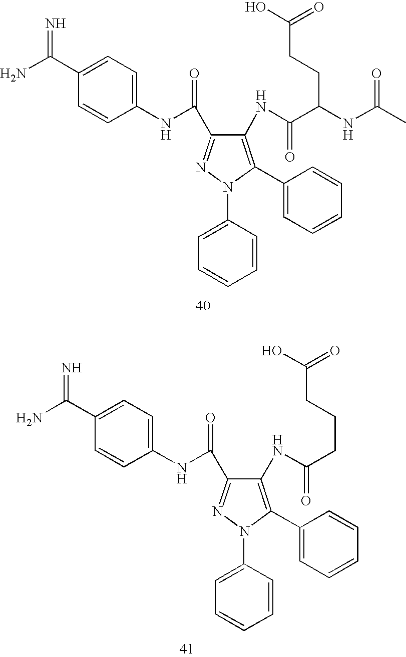

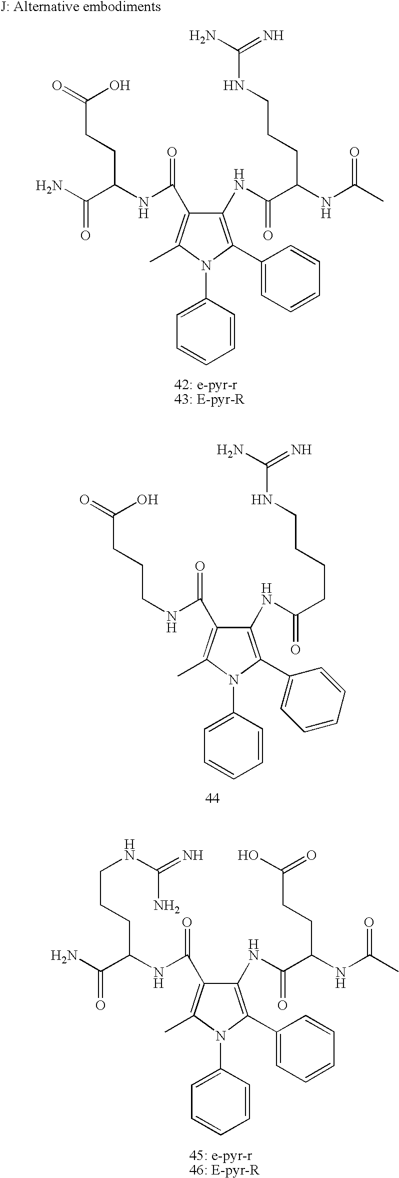

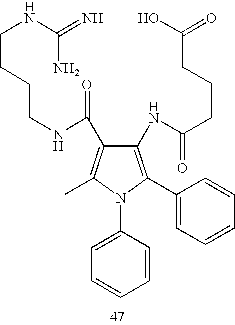

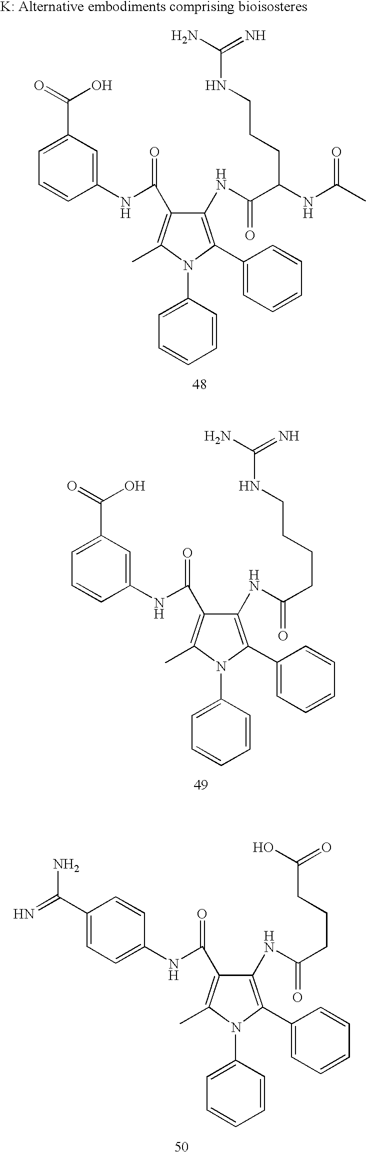

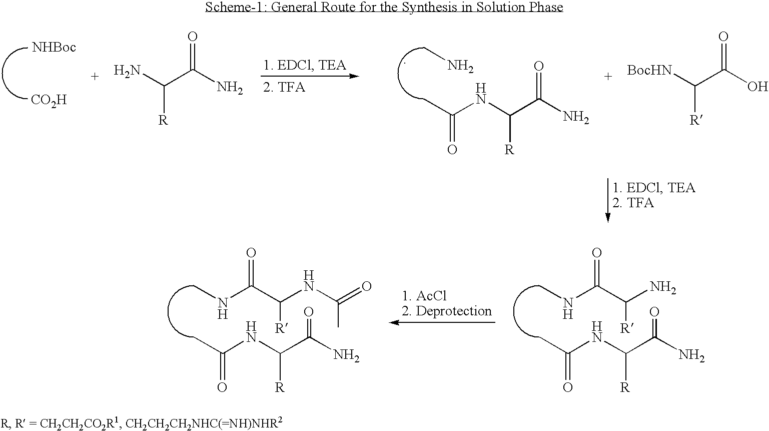

The general route for the N-Boc protected amino acids in solution phase peptide synthesis is shown in Scheme 1. First, the acid is reacted with the amine under standard conditions (e.g., EDCI, HOBt, Et₃N) and the resulting product is deprotected (TFA) to the corresponding amine. The latter is coupled with another appropriately protected amino acid under the standard conditions. The N-Boc is removed (TFA) and capped with acid chloride (e.g. AcCl) and the other protecting groups are removed to the desired product.

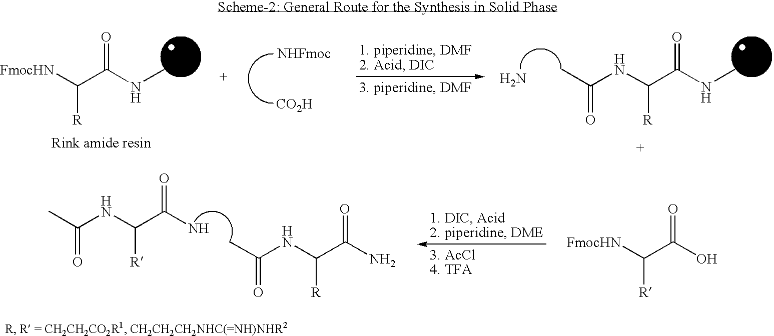

The general route for the N-Boc protected amino acids in solid phase peptide synthesis is shown in Scheme 2. First, the N-Fmoc of the resin (Rink) is deprotected (piperidine, DMF) and then coupled with an N-Fmoc protected amino acid under standard conditions (e.g., DIC, HOBt, Et₃N) and the resulting product is deprotected as above to the resin-bound amido-amine. The latter is coupled with another appropriately protected amino acid under the standard conditions and repeated once more. The N-Fmoc is removed (piperidine, DMF) and capped with acid chloride (e.g. AcCl) and the other protecting groups are removed to the desired product.
The Scaffold Intermediates:

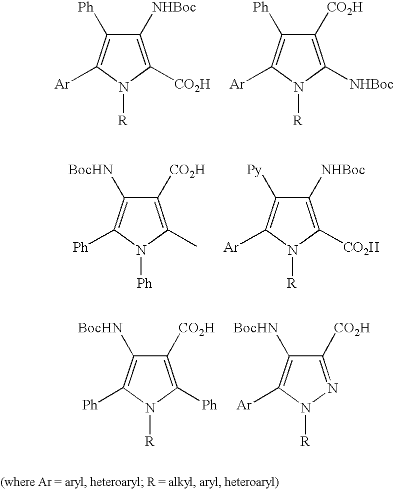

The scaffold replacements are shown above. Though, the N-Fmoc & N-Cbz derivatives are not shown, prepared as well. The syntheses of the latter intermediates are not depicted in the schemes but prepared in a similar fashion (using FmocCl) as their N-Boc derivatives. The schemes below describe the synthesis of these valuable intermediates.

The synthesis of 2-amino-pyrrole-3-carboxylic acid derivatives is shown in Scheme 3. Benzoin is reacted with SOCl₂ to the corresponding chloride, and then reacted with amine the alpha-keto amine. Alternatively, the latter is prepared directly from benzoin, when heated the amine in presence of a weaker acid (e. g. AcOH) in alcohol solvent. The amine is not monomer, instead, is oligomeric in nature (from Mass and proton NMR). The alpha-keto amine is reacted with dimethyl acetylenedicarboxylate (DMAD) in MeOH to the pyrrole product in good yield. The ester at 2-position is selectively hydrolyzed with 1 equivalent of aqueous NaOH in MeOH and acidified with dilute HCl. The resulting acid is submitted under Curtius rearrangement [diphenyl prosphoryl azide (DPPA), tert-BuOH, heat). The N-Boc protected ester is hydrolyzed (aq. NaOH, heat, then dilute HCl) to the corresponding acid.

Alternatively, the alpha-keto amine is reacted with ethyl cyanoacetate to the 2-amino-pyrrole (Scheme-3) and the latter is hydrolyzed and N-Boc protected under standard conditions.

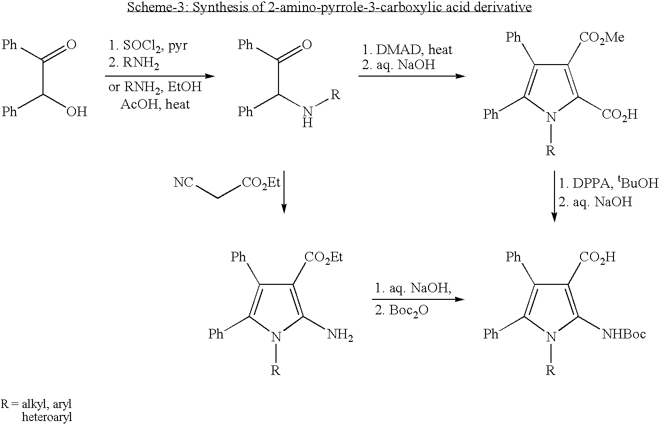

The syntheses of 3-amino-pyrrole-2-carboxylic acid derivatives are shown in Scheme 4 and Scheme 5. The amine is reacted with alpha-bromo-phenyl acetic acid, followed by treatment with acid chloride. The resulting amido-acid is treated with a dipolarophile (aryl-acetylene) in acetic anhydride to the pyrrole. The latter is successively nitrated (HNO₃ or nitronium salt), reduced (Raney-Nickel, H₂, EtOH/THF), hydrolyzed (aq. NaOH, heat) and N-protected (Boc₂O, dioxane) to the desired product (Scheme 4).

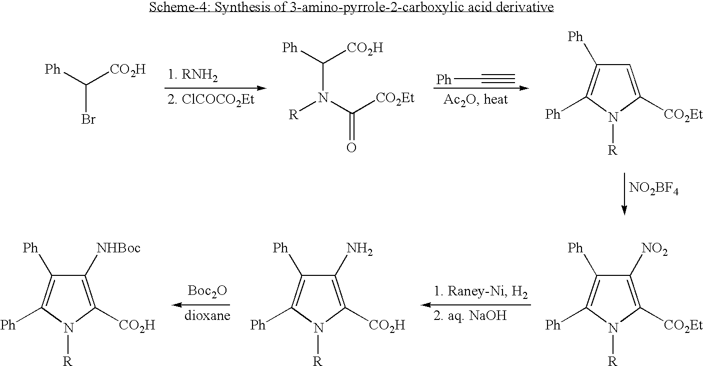

Scheme 5 shows the synthesis of heteroaryl-tethered pyrrole nucleus. The amido-acid is prepared similarly as shown previously (Scheme 4). The pyrrole nucleus is nitrated (HNO3 of nitronium salt). The latter is then reduced, hydrolyzed, N-Boc protected as given in Scheme 5.

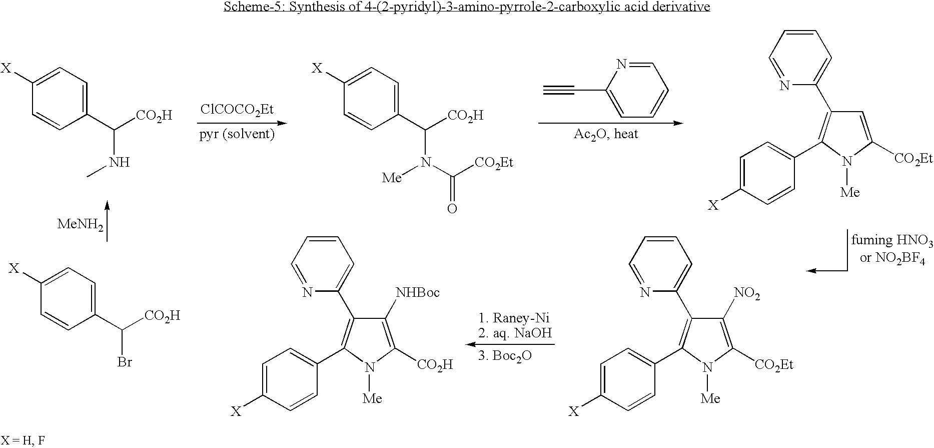

For the synthesis of the 4-amino-pyrrole-3-carboxylic acid derivatives, two routes are presented, as shown in Scheme 5 and Scheme 6. The alpha-amino acid is reacted with an acid chloride in pyridine to the N-acyl compound, which is heated with dimethyl acetylenedicarboxylate (DMAD) in acetic anhydride to the expected symmetrical pyrrole. The diacid is selectively hydrolyzed (1.0 equivalent aq. NaOH; dilute HCl) to the monoacid. The latter is treated with diphenyl phosprorylazide (DPPA) [benzene, tert-BuOH, heat] and aq. NaOH [heat; dilute HCl) to the desired compound (Scheme 6).

Alternatively, the amido-acid is reacted with propargyl ester in acetic anhydride, followed by nitration to the nitro-acid (Scheme 6). The nitro group then is reduced (Raney-Nickel, H₂, EtOH/THF) to the amine, the ester is hydrolyzed (Aq. NaOH) and the anime is protected (Boc₂O, dioxane) according to the Scheme 4.

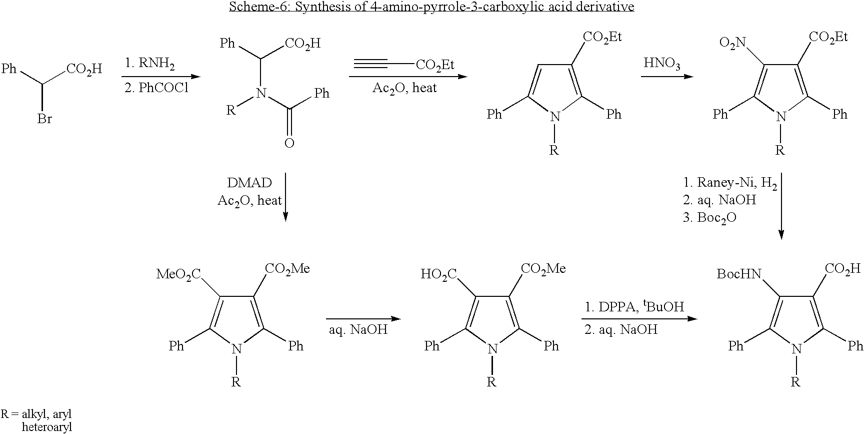

A completely different approach for the synthesis of the 4-amino-pyrrole-3-carboxylic acid derivatives is sketched in Scheme 7. First, a beta-keto ester is alkylated at the alpha position and the resulting diketo ester is treated with an amine to the pyrrole-3-carboxylate. The latter is converted to the desired product as shown in Scheme 6.

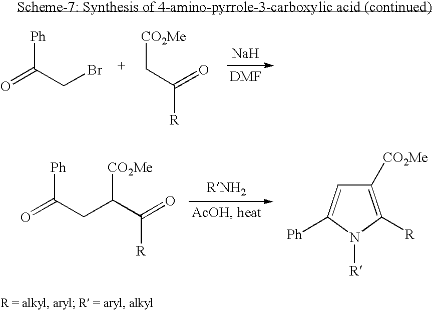

The synthesis of pyrazole nucleus is outlined in Scheme 8. In presence of base, aryl ketone is reacted with oxalate ester, followed by acidification to the 1,3-diketo-compound. The latter is reacted with a substituted hydrazine to pyrazole-3-carboxylate derivative. Subsequent nitration (HNO3 or nitronium salt), reduction (Raney-Nickel, H₂), ester hydrolysis (aq. NaOH), and amine protection (Boc₂O) lead tom the desired compound.

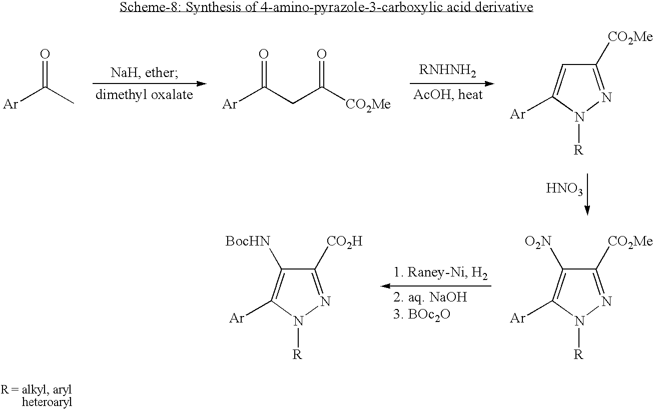

Another embodiment of the RCT mediators using an HMG CoA reductase scaffold is based on nisvastatin, as shown below. A general scheme to the synthesis of these compounds is also shown. 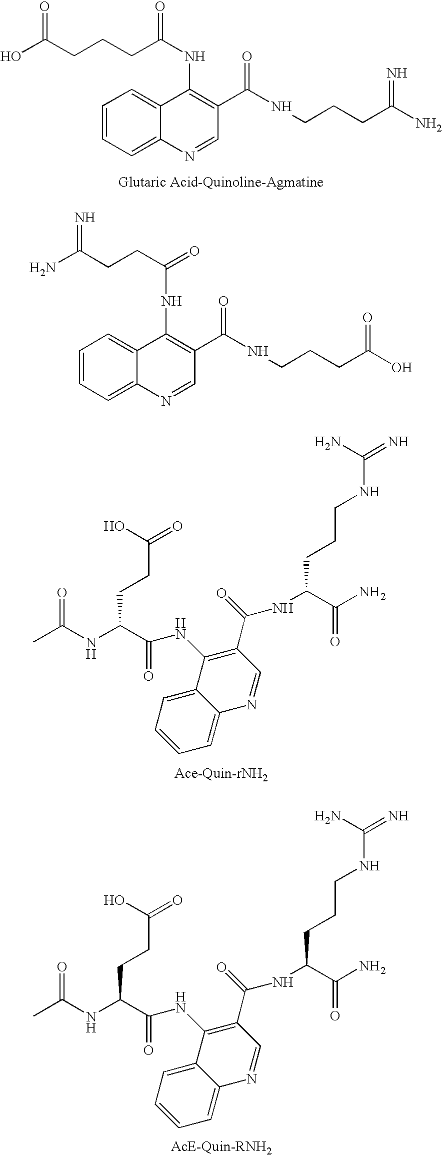 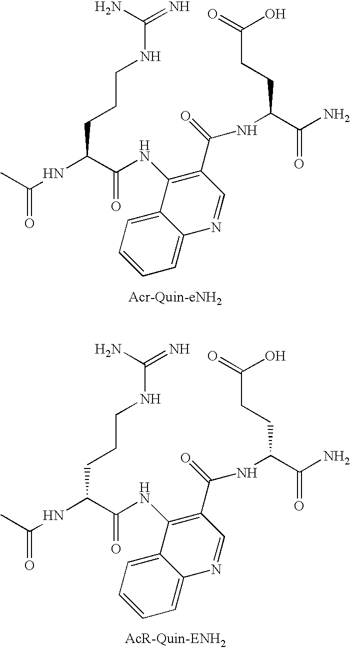
Bioisosteres Used Within the Structures of the Mediators of RCT

Examples of preferred bioisosteres that can be used within preferred RCT mediators are shown below. Bioisosteres containing a guanidium or amidino group serve to substitute an amino acid, such as arginine. Bioisosteres containing a carboxylic acid serve to substitute an amino acid, such as glutamate. Any other bioisostere that can serve to substitute the basic amino acids, arginine, lysine, or histidine, and the acidic amino acids, glutamate and aspartate are contemplated. Circles represent cyclic structures, including non-aromatic and aromatic structures.

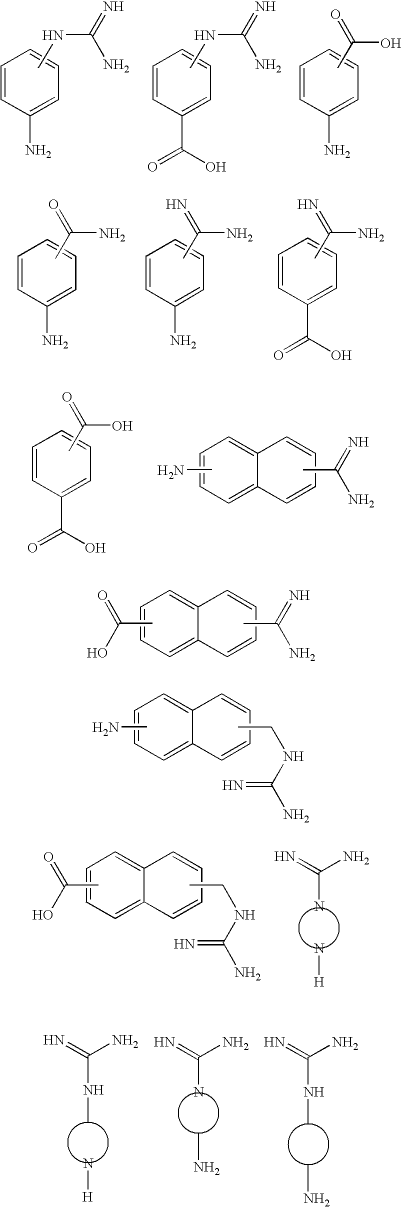

The synthetic schemes below show examples of methods that can be used to synthesize RCT mediators bearing bioisosteres. The term “AA” can represent a lipophilic scaffold in the schemes.

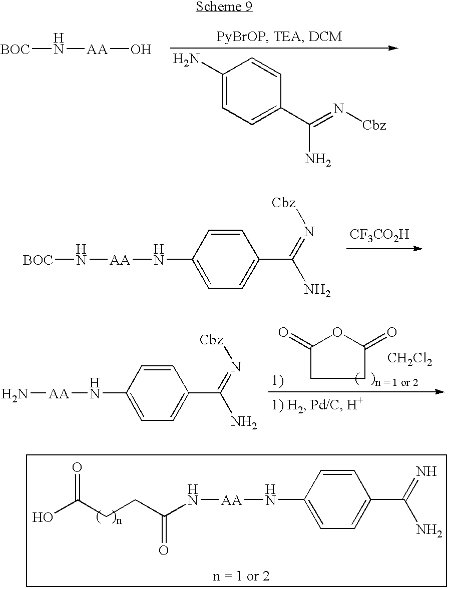

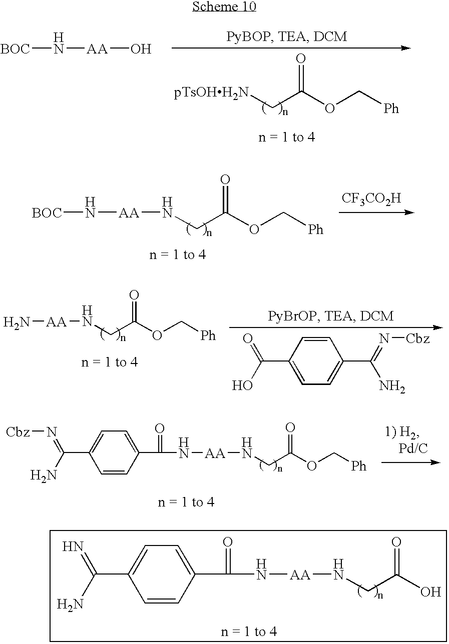

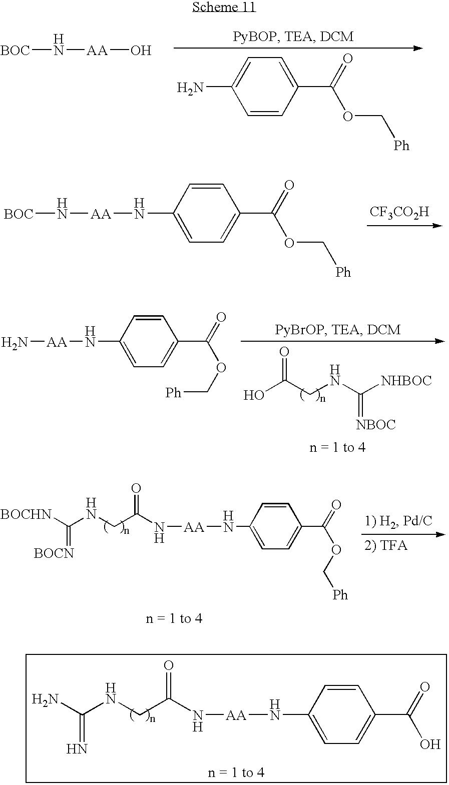

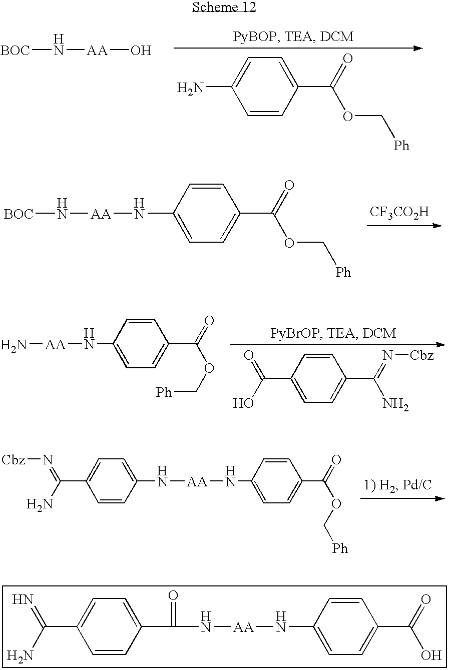

Examples of bioisosteres for carboxylic acid and guanidine groups are shown below. 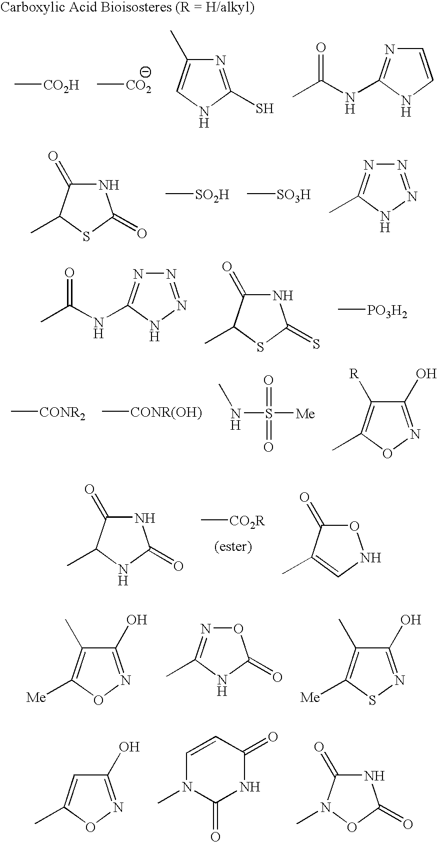 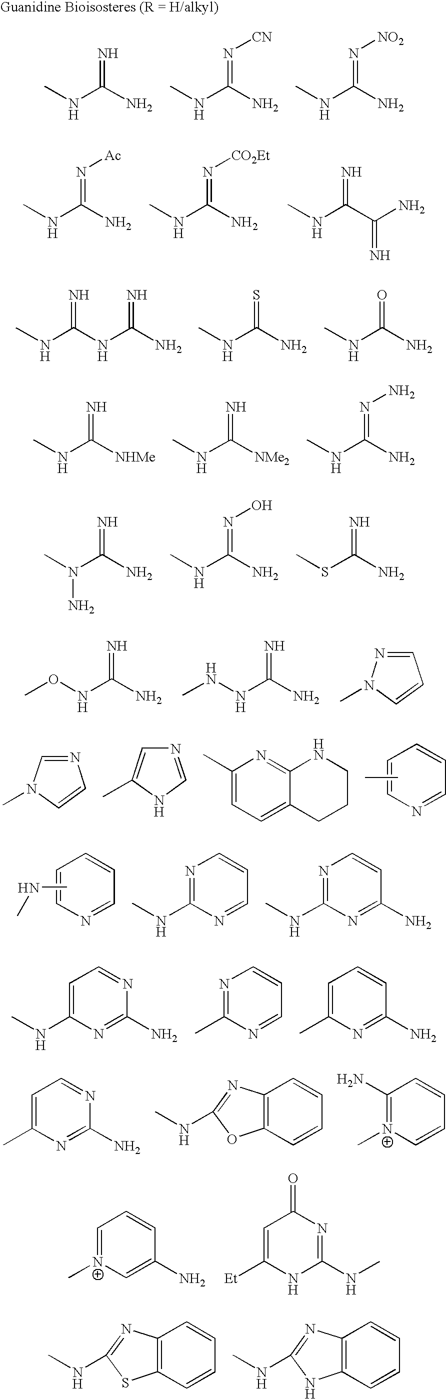 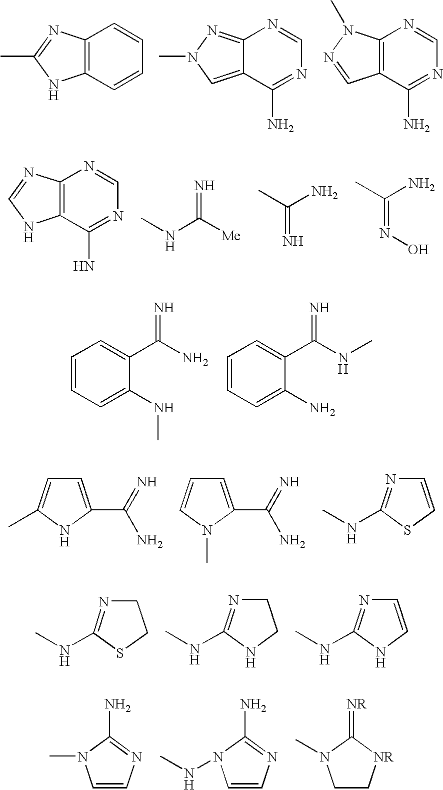
Preferred Mediators

In preferred embodiments, the mediator may be selected from the group consisting of 4-Agmatine-3-amidoGABAquinoline, 4-(1-(4-aminobutylcarbamoyl)-2-(2-methyl-4-phenylquinolin-3-yl)ethylcarbamoyl)butanoic acid, and 4-(5-guanidinopentylamino)quinoline-3-carboxylic acid. Any underivatized amino and/or carboxy terminal amino acid residues in the above list of preferred compounds are capped with a protecting group. In another preferred embodiment, the mediator has the structure: 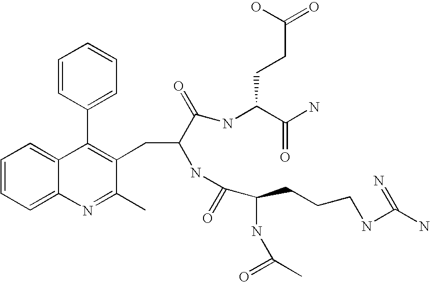
Analysis of Structure and Function

The structure and function of the mediators of RCT of the preferred embodiments, including the multimeric forms described above, can be assayed in order to select active compounds. For example, the mediators can be assayed for their ability to form α-helices, to bind lipids, to form complexes with lipids, to activate LCAT, and to promote cholesterol efflux, etc.

Methods and assays for analyzing the structure and/or function of the mediators are well-known in the art. Preferred methods are provided in the working examples, infra. For example, the circular dichroism (CD) and nuclear magnetic resonance (NMR) assays described, infra, can be used to analyze the structure of the mediators—particularly the degree of helicity in the presence of lipids. The ability to bind lipids can be determined using the fluorescence spectroscopy assay described, infra. The ability of the mediators to activate LCAT can be readily determined using the LCAT activation described, infra. The in vitro and in vivo assays described, infra, can be used to evaluate the half-life, distribution, cholesterol efflux and effects on RCT.

Synthetic Methods

The preferred embodiments may be prepared using virtually any art-known technique for the preparation of compounds. For example, the compounds may be prepared using conventional step-wise solution or solid phase peptide syntheses.

The mediators of RCT may be prepared using conventional step-wise solution or solid phase synthesis (see, e.g., Chemical Approaches to the Synthesis of Peptides and Proteins, Williams et al., Eds., 1997, CRC Press, Boca Raton Fla., and references cited therein; Solid Phase Peptide Synthesis: A Practical Approach, Atherton & Sheppard, Eds., 1989, IRL Press, Oxford, England, and references cited therein).

In conventional solid-phase synthesis, attachment of the first amino acid or analog thereof entails chemically reacting its carboxyl-terminal (C-terminal) end with derivatized resin to form the carboxyl-terminal end of the oligopeptide. The alpha-amino end of the amino acid is typically blocked with a t-butoxy-carbonyl group (Boc) or with a 9-fluorenylmethyloxycarbonyl (Fmoc) group to prevent the amino group which could otherwise react from participating in the coupling reaction. The side chain groups of the amino acids or analogs, if reactive, are also blocked (or protected) by various benzyl-derived protecting groups in the form of ethers, thioethers, esters, and carbamates.

The next step and subsequent repetitive cycles involve deblocking the amino-terminal (N-terminal) resin-bound amino acid (or terminal residue of the peptide chain) to remove the alpha-amino blocking group, followed by chemical addition (coupling) of the next blocked amino acid. This process is repeated for however many cycles are necessary to synthesize the entire molecule of interest. After each of the coupling and deblocking steps, the resin-bound molecule is thoroughly washed to remove any residual reactants before proceeding to the next. The solid support particles facilitate removal of reagents at any given step as the resin and resin-bound peptide can be readily filtered and washed while being held in a column or device with porous openings.

Synthesized molecules may be released from the resin by acid catalysis (typically with hydrofluoric acid or trifluoroacetic acid), which cleaves the molecule from the resin leaving an amide or carboxyl group on its C-terminal. Acidolytic cleavage also serves to remove the, protecting groups from the side chains of the amino acids in the synthesized peptide. Finished peptides can then be purified by any one of a variety of chromatography methods.

In accordance with a preferred embodiment, the peptides and peptide derivative mediators of RCT were synthesized by solid-phase synthesis methods with Na Fmoc chemistry. N^{a}-Fmoc protected amino acids and Rink amide MBHA resin and Wang resin were purchased from Novabiochem (San Diego, Calif.) or Chem-Impex Intl (Wood Dale, Ill.). Other chemicals and solvents were purchased from the following sources: trifluoroacetic acid (TFA), anisole, 1,2-ethanedithiol, thioanisole, piperidine, acetic anhydride, 2-Naphthoic acid and Pivaloic acid (Aldrich, Milwaukee, Wis.), HOBt and NMP (Chem-Impex Intl, Wood Dale, Ill.), dichloromethane, methanol and HPLC grade solvents from Fischer Scientific, Pittsburgh, Pa. The purity of the peptides was checked by LC/MS. The purification of the peptides was achieved using Preparative HPLC system (Agilent technologies, 1100 Series) on a C₁₈-bonded silica column (Tosoh Biospec preparative column, ODS-80TM, Dim: 21.5 mm×30cm). The peptides were eluted with a gradient system [50% to 90% of B solvent (acetonitrile:water 60:40 with 0.1% TFA)].

All peptides and analogs thereof were synthesized in a stepwise fashion via the solid-phase method, using Rink amide MBHA resin (0.5-0.66 mmol/g) or wang resin (1.2 mmol/g). The side chain's protecting groups were Arg (Pbf), Glu (OtBu) and Asp (OtBu). Each Fmoc-protected amino acid was coupled to this resin using a 1.5 to 3-fold excess of the protected amino acids. The coupling reagents were N-hydroxybenzotriazole (HOBt) and diisopropyl carbodiimide (DIC), and the coupling was monitored by Ninhydrin test. The Fmoc group was removed with 20% piperidine in NMP 30-60 minutes treatment and then successive washes with CH₂Cl₂, 10%TEA in CH₂Cl₂, Methanol and CH₂Cl₂. Coupling steps were followed by acetylation or with other capping groups as necessary.

A mixture of TFA, thioanisole, ethanedithiol and anisole (90:5:3:2, v/v) was used (4-5 hours at room temperature) to cleave the peptide from the peptide-resin and remove all of the side chain protecting groups. The crude peptide mixture was filtered from the sintered funnel, which was washed with TFA (2-3 times). The filtrate was concentrated into thick syrup and added into cold ether. The peptide precipitated as a white solid after keeping overnight in the freezer and centrifugation. The solution was decanted and the solid was washed thoroughly with ether. The resulting crude peptide was dissolved in buffer (acetonitrile:water 60:40 with 0.1% TFA) and dried. The crude peptide was purified by HPLC using preparative C-18 column (reverse phase) with a gradient system 50-90% B in 40 minutes [Buffer A: water containing 0.1% (v/v) TFA, Buffer B: Acetonitrile:water (60:40) containing 0.1% (v/v) TFA]. The pure fractions were concentrated over Speedvac. The yields varied from 5% to 20%.

Alternatively, the peptides of the preferred embodiments may be prepared by way of segment condensation, i.e., the joining together of small constituent peptide chains to form a larger peptide chain, as described, for example, in Liu et al., 1996, Tetrahedron Lett. 37(7):933-936; Baca, et al., 1995, J. Am. Chem. Soc. 117:1881-1887; Tam et al., 1995, Int. J. Peptide Protein Res. 45:209-216; Schnolzer and Kent, 1992, Science 256:221-225; Liu and Tam, 1994, J. Am. Chem. Soc. 116(10):4149-4153; Liu and Tam, 1994, PNAS. USA 91:6584-6588; Yamashiro and Li, 1988, Int. J. Peptide Protein Res. 31:322-334; Nakagawa et al., 1985, J. Am Chem. Soc. 107:7087-7083; Nokihara et al., 1989, Peptides 1988:166-168; Kneib-Cordonnier et al., 1990, Int. J. Pept. Protein Res. 35:527-538; the disclosures of which are incorporated herein in their entirety by reference thereto). Other methods useful for synthesizing the peptides of the preferred embodiments are described in Nakagawa et al., 1985, J. Am. Chem. Soc. 107:7087-7092.

For peptides produced by segment condensation, the coupling efficiency of the condensation step can be significantly increased by increasing the coupling time. Typically, increasing the coupling time results in increased racemization of the product (Sieber et al., 1970, Helv. Chim. Acta 53:2135-2150). Mediators of RCT containing N- and/or C-terminal blocking groups can be prepared using standard techniques of organic chemistry. For example, methods for acylating the N-terminus of a peptide or amidating or esterifying the C-terminus of a peptide are well-known in the art. Modes of carrying other modifications at the N- and/or C-terminus will be apparent to those of skill in the art, as will modes of protecting any side-chain functionalities as may be necessary to attach terminal blocking groups.

Likewise, for example, methods for deprotection of a protecting group on the N-terminus of a peptide or the C-terminus of a peptide are well-known in the art. Modes of carrying other modifications at the N- and/or C-terminus will be apparent to those of skill in the art, as will modes of deprotecting any side-chain functionalities as may be necessary to remove terminal blocking groups.

Pharmaceutically acceptable salts (counter ions) can be conveniently prepared by ion-exchange chromatography or other methods as are well known in the art.

### Pharmaceutical Formulations and Methods of Treatment

The mediators of RCT of the preferred embodiments can be used to treat any disorder in animals, especially mammals including humans, for which lowering serum cholesterol is beneficial, including without limitation conditions in which increasing serum HDL concentration, activating LCAT, and promoting cholesterol efflux and RCT is beneficial. Such conditions include, but are not limited to hyperlipidemia, and especially hypercholesterolemia, and cardiovascular disease such as atherosclerosis (including treatment and prevention of atherosclerosis) and coronary artery disease; restenosis (e.g., preventing or treating atherosclerotic plaques which develop as a consequence of medical procedures such as balloon angioplasty); and other disorders, such as ischemia, and endotoxemia, which often results in septic shock. The mediators of RCT can be used alone or in combination therapy with other drugs used to treat the foregoing conditions. Such therapies include, but are not limited to simultaneous or sequential administration of the drugs involved.

For example, in the treatment of hypercholesterolemia or atherosclerosis, the formulations of molecular mediators of RCT can be administered with any one or more of the cholesterol lowering therapies currently in use; e.g., bile-acid resins, niacin, and/or statins. Such a combined treatment regimen may produce particularly beneficial therapeutic effects since each drug acts on a different target in cholesterol synthesis and transport; i.e., bile-acid resins affect cholesterol recycling, the chylomicron and LDL population; niacin primarily affects the VLDL and LDL population; the statins inhibit cholesterol synthesis, decreasing the LDL population (and perhaps increasing LDL receptor expression); whereas the mediators of RCT affect RCT, increase HDL, increase LCAT activity and promote cholesterol efflux.

The mediators of RCT may be used in conjunction with fibrates to treat hyperlipidemia, hypercholesterolemia and/or cardiovascular disease such as atherosclerosis.

The mediators of RCT can be used in combination with the anti-microbials and anti-inflammatory agents currently used to treat septic shock induced by endotoxin.

The mediators of RCT can be formulated as molecule-based compositions or as molecule-lipid complexes which can be administered to subjects in a variety of ways, preferrably via oral administration, to deliver the mediators of RCT to the circulation. Exemplary formulations and treatment regimens are described below.

In another preferred embodiment, methods are provided for ameliorating and/or preventing one or more symptoms of hypercholesterolemia and/or atherosclerosis. The methods preferably involve administering to an organism, preferably a mammal, more preferably a human one or more of the compounds of the preferred embodiments (or mimetics of such compounds). The compound(s) can be administered, as described herein, according to any of a number of standard methods including, but not limited to injection, suppository, nasal spray, time-release implant, transdermal patch, and the like. In one particularly preferred embodiment, the compound(s) are administered orally (e.g. as a syrup, capsule, or tablet).

The methods involve the administration of a single compound of the preferred embodiments or the administration of two or more different compounds. The compounds can be provided as monomers or in dimeric, oligomeric or polymeric forms. In certain embodiments, the multimeric forms may comprise associated monomers (e.g. ionically or hydrophobically linked) while certain other multimeric forms comprise covalently linked monomers (directly linked or through a linker).

While the preferred embodiments are described with respect to use in humans, it is also suitable for animal, e.g. veterinary use. Thus preferred organisms include, but are not limited to humans, non-human primates, canines, equines, felines, porcines, ungulates, largomorphs, and the like.

The methods of the preferred embodiments are not limited to humans or non-human animals showing one or more symptom(s) of hypercholesterolemia and/or atherosclerosis (e.g., hypertension, plaque formation and rupture, reduction in clinical events such as heart attack, angina, or stroke, high levels of low density lipoprotein, high levels of very low density lipoprotein, or inflammatory proteins, etc.), but are useful in a prophylactic context. Thus, the compounds of the preferred embodiments (or mimetics thereof) may be administered to organisms to prevent the onset/development of one or more symptoms of hypercholesterolemia and/or atherosclerosis. Particularly preferred subjects in this context are subjects showing one or more risk factors for atherosclerosis (e.g., family history, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.). The preferred embodiments include the pharmaceutical formulations and the use of such preparations in the treatment of hyperlipidemia, hypercholesterolemia, coronary heart disease, atherosclerosis, diabetes, obesity, Alzheimer's Disease, multiple sclerosis, conditions related to hyperlipidemia, such as inflammation, and other conditions such as endotoxemia causing septic shock.

In one preferred embodiment, the molecular mediators of RCT can be synthesized or manufactured using any technique described herein pertaining to synthesis and purification of the mediators of RCT. Stable preparations which have a long shelf life may be made by lyophilizing the compoundseither to prepare bulk for reformulation, or to prepare individual aliquots or dosage units which can be reconstituted by rehydration with sterile water or an appropriate sterile buffered solution prior to administration to a subject.

In another preferred embodiment, the mediators of RCT may be formulated and administered in a molecule-lipid complex. This approach has some advantages since the complex should have an increased half-life in the circulation, particularly when the complex has a similar size and density to HDL, and especially the pre-β-1 or pre-β-2 HDL populations. The molecule-lipid complexes can conveniently be prepared by any of a number of methods described below. Stable preparations having a long shelf life may be made by lyophilization-the co-lyophilization procedure described below being the preferred approach. The lyophilized molecule-lipid complexes can be used to prepare bulk for pharmaceutical reformulation, or to prepare individual aliquots or dosage units which can be reconstituted by rehydration with sterile water or an appropriate buffered solution prior to administration to a subject.

A variety of methods well known to those skilled in the art can be used to prepare the molecule-lipid vesicles or complexes. To this end, a number of available techniques for preparing liposomes or proteoliposomes may be used. For example, the compound can be cosonicated (using a bath or probe sonicator) with appropriate lipids to form complexes. Alternatively the compound can be combined with preformed lipid vesicles resulting in the spontaneous formation of molecule-lipid complexes. In yet another alternative, the molecule-lipid complexes can be formed by a detergent dialysis method; e.g., a mixture of the compound, lipid and detergent is dialyzed to remove the detergent and reconstitute or form molecule-lipid complexes (e.g., see Jonas et al., 1986, Methods in Enzymol. 128:553-582).

While the foregoing approaches are feasible, each method presents its own peculiar production problems in terms of cost, yield, reproducibility and safety. In accordance with one preferred method, the compound and lipid are combined in a solvent system which co-solubilizes each ingredient and can be completely removed by lyophilization. To this end, solvent pairs should be carefully selected to ensure co-solubility of both the amphipathic compound and the lipid. In one embodiment, compound(s) or derivatives/analogs thereof, to be incorporated into the particles can be dissolved in an aqueous or organic solvent or mixture of solvents (solvent 1). The (phospho)lipid component is dissolved in an aqueous or organic solvent or mixture of solvents (solvent 2) which is miscible with solvent 1, and the two solutions are mixed. Alternatively, the compound and lipid can be incorporated into a co-solvent system; i.e., a mixture of the miscible solvents. A suitable proportion of compound to lipids is first determined empirically so that the resulting complexes possess the appropriate physical and chemical properties; i.e., usually (but not necessarily) similar in size to HDL. The resulting mixture is frozen and lyophilized to dryness. Sometimes an additional solvent must be added to the mixture to facilitate lyophilization. This lyophilized product can be stored for long periods and will remain stable.

The lyophilized product can be reconstituted in order to obtain a solution or suspension of the molecule-lipid complex. To this end, the lyophilized powder may be rehydrated with an aqueous solution to a suitable volume (often 5 mgs compound/ml which is convenient for intravenous injection). In a preferred embodiment the lyophilized powder is rehydrated with phosphate buffered saline or a physiological saline solution. The mixture may have to be agitated or vortexed to facilitate rehydration, and in most cases, the reconstitution step should be conducted at a temperature equal to or greater than the phase transition temperature of the lipid component of the complexes. Within minutes, a clear preparation of reconstituted lipid-protein complexes results.

An aliquot of the resulting reconstituted preparation can be characterized to confirm that the complexes in the preparation have the desired size distribution; e.g., the size distribution of HDL. Gel filtration chromatography can be used to this end. For example, a Pharmacia Superose 6 FPLC gel filtration chromatography system can be used. The buffer used contains 150 mM NaCl in 50 mM phosphate buffer, pH 7.4. A typical sample volume is 20 to 200 microliters of complexes containing 5 mgs compound/ml. The column flow rate is 0.5 mls/min. A series of proteins of known molecular weight and Stokes' diameter as well as human HDL are preferably used as standards to calibrate the column. The proteins and lipoprotein complexes are monitored by absorbance or scattering of light of wavelength 254 or 280 nm.

The mediators of RCT of the preferred embodiments can be complexed with a variety of lipids, including saturated, unsaturated, natural and synthetic lipids and/or phospholipids. Suitable lipids include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, sphingomyelin, sphingolipids, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, dipalmitoylsphingomyelin, distearoylsphingomyelin, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives.

The pharmaceutical formulation of the preferred embodiments contain the molecular mediators of RCT or the molecule-lipid complex as the active ingredient in a pharmaceutically acceptable carrier suitable for administration and delivery in vivo. As the compounds may contain acidic and/or basic termini and/or side chains, the compounds can be included in the formulations in either the form of free acids or bases, or in the form of pharmaceutically acceptable salts.

Injectable preparations include sterile suspensions, solutions or emulsions of the active ingredient in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives.

Alternatively, the injectable formulation may be provided in powder form for reconstitution with a suitable vehicle, including but not: limited to sterile pyrogen free water, buffer, dextrose solution, etc., before use. To this end, the mediators of RCT may be lyophilized, or the co-lyophilized molecule-lipid complex may be prepared. The stored preparations can be supplied in unit dosage forms and reconstituted prior to use in vivo.

For prolonged delivery, the active ingredient can be formulated as a depot preparation, for administration by implantation; e.g., subcutaneous, intradermal, or intramuscular injection. Thus, for example, the active ingredient may be formulated with suitable polymeric or hydrophobic materials (e.g., as an emulsion in an acceptable oil) or ion exchange resins, or as sparingly soluble derivatives; e.g., as a sparingly soluble salt form of the mediators of RCT.

Alternatively, transdermal delivery systems manufactured as an adhesive disc or patch which slowly releases the active ingredient for percutaneous absorption may be used. To this end, permeation enhancers may be used to facilitate transdermal penetration of the active ingredient. A particular benefit may be achieved by incorporating the mediators of RCT of the preferred embodiments or the molecule-lipid complex into a nitroglycerin patch for use in patients with ischemic heart disease and hypercholesterolemia.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration, the compositions may take the form of tablets or lozenges formulated in conventional manner. For rectal and vaginal routes of administration, the active ingredient may be formulated as solutions (for retention enemas) suppositories or ointments.

For administration by inhalation, the active ingredient can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The molecule mediators of RCT and/or molecule-lipid complexes of the preferred embodiments may be administered by any suitable route that ensures bioavailability in the circulation. This can be achieved by parenteral routes of administration, including intravenous (IV), intramuscular (IM), intradermal, subcutaneous (SC) and intraperitoneal (IP) injections. However, other routes of administration may be used. For example, absorption through the gastrointestinal tract can be accomplished by oral routes of administration (including but not limited to ingestion, buccal and sublingual routes) provided appropriate formulations (e.g., enteric coatings) are used to avoid or minimize degradation of the active ingredient, e.g., in the harsh environments of the oral mucosa, stomach and/or small intestine. Oral administration has the advantage of easy of use and therefore enhanced compliance. Alternatively, administration via mucosal tissue such as vaginal and rectal modes of administration may be utilized to avoid or minimize degradation in the gastrointestinal tract. In yet another alternative, the formulations of the preferred embodiments can be administered transcutaneously (e.g., transdermally), or by inhalation. It will be appreciated that the preferred route may vary with the condition, age and compliance of the recipient.

The actual dose of molecular mediators of RCT or molecule-lipid complex used will vary with the route of administration, and should be adjusted to achieve circulating plasma concentrations of 1.0 mg/l to 2 g/l. Data obtained in animal model systems described herein show that the ApoA-I agonists of the preferred embodiments associate with the HDL component, and have a projected half-life in humans of about five days. Thus, in one embodiment, the mediators of RCT can be administered by injection at a dose between 0.5 mg/kg to 100 mg/kg once a week. In another embodiment, desirable serum levels may be maintained by continuous infusion or by intermittent infusion providing about 0.1 mg/kg/hr to 100 mg/kg/hr.

Toxicity and therapeutic efficacy of the various mediators of RCT can be determined using standard pharmaceutical procedures in cell culture or experimental animals for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. ApoA-I molecular agonists which exhibit large therapeutic indices are preferred.

Other Uses

The mediators of RCT agonists of the preferred embodiments can be used in assays in vitro to measure serum HDL, e.g., for diagnostic purposes. Because the mediators of RCT associate with the HDL and LDL component of serum, the agonists can be used as “markers” for the HDL and LDL population. Moreover, the agonists can be used as markers for the subpopulation of HDL that are effective in RCT. To this end, the agonist can be added to or mixed with a patient serum sample; after an appropriate incubation time, the HDL component can be assayed by detecting the incorporated mediators of RCT. This can be accomplished using labeled agonist (e.g., radiolabels, fluorescent labels, enzyme labels, dyes, etc.), or by immunoassays using antibodies (or antibody fragments) specific for the agonist.

Alternatively, labeled agonist can be used in imaging procedures (e.g., CAT scans, MRI scans) to visualize the circulatory system, or to monitor RCT, or to visualize accumulation of HDL at fatty streaks, atherosclerotic lesions, etc. (where the HDL should be active in cholesterol efflux).

### Assays For Analysis of Mediators of Reverse Cholesterol Transport

LCAT Activation Assay

The mediators of RCT in accordance with preferred embodiments can be evaluated for potential clinical efficacy by various in vitro assays, for example, by their ability to activate LCAT in vitro. In the LCAT assay, substrate vesicles (small unilamellar vesicles or “SUVs”) composed of egg phophatidylcholine (EPC) or 1-palmitoyl-2-oleyl-phosphatidyl-choline (POPC) and radiolabelled cholesterol are preincubated with equivalent masses either of compound or ApoA-I (isolated from human plasma). The reaction is initiated by addition of LCAT (purified from human plasma). Native ApoA-I, which was used as positive control, represents 100% activation activity. “Specific activity” (i.e., units of activity (LCAT activation)/unit of mass) of the molecular mediators can be calculated as the concentration of mediator that achieves maximum LCAT activation. For example, a series of concentrations of the compound (e.g., a limiting dilution) can be assayed to determine the “specific activity” for the compound—the concentration which achieves maximal LCAT activation (i.e., percentage conversion of cholesterol to cholesterol ester) at a specific timepoint in the assay (e.g., 1 hr.). When plotting percentage conversion of cholesterol at, e.g., 1 hr., against the concentration of compound used, the “specific activity” can be identified as the concentration of compound that achieves a plateau on the plotted curve.

Preparation of Substrate Vesicles

The vesicles used in the LCAT assay are SUVs composed of egg phosphatidylcholine (EPC) or 1-palmitoyl-2-oleyl-phosphatidylcholine (POPC) and cholesterol with a molar ratio of 20:1. To prepare a vesicle stock solution sufficient for 40 assays, 7.7 mg EPC (or 7.6 mg POPC; 10 μmol), 78 μg (0.2 μmol) 4−¹⁴ C-cholesterol, 116 μg cholesterol (0.3 μmol) are dissolved in 5 ml xylene and lyophilized. Thereafter 4 ml of assay buffer is added to the dry powder and sonicated under nitrogen atmosphere at 4° C. Sonication conditions: Branson 250 sonicator, 10 mm tip, 6×5 minutes; Assay buffer: 10 mM Tris, 0.14 M NaCl, 1 mM EDTA, pH 7.4. The sonicated mixture is centrifuged 6 times for 5 minutes each time at 14,000 rpm (16,000×g) to remove titanium particles. The resulting clear solution is used for the enzyme assay.

Purification of LCAT

For the LCAT purification, dextran sulfate/Mg²+ treatment of human plasma is used to obtain lipoprotein deficient serum (LPDS), which is sequentially chromatographed on Phenylsepharose, Affigelblue, ConcanavalinA sepharose and anti-ApoA-I affinity chromatography.

Preparation of LPDS

To prepare LPDS, 500 ml plasma is added to 50 ml dextran sulfate (MW=500,000) solution. Stir 20 minutes. Centrifuge for 30 minutes at 3000 rpm (16,000×g) at 4° C. Use supernatant (LPDS) for further purification (ca. 500 ml).

Phenylsepharose Chromatography

The following materials and conditions were used for the phenylsepharose chromatography. Solid phase: phenylsepharose fast flow, high subst. grade, Pharmaciacolunm: XK26/40, gel bed height: 33 cm, V=ca, 175 mlflow rates: 200 ml/hr (sample)wash: 200 ml/hr (buffer)elution: 80 ml/hr (distilled water)buffer: 10 mM Tris, 140 mM NaCl, 1 mM EDTA pH 7.4, 0.01% sodium azide.

Equilibrate the column in Tris-buffer, add 29 g NaCl to 500 ml LPDS and apply to the column. Wash with several volumes of Tris buffer until the absorption at 280 nm wavelength is approximately at the baseline, then start the elution with distilled water. The fractions containing protein are pooled (pool size: 180 ml) and used for Affigelblue chromatography.

Affigelblue Chromatography

The phenylsepharose pool is dialyzed overnight at 4° C. against 20 mM Tris-HCl, pH7.4, 0.01% sodium azide. The pool volume is reduced by ultrafiltration (Amicon YM30) to 50-60 ml and loaded on an Affigelblue column. Solid phase: Affigelblue, Biorad, 153-7301 column, XK26/20, gel bed height: ca. 13 cm; column volume: approx. 70 ml. Flow rates: loading: 15 ml/h wash: 50 ml/h. Equilibrate column in Tris-buffer. Apply phenylsepharose pool to column. Start in parallel to collect fractions. Wash with Tris-buffer. The pooled fractions (170 ml) were used for ConA chromatography.

ConA Chromatography

The Affigelblue pool was reduced via Amicon (YM30) to 30-40 ml and dialyzed against ConA starting buffer (1 mM Tris HCl pH7.4; 1 mM MgCl₂, 1 mM MnCl₂, 1 mM CaCl₂, 0.01% sodium azide) overnight at 4° C. Solid phase: ConA sepharose (Pharmacia) column: XK26/20, gel bed height: 14 cm (75 ml). Flow rates: loading 40 ml/h washing (with starting buffer): 90 ml/h elution: 50 ml/h, 0.2M Methyl-α-D-mannoside in 1 mM Tris, pH 7.4. The protein fractions of the mannoside elutions were collected (110 ml), and the volume was reduced by ultrafiltration (YM30) to 44 ml. The ConA pool was divided in 2 ml aliquots, which are stored at −20° C.

Anti-ApoA-I Affinity Chromatography

Anti-ApoA-I affinity chromatography was performed on Affigel-Hz material (Biorad), to which the anti-ApoA-I abs have been coupled covalently. Column: XK16/20, V=16 ml. The column was equilibrated with PBS pH 7.4. Two ml of the ConA pool was dialyzed for 2 hours against PBS before loading onto the column. Flow rates: loading: 15 ml/hour washing (PBS) 40 ml/hour. The pooled protein fractions (V=14 ml) are used for LCAT assays. The column is regenerated with 0.1 M. Citrate buffer (pH 4.5) to elute bound A-I (100 ml), and immediately after this procedure reequilibrated with PBS.

Pharmacokinetics of the Mediators of RCT

The following experimental protocols can be used to demonstrate that the mediators of RCT are stable in the circulation and associate with the HDL component of plasma.

Synthesis and/or Radiolabeling of Compound Agonists

The ¹²⁵I-labeled LDL was prepared by the iodine monochloride procedure to a specific activity of 500-900 cpm/ng (Goldstein and Brown 1974 J. Biol. Chem. 249:5153-5162). Binding and degradation of low density lipoproteins by cultured human fibroblasts were determined at final specific activities of 500-900 cpm/ng as described (Goldstein and Brown 1974 J. Biol. Chem. 249:5153-5162). In every case, >99% radioactivity was precipitable by incubation of the lipoproteins at 4° C. with 10% (wt/vol) trichloroacetic acid (TCA). The Tyr residue was attached to N-Terminus of each compound to enable its radioiodination. The compounds were radioiodinated with Na¹²⁵I(ICN), using lodo-Beads (Pierce Chemicals) and following the manufacturer's protocol, to a specific activity of 800-1000 cpm/ng. After dialysis, the precipitable radioactivity (10% TCA) of the compounds was always >97%.

Alternatively, radiolabeled compounds could be synthesized by coupling ¹⁴C-labeled Fmoc-Pro as the N-terminal amino acid. L-[U-¹⁴ C]X, specific activity 9.25 GBq/mmol, can be used for the synthesis of labeled agonists containing X. The synthesis may be carried out according to Lapatsanis, Synthesis, 1983, 671-173. Briefly, 250 μM (29.6 mg) of unlabeled L-X is dissolved in 225 μl of a 9% Na₂ CO₃ solution and added to a solution (9% Na₂CO₃) of 9.25 MBq (250 μM) ¹⁴C-labeled L-X. The liquid is cooled down to 0° C., mixed with 600 μM (202 mg) 9-fluorenylmethyl-N-succinimidylcarbonate (Fmoc-OSu) in 0.75 ml DMF and shaken at room temperature for 4 hr. Thereafter, the mixture is extracted with Diethylether (2×5 ml) and chloroform (1×5 ml), the remaining aqueous phase is acidified with 30% HCl and extracted with chloroform (5×8 ml). The organic phase is dried over Na₂ SO₄₁ filtered off and the volume is reduced under nitrogen flow to 5 ml. The purity was estimated by TLC (CHCl₃ :MeOH:Hac, 9:1:0.1 v/v/v, stationary phase HPTLC silicagel 60, Merck, Germany) with UV detection, e.g., radiochemical purity:Linear Analyzer, Berthold, Germany; reaction yields may be approximately 90% (as determined by LSC).

The chloroform solution containing ¹⁴C-compound X is used directly for synthesis. A resin containing amino acids 2-22, can be synthesized automatically as described above and used for the synthesis. The sequence of the peptide is determined by Edman degradation. The coupling is performed as previously described except that HATU (O-(7-azabenzotriazol-1-yl)1-, 1,3,3-tetramethyluroniumhexafluorophosphate) is preferably used instead of TBTU. A second coupling with unlabeled Fmoc-L-X is carried out manually.

Pharmacokinetics in Mice

In each experiment, 300-500 μg/kg (0.3-0.5 mg/kg) [or more such as 2.5 mg/k] radiolabeled compound may be injected intraperitoneally into mice which were fed normal mouse chow or the atherogenic Thomas-Harcroft modified diet (resulting in severely elevated VLDL and BDL cholesterol). Blood samples are taken at multiple time intervals for assessment of radioactivity in plasma.

Stability in Human Serum

100 μg of labeled compound may be mixed with 2 ml of fresh human plasma (at 37° C.) and delipidated either immediately (control sample) or after 8 days of incubation at 37° C. (test sample). Delipidation is carried out by extracting the lipids with an equal volume of 2:1 (v/v) chloroform:methanol. The samples are loaded onto a reverse-phase C₁₈ HPLC column and eluted with a linear gradient (25-58% over 33 min) of acetonitrile (containing 0.1% w TFA). Elution profiles are followed by absorbance (220 nm) and radioactivity.

Formation of Pre-β Like Particles

Human HDL may be isolated by KBr density ultra centrifugation at density d=1.21 g/ml to obtain top fraction followed by Superose 6 gel filtration chromatography to separate HDL from other lipoproteins. Isolated HDL is adjusted to a final concentration of 1.0 mg/ml with physiological saline based on protein content determined by Bradford protein assay. An aliquot of 300 μl is removed from the isolated HDL preparation and incubated with 100 μl labeled compound (0.2-1.0 μg/μl) for two hours at 37° C. Multiple separate incubations are analyzed including a blank containing 100 μl physiological saline and four dilutions of labeled compound. For example: (i) 0.20 μg/μl compound:HDL ratio=1:15; (ii) 0.30 μg/μl compound:HDL ratio=1:10; (iii) 0.60 μg/μl compound:HDL ratio=1:5; and (iv) 1.00 μg/μl compound:HDL ratio=1:3. Following the two hour incubation, a 200 μl aliquot of the sample (total volume=400 μl) is loaded onto a Superose 6 gel filtration column for lipoprotein separation and analysis and 100 μl is used to determine total radioactivity loaded.

Association of Mediators With Human Lipoproteins

The association of molecular mediators with human lipoprotein fractions can be determined by incubating labeled compound with each lipoprotein class (HDL, LDL and VLDL) and a mixture of the different lipoprotein classes. HDL, LDL and VLDL are isolated by KBr density gradient ultracentrifugation at d=1.21 g/ml and purified by FPLC on a Superose 6B column size exclusion column (chromatography is carried out with a flow rate of 0.7 ml/min and a running buffer of 1 mM Tris (pH 8), 115 mM NaCl, 2 mM EDTA and 0.0% NaN₃). Labeled compound is incubated with HDL, LDL and VLDL at a compound:phospholipid ratio of 1:5 (mass ratio) for 2 h at 37° C. The required amount of lipoprotein (volumes based on amount needed to yield 1000 μg) is mixed with 0.2 ml of compound stock solution (1 mg/ml) and the solution is brought up to 2.2 ml using 0.9% of NaCl.

After incubating for 2 hr at 37° C., an aliquot (0.1 ml) is removed for determination of the total radioactivity (e.g., by liquid scintilation counting or gamma counting depending on labeling isotope), the density of the remaining incubation mixture is adjusted to 1.21 g/ml with KBr, and the samples centrifuged at 100,000 rpm (300,000 g) for 24 hours at 4° C. in a TLA 100.3 rotor using a Beckman tabletop ultracentrifuge. The resulting supernatant is fractionated by removing 0.3 ml aliquots from the top of each sample for a total of 5 fractions, and 0.05 ml of each fraction is used for counting. The top two fractions contain the floating lipoproteins, the other fractions (3-5) correspond to compound in solution.

Selective Binding to HDL Lipids

Human plasma (2 ml) is incubated with 20, 40, 60, 80, and 100 μg of labeled compound for 2 hr at 37° C. The lipoproteins are separated by adjusting the density to 1.21 g/ml and centrifugation in TLA 100.3 rotor at 100,000 rpm (300,000 g) for 36 hr at 40° C. The top 900 μl (in 300 μl fractions) is taken for the analysis. 50 μl from each 300 μl fraction is counted for radioactivity and 200 μl from each fraction is analyzed by FPLC (Superose 6/Superose 12 combination column).

Use of the Mediators of Reverse Cholesterol Transport in Animal Model Systems

The efficacy of the mediators of RCT of the preferred embodiments can be demonstrated in rabbits or other suitable animal models.

Preparation of the Phospholipid/Compound Complexes

Small discoidal particles consisting of phospholipid (DPPC) and compound are prepared following the cholate dialysis method. The phospholipid is dissolved in chloroform and dried under a stream of nitrogen. The compound is dissolved in buffer (saline) at a concentration of 1-2 mg/ml. The lipid film is redissolved in buffer containing cholate (43° C.) and the compound solution is added at a 3:1 phospholipid/compound weight ratio. The mixture is incubated overnight at 43° C. and dialyzed at 43° C. (24 hr), room temperature (24 hr) and 4° C. (24 hr), with three changes of buffer (large volumes) at temperature point. The complexes may be filter sterilized (0.22 μm) for injection and storage at 4° C.

Isolation and Characterization of the Compound/Phospholipid Particles

The particles may be separated on a gel filtration column (Superose 6 HR). The position of the peak containing the particles is identified by measuring the phospholipid concentration in each fraction. From the elution volume, the Stokes radius can be determined. The concentration of compound in the complex is determined by measuring the phenylalanine content (by HPLC) following a 16 hr acid hydrolysis.

Injection in the Rabbit

Male New Zealand White rabbits (2.5-3 kg) are injected intravenously with a dose of phospholipid/compound complex (5 or 10 mg/kg bodyweight, expressed as compound) in a single bolus injection not exceeding 10-15 ml. The animals are slightly sedated before the manipulations. Blood samples (collected on EDTA) are taken before and 5, 15, 30, 60, 240 and 1440 minutes after injection. The hematocrit (Hct) is determined for each sample. Samples are aliquoted and stored at −20° C. before analysis.

Analysis of the Rabbit Sera

The total plasma cholesterol, plasma triglycerides and plasma phospholipids are determined enzymatically using commercially available assays, for example, according to the manufacturer's protocols (Boehringer Mannheim, Mannheim, Germany and Biomerieux, 69280, Marcy-L'etoile, France).

The plasma lipoprotein profiles of the fractions obtained after the separation of the plasma into its lipoprotein fractions may be determined by spinning in a sucrose density gradient. For example, fractions are collected and the levels of phospholipid and cholesterol can be measured by conventional enzymatic analysis in the fractions corresponding to the VLDL, ILDL, LDL and HDL lipoprotein densities.

### Synthesis of RCT Mediators Bearing Modified Amino Acids or Molecular Group Bioisosteres or Functional Group Bioisosteres

Synthesis of Lipophilic Group Modified Peptide Sequence Based on Atorvastatin General Analytical Methods.

All reagents were of commercial quality. Solvents were dried and purified by standard methods. Amino acid derivatives were obtained from commercial sources. Analytical TLC was performed on aluminum sheets coated with a 0.2 mm layer of silica gel 60 F₂₅₄, Merck, and preparative TLC was performed on 20 cm×20 cm glass plates coated with a 2 mm layer of silica gel PF₂₅₄, Merck. Silica gel 60 (230-400 mesh), Merck, was used for flash chromatography. Melting points were taken on a micro-hot-stage apparatus and are uncorrected. ¹H NMR spectra were recorded with Brucker 400 spectrometer, operating at 400 MHz, using TMS or solvent as reference. Elemental analyses were carried out at NuMega Resonance Laboratories, San Diego. Preparative reverse-phase HPLC (Glison) of the final products was performed on a Phenomenex Luna 5μ C₁₈ (2) (60 mm×21.2 mm) column with a flow rate of 15 mL/min, using a tunable UV detector set at 254 nm. Mixtures of CH₃CN and H₂O were used as mobile phases in gradient mode (CH₃CN=5% -95%). Analysis by LC/UV/ELSD/MS was performed using an API 150 EX instrument from PE Sciex. ESI-MS experiments were performed, in positive mode.

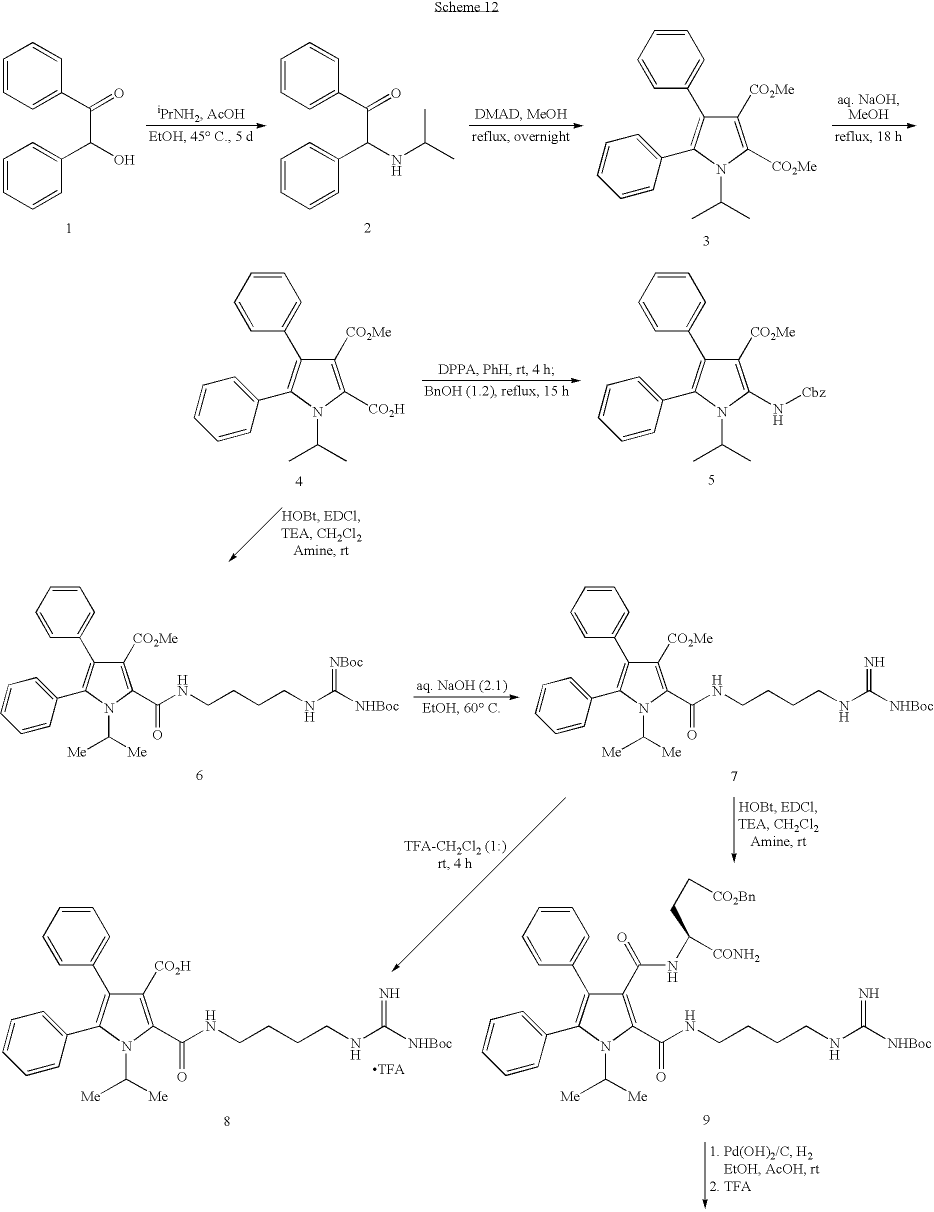

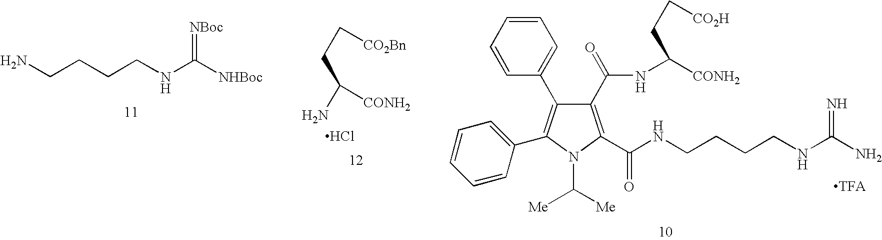

To a solution of benzoin (8.0 g, 37.7 mmol) in EtOH (150 mL) was added isopropylamine (2.45 g, 41.5 mmol), followed by glacial AcOH (few drops). The reaction was heated at 45° C. for 5 d. The volatile materials were removed in a rotary evaporator and dried in vacuo. The crude material was used in the following reaction.

Dimethyl 1-isopropyl-4,5-diphenyl-1H-pnrrole-2,3-dicarboxylate (3)

Dimethyl acetylenedicarboxylate (DMAD, 7.0 g, 57 mmol) was added to the above amine 2 (8.0 g, 32 mmol) in MeOH (100 mL) and the reaction was heated at reflux overnight under argon. The reaction mixture was cooled in an ice-bath and filtered. The solids were washed with cold MeOH (20 mL) and dried to furnish pyrrole 3 as white powder (9.8 g, 82%).

3 -(Methoxycarbonyl)-1-isopropyl-4,5-diphenyl-1H-pyrrole-2-carboxylic Acid (4)

To the diester 2 (6.12 g, 16.1 mmol) were added MeOH (100 mL) and 1 M NaOH (aq., 17.05 mL). The mixture was heated at reflux for 18 h. The volatiles were removed in rotary evaporator. The residue was taken up in water (100 mL) and extracted with ether (2×50 mL) and kept aside to obtain unreacted starting material. The aqueous phase was acidified with 4 M HCl to pH ˜3 and extracted with ether (3×60 mL), washed with water (50 mL) and dried (Na₂SO₄). After evaporation and drying, the monoacid 4 was obtained (5.02 g, 85%) as white solid.

Benzyl 3-(methoxycarbonyl)-1-isopropyl-4,5-diphenyl-1H-pyrrol-2-ylcarbamate (5)

To a solution of the above monoacid 4 (o.1 g, 0.27 mmol) in benzene (3 mL), triethylamine (45 μL, 0.33 mmol) and diphenylphosporylazide (DPPA, 0.091 g, 0.33 mmol) were added and stirred at rt for 4 h. Then benzyl alcohol (35 μL, 33 mmol) was added and heated the reaction mixture at reflux for 15 h. The reaction was allowed to cool to rt and 5 % NaHCO₃ (5 mL) was added and extracted with ether (2×10 mL). Upon concentration, it gave the carbamate 5.

Methyl 2-(4-(N,N′-di(Boc)guanidinyl)butylcarbamoyl)-1-isopropyl-4,5-diphenyl-1H-pyrrole-3-carboxylate (6)

To a solution of the acid 4 (0.1 g, 0.27 mmol) in CH₂Cl₂ (3 mL), EDCI (0.053 g, 0.27 mmol), HOBt (0.037 g, 0.27 mmol), Et₃N (38 μL, 0.27 mmol) and amine 11 (0.086 g, 0.26 mol) were added in that order and stirred at rt overnight. The reaction was diluted with CH₂Cl₂ (10 ml) and washed with satd. NaHCO₃ (5 mL), brine (5 mL) and dried (Na₂SO₄) to obtain amide 6 (0.165 g, 88.8%) as white solid.

2-(4-(N(Boc)guanidinyl)butylcarbamoyl)-1-isopropyl-4,5-diphenyl-1H-pyrrole-3-carboxylic acid (7)

To the ester 6 (0.16 g, 0.237 mmol) were added MeOH (10 mL) and 1 M NaOH (aq., 1.0 mL). The mixture was heated at reflux for 18 h. The volatiles were removed in rotary evaporator. The residue was taken up in water (10 mL) acidified with 4 M HCl to pH ˜3 and extracted with ether (3×10 mL), washed with water (10 mL) and dried (Na₂SO₄). After evaporation and drying, the acid 7 was obtained (0.121 g, 91%).

2-(4-(Guanidinyl)butylcarbamoyl)-1-isopropyl-4,5-diphenyl-1H-pyrrole-3-carboxylic acid.TFA (8)

To a solution of the above Boc-protected compound 7 (0.10 g, 0.178 mmol) in CH₂Cl₂ (3 mL) was added trifluoroacetic acid (3 mL) and stirred at rt for 4 h. The volatiles were removed in a rotary evaporator. Reverse-phase chromatography (CH₃CN—H₂O/0.1% TFA) of the crude gave the desired product 8 (0.075 g, 91 %) as trifluoroacetic acid salt.

(S)-(4-(N3-(1 -carbamoyl-3-carbobenzyloxypropyl)-1-isopropyl-4,5-diphenyl-1H-pyrrole-2,3-dicarboxamido)butyl)-N-(Boc)guanidine (9)

To a solution of the acid 7 (0.132 g, 0.23 mmol) in CH₂Cl₂ (10 mL), EDCI (0.051 g, 0.23 mmol), HOBt (0.032 g, 0.23 mmol), Et₃N (65 μL, 0.23 mmol) and amine 12 (0.061 g, 0.22 mol) were added in that order and stirred at rt overnight. The reaction was diluted with CH₂Cl₂ (10 ml) and washed with satd. NaHCO₃ (5 mL), brine (5 mL) and dried (Na₂SO₄) to obtain the white solid amide 6 (0.127 g, 69 %).

(S)-(4-(N3-(1 -carbamoyl-3-carboxypropyl)-1-isopropyl-4,5-diphenyl-1H-pyrrole-2,3-dicarboxamido)butyl)guanidine.TFA (10)

To a solution of the benzyl ester 9 (0.02 g, 0.025 mmol) in EtOH (10 mL), acetic acid (0.1 mL) and 10 % Pd(OH)₂/C (0.01 g) were added and stirred at rt under hydrogen (balloon). After overnight stirring, the reaction was filtered, washed with EtOH and evaporated to obtain crude, which was taken in TFA (2 mL) and stirred at rt for 4 h. Upon evaporation and purification by reverse-phase chromatography (CH₃CN—H₂O/0.1 % TFA) the desired product was obtained as trifluoroacetic acid salt.
Synthesis of Lipophilic Group Modified Peptide Sequence Based on Nisvastatin 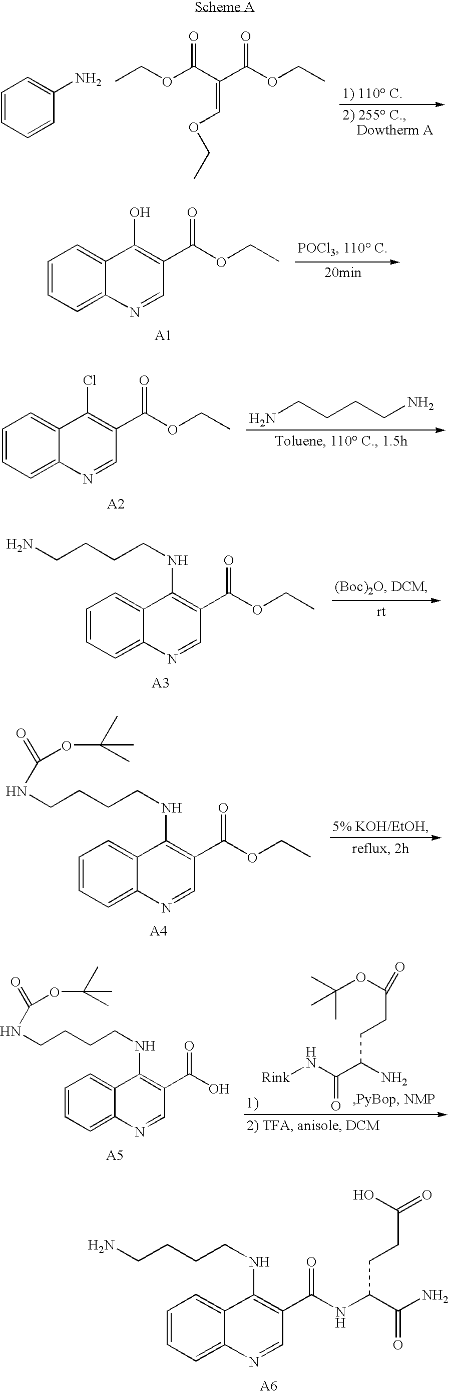
Scheme A
Ethyl 4-hydroxyguinoline-3-carboxylate (A1)

Aniline (2.15 g, 23 mmol) and diethyl ethoxymethylene malonate (5 g, 23 mmol) were mixed neat and heated at 110° C. for 2 h then cooled and allowed to stand at room temperature for 15 h. During this time the reaction mixture crystallized.

Dowtherm A (70 mL) was heated to 255° C. and the melted crystals were added and the mixture heated at 255° C. for 20 min. The mixture was then poured into a stainless steel container cooled to 0° C. with an ice bath. Hexanes were added to the cold solution to precipitate the product which was collected by filtration and rinsed with another portion of hexanes. The product was recrystallized from EtOH to give the product as a white solid. (1.6 g, 7.3 mmol, 32%, M.P. 309C) that was used without further purification in the next step.

Ethyl 4-chloroquinoline-3-carboxylate (A2)

To solid ethyl 4-hydroxyquinoline-3-carboxylate (A1) (1.5g, 7mmol) was added POCl₃ (2.2 g, 1.3 mL, 14 mmol) and the mixture heated at 110° C. for 20 min. The mixture was poured into NH₃ (aq, 28-30%) and ice and then stirred until granular. The melted ice mixture was extracted with ether (3×40 mL) and the combined organic layers dried (MgSO₄), filtered, and concentrated to give the product as an oil that crystallized on standing (1.44 g, 6 mmol, 87%) that was used as is without further purification.

Ethyl 4-(4-aminobutylamino)guinoline-3-carboxylate (A3)

To a solution of ethyl 4-chloroquinoline-3-carboxylate (A2) (0.5 g, 2.1 mmol) in toluene (10 mL) was added diaminobutane (10×, 1.85 g, 21 mmol) and the mixture heated at 110° C. for 1.5 h. During this time a salt formed that was removed by filtration while hot and the filtrate concentrated under reduced pressure to give an oil. Water was added and the mixture extracted with DCM (2×25 mL). The combined organic layers were dried (MgSO₄), filtered and concentrated to give an oil that crystallized on standing (476 mg, 1.66 mmol, 79%) that was used in subsequent steps without further purification.

tert-Butyl 4-(3-(ethoxycarbonyl)quinolin-4-ylamino)butylcarbamate (A4)

To a solution of ethyl 4-(4-aminobutylamino)quinoline-3-carboxylate (A3) in DCM (60 mL) was added di-tert-butyl dicarbonate and the mixture stirred at room temperature for 8 h. The mixture was washed with 2M Na₂CO₃ (20 mL), water (20 mL), sat. NaCl (20 mL), dried (MgSO₄), filtered, and concentrated to give the product as a yellow oil (4 g) that was used as is in the subsequent step.

4-(4-tert-Butoxycarbonylamine-butylamino)-gunoline-3-carboxylic acid (A5)

A solution of tert-Butyl 4-(3-(ethoxycarbonyl)quinolin-4-ylamino)butylcarbamate (A4) in ethanolic KOH (5%, 100 mL) was refluxed for 2 h and then concentrated under reduced pressure. The residue was dissolved in water (25 mL) and HCl (20%) used to adjust the resulting mixture to pH-8. A solid appeared and was collected by filtration and the resulting cake washed with water and dried under vacuum to give the product as a white powder (2.763 g) that was used in the next step.

4-{[4-(4-Aminobutylamino)-guinoline-3-carbonyl]-amino}-4-carbamoyl-butyric acid (A6)

D-Glutamic acid tertbutyl ester bound to rink amide MBHA resin (2 g, 1.32 mmol), 4-(4-tert-Butoxycarbonylamine-butylamino)-qunoline-3-carboxylic acid (A5) (2eq, 950 mg, 2.64 mmol), and PyBop (1.4 g, 2.64 mmol) were added to a flame dried 50mL round bottomed flask. NMP (25 mL) was added and the solution stirred for 18 h at room temperature. The mixture was filtered and rinsed successively with DCM, MeOH alternating 3× each and air dried. The resulting beads were suspended in TFA (10 mL) and anisole added (0.2 mL) and stirred at room temperature for 1 h. The solid was filtered off and the filtrate concentrated under reduced pressure to give an oil. Purification using reverse phase HPLC using ACN/H₂O/0.1%TFA (gradient from 5% to 95% ACN) gave the product as a white solid after lyophillization (127 mg, 0.33 mmol, 13%). MP 108° C., ¹H NMR (400MHz) δ8.96 (d, J=7.6Hz, 1H), 8.72 (br s, 1H), 8.58 (d, J=8.4Hz, 1H), 7.94 (m, 2H), 7.71 (m, 4H), 7.63 (s, 1H), 7.18 (s, 1H), 4.35 (m; 1H), 2.81 (br s, 2H), 2.37 (m, 2H), 2.07-2.00 (series of m, 2H), 1.75 (m, 2H), 1.60 (m, 2H) EIMS m/z M⁺¹ 388.7. Anal. C₁₉H₂₅N₅O₄+2 TFA+2 H₂O 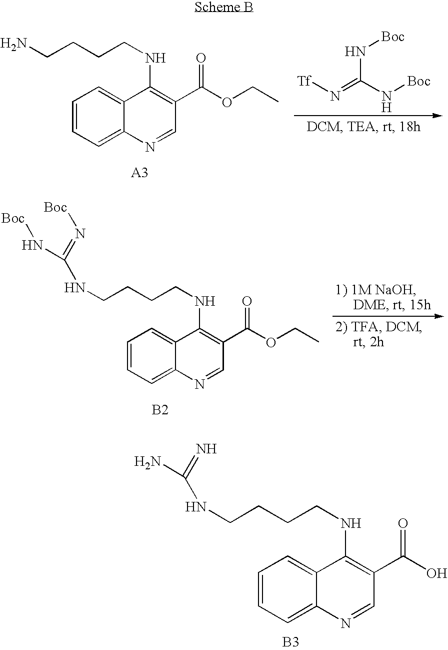
Scheme B
4-(4-Bis-boc-guanidino-butylamine)-guinoline-3-carboxylic acid ethyl ester (B2)

To a solution of 1,3-Di-boc-2-(trifluoromethylsulfonyl)guanidine (391 mg, 1 mmol) in dry DCM (4 mL) was added ethyl 4-(4-aminobutylamino)quinoline-3-carboxylate (A3) (0.3 g, 1.05 mmol) neat and the mixture stirred at room temperature for 15 h. The mixture was diluted with DCM and washed with 2M NaHSO₄ (20 mL), Sat. NaHCO₃ (20 mL), Sat. NaCl (20 mL), dried (MgSO₄), filtered and concentrated to give the product as a white foam (225 mg) that was used as is in the subsequent step.

4-(4-Ruanidino-butylamine)-guinoline-3-carboxylic acid (B3)

To a solution of 4-(4-Bis-boc-guanidino-butilamine)-quinoline-3-carboxylic acid ethyl ester (B2) (255 mg, 0.43 mmol) in DME (2 mL) was added 1M NaOH (2 mL) and the solution stirred at room temperature for 6 h. To this solution was added 2 drops of 20% KOH solution and stirring continued for 15 h. The solution was concentrated to ⅓ the volume and the pH was adjusted to pH-6 with IM HCl and the resulting white ppt collected by filtration and dried to give the product as a white solid. (0.132 g, 0.26 mmol, 61%)

To a solution of the white solid (152 mg, 0.27 mmol) in DCM (2mL) was added TFA (2 mL) and the mixture stirred at room temperature for 2 h. The mixture was concentrated under reduced pressure and the resulting residue was purified by reverse phase HPLC, H₂O/ACN/0.1%TFA (5% -95%ACN) and the resulting fractions concentrated by lyophillization to give the product as a white solid (43 mg, 0.1 mmol, 34%). MP-98° C., ¹H NMR (400MHz) δ8.82 (s, 1H), 8.49 (d, J=8.4Hz, 1H), 8.08 (s, 1H), 7.86 (m, 2H), 7.56 (t, J=7.6, 7.2Hz, 4H), 7.31 (br s, 4H), 3.98 (s, 2H), 3.20 (d, J=5.6Hz, 3H), 1.75 (dd, J=6.4,36.4Hz, EIMS m/z M⁺¹ 302.3. Anal. C₁₅H₁₉N₅O₂+1 TFA+2 H₂O 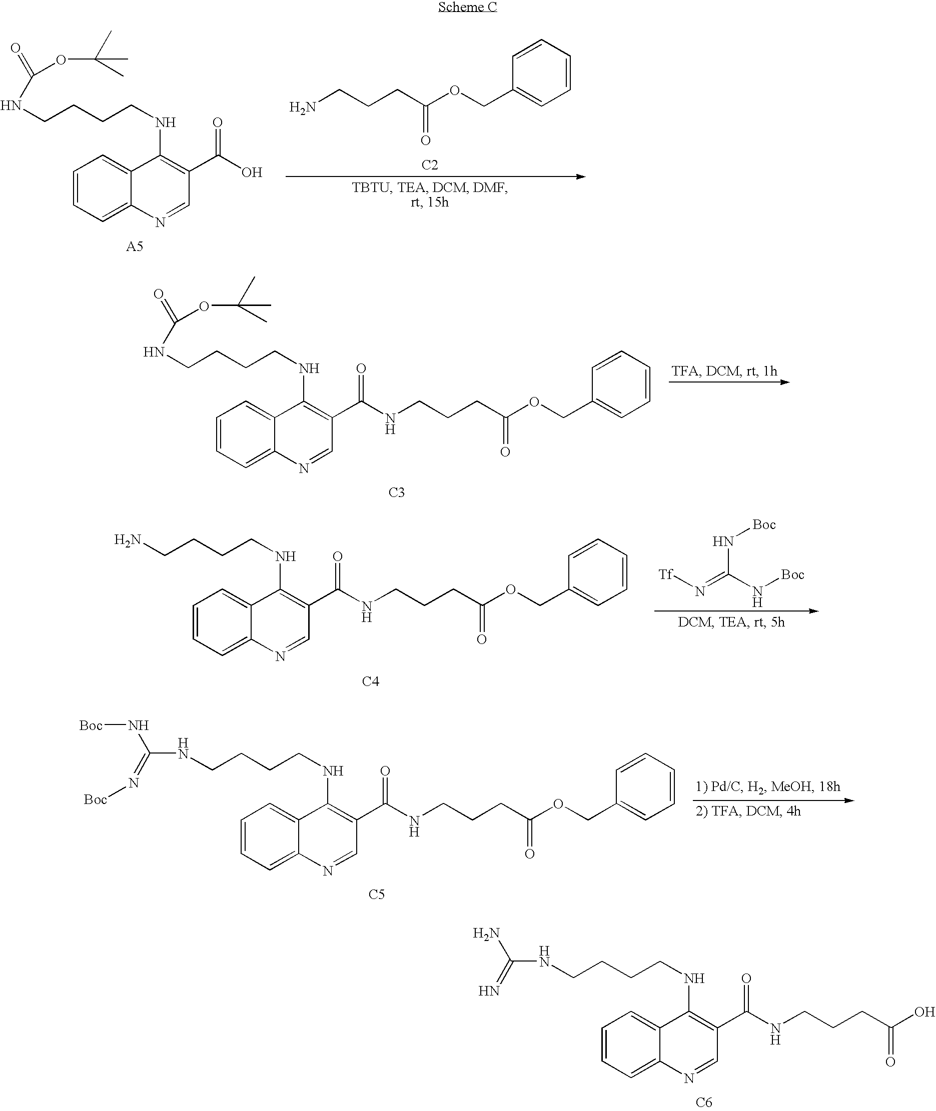
Scheme C
4-{[4-(4-tert-Butoxycarbonylamino-butylamino)-guinoline-3-carbonyl]-amino}-butyric acid benzyl ester (C3)

To a suspension of 4-(4-tert-Butoxycarbonylamine-butylamino)-qunoline-3-carboxylic acid (A5) (0.5 g, 1.4 mmol) in DCM (20 mL) was added TBTU (1.1 eq, 1.53 mmol, 482 mg) and the solution stirred and DMF (20 mL) was added after 8 h. After 28 h of continuous stirring the solution went clear and TEA (155 mg, 0.213 mL, 1.53 mmol) was added followed by benzyl 4-aminobutanoate (C2) (1.1 eq, 0.559 g,1.53 mmol) and the mixture stirred for 15 h. The DCM was removed under reduced pressure and the remainder diluted with water. This aqueous solution was extracted with ether (3×50 mL) and then DCM (3×50 mL). The organic layers were combined, dried (MgSO₄), filtered, and concentrated. The residue was purified by flash chromatography over silica using DCM/MeOH (9:1) to give the product as an oil (0.484 g) of sufficient purity for use in following steps.

4-{[4-(4-Amino-butylamino)-guinoline-3-carbonyl]-amino}-butyric acid benzyl ester (C4)

To a solution of 4{[4-(4-tert-Butoxycarbonylamino-butylamino)-quinoline-3-carbonyl]-amino}-butyric acid benzyl ester (C3) (0.454 mg, 0.9 mmol) in DCM (10 mL) was added TFA (4 mL) and the mixture was stirred for 1 h. The solution was concentrated, neutralized with saturated NaHCO₃, and extracted with DCM. The organic layers were combined, dried (MgSO₄), filtered, and concentrated under reduced pressure to give the product as a clear oil (227 mg, 0.52 mmol).

4-{[4-(4-bis-Boc-guanidino-butylamino)-guinoline-3-carbonyl]-amino}-butyric acid benzyl ester (C5)

To a solution of 4{[4-(4-Amino-butylamino)-quinoline-3-carbonyl]-amino}-butyric acid benzyl ester (C4) (227 mg, 0.52 mmol) in DCM (7 mL) was added TEA (53 mg, 0.72 mL) followed by 1,3-Di-boc-2-(trifluoromethylsulfonyl)guanidine (204 mg, 0.52 mmol) and the mixture stirred at room temperature for 5 h. The organic solution was diluted with more DCM washed with 2M NaHSO₄ (25 mL), NaHCO₃ (25 mL), dried (MgSO₄), filtered and concentrated under reduced pressure to give the product as a white foam (305 mg) that was used as is.

4-{[4-(4-Guanidino-butylamino)-quinolin-3-carbonyl]-amino}-butyric acid (C6)

To a solution of 4-{[4-(4-bis-Boc-guanidino-butylamino)-quinoline-3-carbonyl]-amino}-butyric acid benzyl ester (C5) (305 mg) in MeOH (10 mL) was added Pd/C (10 wt%, 10 %wt/wt, 30 mg) and the mixture vacuum purged 5× with H₂ gas and stirred under H₂ for 18 h. The Pd/C was removed by filtration through celite and the filtrate concentrated under reduced pressure to give a white foam residue.

The above residue was dissolved in DCM (5 mL) and TFA (5 mL) was added and the mixture stirred at room temperature for 4 h. The solvents were removed under reduced pressure and the residue triturated with ether. The resulting oil was purified by reverse phase HPLC using ACN/H₂O/TFA (0.1%) as eluent (gradient from 5%-95% ACN) to give the product as a white hygroscopic solid (70 mg, 0.018 mmol). MP-not determined, ¹H NMR (400 MHz) δ9.86 (br s, 1H), 8.92 (t, J=5.2, 5.6Hz, 1H), 8.56 (d, J=8.8Hz, 1H), 7.91 (m, 2H), 7.76 (t, J=5.6, 5.6Hz, 1H), 7.68 (m, 1H), 7.37-7.06 (br m, 4H), 3.29 (m, 6H), 3.12 (q, J=6.4, 12.8Hz), 2.33 (t, J=7.2, 7.2Hz, 2H), 1.76 (m, 4H), 1.54 (m, 2). EIMS m/z M⁺¹ 387.5. Anal. Not determined. 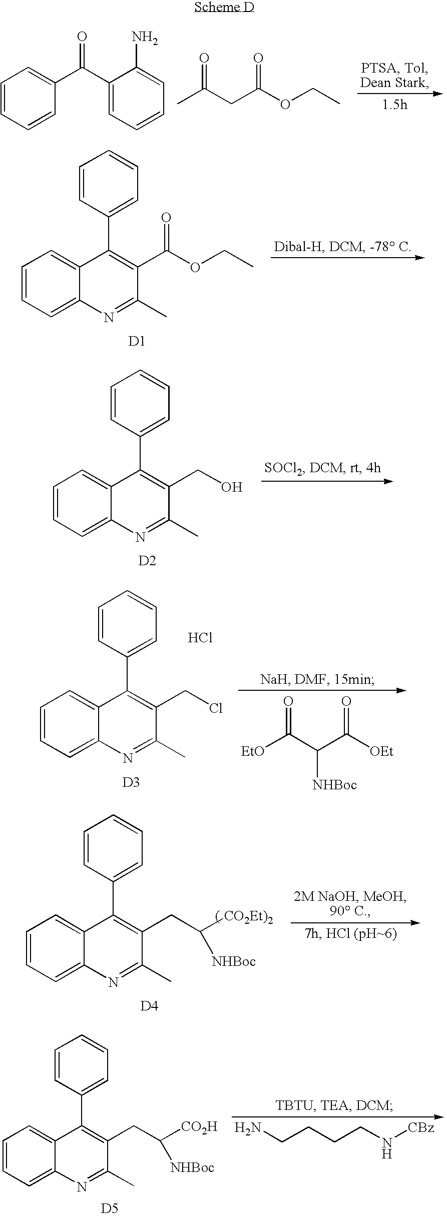 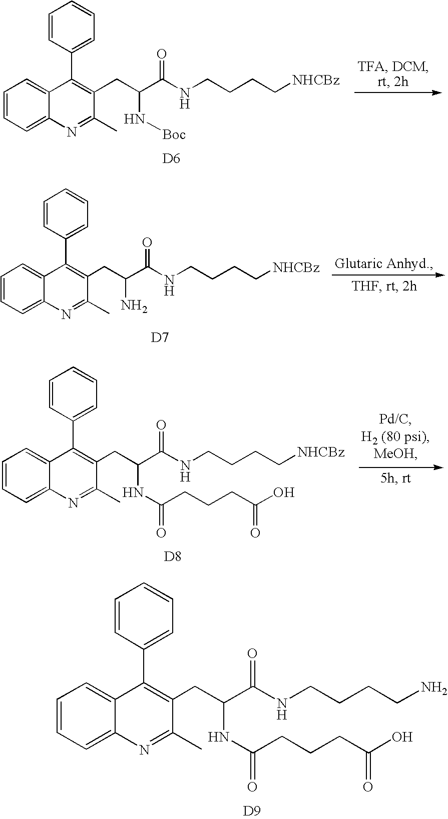
Scheme D
Ethyl 2-methyl-4-phenylquinolin-3-carboxylate (D1)

To a solution of 2-aminobenzophenone (10 g, 51 mmol) and ethylacetoacetate (5.3 g, 63.8 mmol, 8 mL) in toluene (100 mL) was added PTSA (0.3 g) and the reaction mixture heated at reflux using a Dean Stark apparatus for 1.5 h when no more water was apparent. The solvent was removed under reduced pressure and the residue recrystallized from EtOH to give the product as light yellow crystals. (8.14 g, 28 mmol)

(2-Methyl-4-phenylguinolin-3-yl)methanol (D2)

To a solution of ethyl 2-methyl-4-phenylquinolin-3-carboxylate (DI) (5 g, 17.2 mmol) in DCM (50 mL) at −78° C. was added 1M Dibal-H (2.5eq, 43 mmol, 43 mL) in DCM dropwise and stirring continued at this temperature for 1.5 h. A solution of Na₂SO₄ (6.1 g, 43 mmol) in water (10 mL) was added carefully at −78° C. and the mixture allowed to warm to room temperature and stirred for 1 h. The solid was filtered off and rinsed with hot EtOAc. The filtrates were combined and concentrated under reduced pressure to give the product as a yellow solid residue (3.62 g, 14.5 mmol)

3-(Chloromethyl)-2-methyl-4-phenylguinoline (D3)

To a solution of (2-Methyl-4-phenylquinolin-3-yl)methanol (D2) in DCM (50 mL) was added SOCl₂ (10.4 mL, 17 g, 140 mmol) and the mixture stirred at room temperature for 4 h. The mixture was concentrated under reduced pressure to give the HCl salt of the chloride as a yellow solid (2.266 g. The product was stored as the HCl salt and converted to the free base by treating with saturated NaHCO₃ and extracting with ether.

tert-Butyl 1,1-di(ethoxycarbonyl)-2-(2-methyl-4-phenylguinolin-3-yl)ethylcarbamate (D4)

To a solution of 3-(Chloromethyl)-2-methyl-4-phenylquinoline (D3) (0.958 g, 3.6 mmol) as the free base in DMF (12 mL) was added a DMF (40 mL) solution of tert-butyl di(ethoxycarbonyl)methylcarbamate (4.32 mmol, 1.19 g) that had been deprotonated by treating with NaH (4.32 mmol, 104 mg) for 15min. This mixture was stirred overnight and then concentrated under reduced pressure, dissolved in H₂O and the solution extracted with ether (3×50 mL). The extracts were combined, dried (MgSO₄), filtered, concentrated under reduced pressure to give the product as a brown oil that was used as is.

2-tert-Butoxycarbonylamino-3-(2-methyl-4-phenyl-quinolin-3-yl)-propionic acid (D5)

To a solution of tert-butyl 1,1 -di(ethoxycarbonyl)-2-(2-methyl-4-phenylquinolin-3-yl)ethylcarbamate (D4) (0.85 g, 1.7 mmol) in MeOH (10 mL) was added 2M NaOH (2.1 eq, 1.8 mL) and the mixture heated at 90° C. for 7 h. The solvent was removed under reduced pressure and the residue diluted with water. The resulting mixture was adjusted to pH-5.5 using 20% aqueous HCl and the milky white solution extracted with EtOAc (3×50 mL), dried (MgSO₄), filtered, and concentrated to give the product as a brown foam solid (0.404 g, 1 mmol) that was used as is.

(3 -[2-tert-Butoxycarbonylamin-3-(2-methyl-4-phenyl-quinolin-3 -yl)-propionylamino]-propyl}-carbamic acid phenyl ester (D6)

To a solution of 2-tert-Butoxycarbonylamino-3-(2-methyl-4-phenyl-quinolin-3-yl)-propionic acid (D5) (200 mg, 0.5 mmol) in DCM (15 mL) was added TBTU (1.1 eq, 174 mg, 0.54 mmol) and TEA (2eq, 1.08 mmol, 110 mg, 150 μL) and the mixture allowed to stir at room temperature for 10min. To this mixture was added phenyl 4-aminobutylcarbamate (1.1 eq, 0.54 mmol, 140 mg) and the mixture stirred at room temperature for 4 h. Water was added and the organic layer separated, dried (MgSO₄), filtered, and concentrated to give a yellow residue. The residue was purified over silica using first 2%DCM/MeOH, then 4%DCM/MeOH, then 8%DCM/MeOH, 250 mL ea. to give the product as a yellow oil (228 mg).

{3-[-Amino-3-(2-methyl-4-phenyl-quinolin-3-yl)-proionylamino]-butyl}-carbamic acid phenyl ester (D7).

To a solution of {3-[2-tert-Butoxycarbonylamin-3-(2-methyl-4-phenyl-quinolin-3-yl)-propionylamino]-propyl}-carbamic acid phenyl ester (D6) (220 mg, 0.36 mmol) in DCM (5 mL) and a mixture of TFA/DCM (3.5 mL/5 mL) was added and the mixture stirred at room temperature for 2 h. The solvent was removed under reduced pressure and the residue taken up in EtOAc (50 mL) and washed with saturated NaHCO₃ (35 mL), water (2×25 mL), dried (MgSO₄), filtered, and concentrated to give the product as a clear yellow oil (161 mg, 0.32 mmol, 88%).

4-[2-(2-Methyl-4-phenyl-quinolin-3-yl)-1-(4-phenoxycarbonyl amino-butyl carbamoyl)-ethyl carbamoyl]-butyric acid (D8)

To a solution of {3-[-Amino-3-(2-methyl-4-phenyl-quinolin-3-yl)-proionylamino]-butyl}-carbamic acid phenyl ester (D7) (161 mg, 0.32 mmol) in THF was added glutaric anhydride (1.5 eq, 0.5 mmol, 57 mg) and the solution stirred at room temperature for 2 h. The solvent was removed under reduced pressure and the residue taken up in EtOAc (25 mL) and washed with water, dried (MgSO₄), filtered, and concentrated to give the product as a clear orange oil that slowly solidified (213 mg) that was used as is without further characterization.

4-[1-(4-Amino-butylcarbamoyl)-2-(2-methyl-4-phenyl-quinolin-3-yl)-ethyl carbamoyl]-butyric acid (D9)

To a solution of 4-[2-(2-Methyl-4-phenyl-quinolin-3-yl)-1-(4-phenoxycarbonyl amino-butyl carbamoyl)-ethyl carbamoyl]-butyric acid (D8) (213 mg, 0.34 mmol) in MeOH (10 mL) and THF (5 mL) was added Pd/C and placed on a Parr shaker at 80 psi H₂ gas for 5 h. The Pd/C was removed by filtration through celite and concentrated. The resulting residue was purified using reverse phase HPLC using H₂O/ACN (5-95%ACN) giving the product as a white solid after lyophillization (13.3 mg). MP 129° C., ¹H NMR (400 MHz) δ7.91 (m, 2H), 7.64 (m, 2H), 7.54 (m, 4H), 7.36 (m, 2H), 7.26 (d, J=6.4Hz, 1H), 7.08 (d, J=8Hz, 1H), 4.352 (m, 1H), 3.1-2.6 (series of m, 8H), 2.03 (m, 4H), 1.55 (m, 2H), 1.22 (m, 4H). EIMS m/z M⁺¹ 491.7. Anal. C₂₈H₃₄N₄O₄+3H₂O 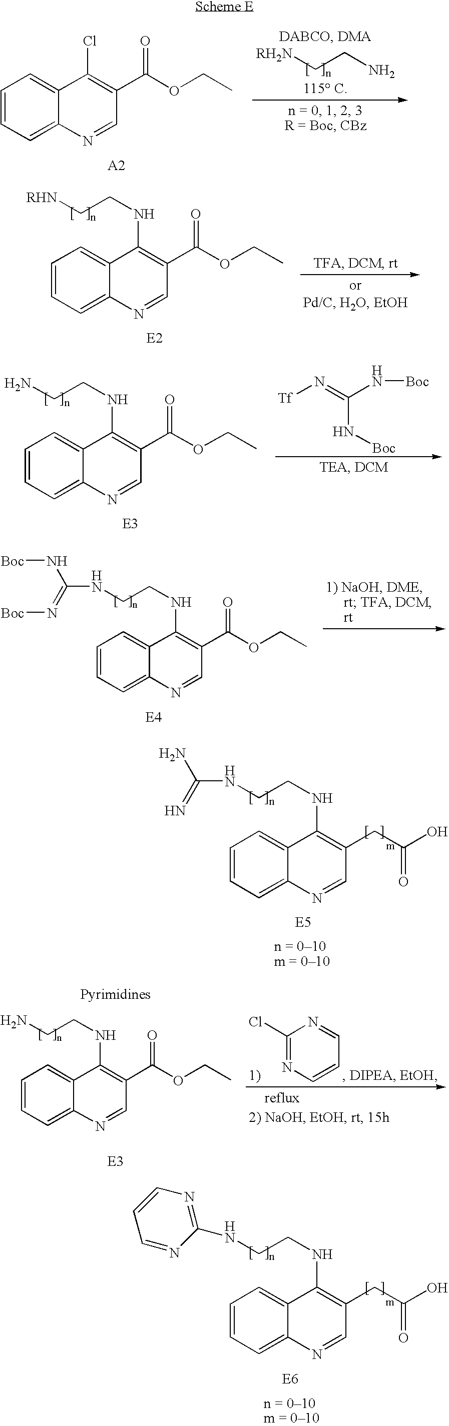 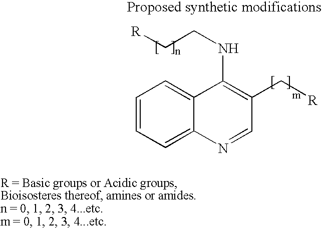
Scheme E
4-(5-Benzyloxycarbonylamino-pentylamino)-guinoline-3-carboxylic acid ethyl ester (E2, n=4)

To a solution of ethyl 4-chloroquinoline-3-carboxylate (A2) (1 g, 4.26 mmol) in DMA (20 mL) was added N-CBz-diaminopentane (1.4 g, 5.1 mmol) and DABCO (1.4 g, 13 mmol) and the solution heated at 115° C. for 2.5 h. The DMA was removed under reduced pressure and the residue suspended in water and extracted with ether (3×25 mL), dried (MgSO₄), filtered, and concentrated to give the product as a clear brown oil (1.88 g, 4.3 mmol) that was used as is. 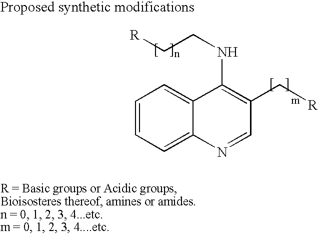
4-(5-Amino-pentylamino)-quinoline-3-carboxylic acid ethyl ester (E3. n=4)

To a solution of 4-(5-Benzyloxycarbonylamino-pentylamino)-quinoline-3-carboxylic acid ethyl ester (E2, n=4) (1.88 g, 4.3 mmol) in EtOH (30 mL) was added Pd/C (180 mg, 10%ww Pd) and the mixture stirred under H₂ gas for 3d refilling the balloon as necessary. The catalyst was removed by filtering through celite and concentrated to give the product as a honey colored oil (1.3 g, 4.2 mmol) that was used without further purification.

4-(5-Bis-Boc-guanidino-pentylamino)-guinoline-3-carboxylic acid ethyl ester (E4, n=4)

To a solution 4-(5-Amino-pentylamino)-quinoline-3-carboxylic acid ethyl ester (E3, n=4) (0.64 g, 2.1 mmol) in dry DCM (10 mL) was added TEA (322 ul, 233 mg) and 1,3-Di-boc-2-(trifluoromethylsulfonyl)guanidine (1.1 eq, 0.9 g, 2.31 mmol) and the mixture stirred at room temperature for 2.5 h. The solution was diluted with more DCM and washed with 2M NaHSO₃ (20 mL), saturated NaHCO₃ (20 mL), saturated NaCl, dried (Na₂SO₄), filtered and concentrated to give the product as white foam (1.2 g, 2.1 mmol) that was used as is.

4-(5-Guanidino-pentylamino)-guinoline-3-carboxylic acid (E5 n=4)

To a solution of 4-(5-Bis-Boc-guanidino-pentylamino)-quinoline-3-carboxylic acid ethyl ester (E4, n=4) (1.2 g, 2.1 mmol) in DME (20 mL) was added 1M NaOH (15 mL) and the mixture stirred at room temperature for 2d. The mixture was concentrated to remove the DME and the remaining aqueous mixture adjusted to pH˜5-6 with HCl (20% aqueous). The resulting solid was collected by filtration and air dried.

The crude solid was suspended in DCM (15 mL) and TFA (3.5 mL) was added and the mixture at room temperature for 2.5 h. More TFA was added and the solution stirred for 3.5 h and then concentrated. The residue was suspended in water and 2M Na₂CO₃ added to adjust to pH-7-8 and the resulting solid collected by filtration and dried in under vacuum. The crude was purified by reverse phase HPLC over C18 using H₂O/ACN/0.5%TFA to give the compound as a white solid after lyophillization (40 mg, 0.09 mmol) as the mono TFA salt. MP 72° C., ¹H NMR (400 MHz) δ8.79 (s, 1H), 8.48 (d, J=8.8Hz, 1H), 7.86 (m, 2H), 7.78 (m, 1H), 7.55 (m, 1H), 7.24 (br s, 4H), 3.94 (m, 3H), 3.14 (d, J=6.4, 6.8Hz, 2H), 1.77 (m, 2H), 1.55 (m, 4H) EIMS m/z M⁺¹ 316.3. Anal. C₁₆H₂₁N₅O₂+2H₂O+1 TFA

4-(3-Guanidino-propylamino)-guinoline-3-carboxylic acid (E5, n=2)

This compound was made in a manner similar to 4-(5-Guanidino-pentylamino)-quinoline-3-carboxylic acid (E5, n=4) using n-(3-aminopropyl)-carbamic acid t-butyl ester and deprotecting with TFA. MP 231° C., ¹H NMR (400 MHz) δ10.48 (m, 1H), 9.52(br s, 1H), 9.00 (s, 1H), 8.20 (d, J=8.4Hz, 1H), 7.75 (d, J=8.4Hz, 1H), 7.60 (t, J=6.8,8.4Hz, 2H), 7.35 (t, J=7.6, 8Hz, 2H), 3.75 (m, 3H), 3.22 (t, J=7.2, 7.2Hz, 2H), 1.90 (m, 2H) EIMS m/z M⁺¹ 288.4. Anal. C₁₄H₁₇N₅O₂+2H₂O

4-(2-Guanidino-ethylamino)-quinoline-3-carboxylic acid (E5, n=1)

This compound was made in a manner similar to 4-(5-Guanidino-pentylamino)-quinoline-3-carboxylic acid (E5, n=4) using n-Boc-ethylene diamine and deprotecting with TFA. MP 267° C., ¹H NMR (400 MHz) δ8.77 (s, 1H), 8.42 (d, J=8.4Hz, 1H), 7.84 (m, 3H), 7.57 (t, J=8.4Hz, 7.2Hz 1H), 7.28 (br s, 3H), 4.08 (br s, 2H). EIMS m/z M⁺¹ 274.5. Anal. C₁₃H₁₅N₅O₂+2H₂O+1 TFA

Pyrimidines

4-[3-(Pyrimidin-2-yl-amino)-propylamino]-quinoline-3-carboxylic acid (E6, n=2, R=H)

To a solution of 4-(3-Amino-propylamino)-quinoline-3-carboxylic acid ethyl ester (E3, n=2, 177 mg, 0.65 mmol) in EtOH (35 mL) was added DIPEA (1 mmol, 129 mg, 173 uL) and 2-chloropyrimidine (90 mg, 0.78 mmol) and the mixture heated at reflux for 15 h. The solution was concentrated and taken up in EtOH (15 mL) and 1M NaOH (5 mL) added and the solution stirred for 15 h. The mixture was concentrated and the residue adjusted to pH˜5 using 20% HCl. The resulting solid was collected and purified on reverse phase HPLC, ACN/H₂O 5-95% on C18 to give the product as a white solid after lyophillization (135 mg). MP 269° C. ¹H NMR (400 MHz) δ8.47 (d, J=8.8Hz, 1H), 8.19 (d, J=4.8Hz, 2H), 7.80 (m, 2H), 7.50 (m, 1H), 7.26(m, 1H), 6.52 (t, J=4.4, 5.2Hz, 1H), 3.99 (m, 2H) 2.00 (m, 2H). EIMS m/z M⁺¹ 324.5. Anal. C₁₇H₁₇N₅O₂+1H₂O+1 TFA

4-[5-(Pyrimidin-2-ylamino)-pentylamino]-quinoline-3-carboxylic acid ethyl ester (E6, n=4, R═CH₂CH₃)

To a solution of 4-(5-Amino-pentylamino)-quinoline-3-carboxylic acid ethyl ester (E3, n=4, 505 mg, 1.7 mmol) in EtOH (20 mL) was added DIPEA (323 mg, 2.5 mmol, 435 uL) and 2-chloropyrimidine (231 mg, 2 mmol) and the mixture heated at reflux for 1 5 h. The mixture was concentrated and the residue purified by reverse phase HPLC, C 18, ACN/H₂O, 5-95% to give the product as an off white yellowish solid (135 mg, 0.36 mmol, 21%). MP 108° C. ¹H NMR (400 MHz) δ8.92 (m, 1H), 8.83 (s, 1H), 8.34 (d, J=8.4Hz, 1H), 8.19 (d, J=4.8Hz, 2H), 7.8 (m, 1H), 7.71 (m, 1H), 7.44 (m, 1H), 7.09 (t, J=6, 5.6Hz, 1H), 6.50 (t, J=4.8, 4.8Hz, 1H), 3.68 (m, 2H), 3.22 (q, J=6.8, 12.8Hz, 2H), 1.68 (m, 2H), 1.52 (m, 2H), 1.41 (m, 2H). EIMS m/z M⁺¹ 380.5. Anal. C₂₁H₂₅N₅O₂ 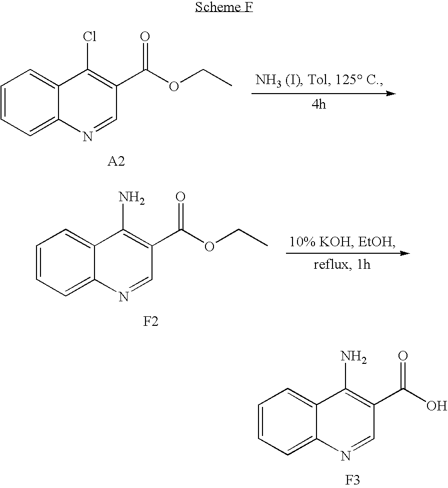
Scheme F
4-Amino-quinoline-3-carboxylic acid ethyl ester (F2)

To a solution of ethyl-4-chloro quinoline-3-carboxylate (A2, 1.44 g, 0.6 mmol) in toluene (10 ML) was added condensed NH₃ and the mixture sealed in a steel bomb and heated at 125° C. for 4 h. The bomb was cooled and the resulting white solid was collected by vacuum filtration and dried to give the product (1.5 g).

4-Amino-quinoline-3-carboxylic acid (F3)

To a solution of 4-Amino-quinoline-3-carboxylic acid ethyl ester (F2) (250 mg, 1.2 mmol) in EtOH (5 mL) was added 20% KOH (10 mL) and the mixture heated at reflux for 1 h. The EtOH was removed under reduced pressure and the aqueous solution adjusted to pH˜6.5-7 using 20%HCl. The white solid was collected and dried to give the product. (161 mg). The product was crystallized from EtOH and dried. MP 305° C. ¹H NMR (400 MHz) δ8.89 (s, 1H), 8.42 (d, J=8.4Hz, 1H), 7.83 (m, 2H), 7.60 (m, 1H). EIMS m/z M⁺¹ 189.4. Anal. C₁₀H₈N₂O₂+0.5 H₂O

Many modifications and variations of the embodiments described herein may be made without parting from the scope, as is apparent to those skilled in the art. The specific embodiments described herein are offered by way of example only.

### REFERENCES CITED (And Incorporated Herein by Reference Thereto)

Alam et al. (2001) J. Biol. Chem. 276, 15641-15649
Anantharamaiah et al. (1985) J. Biol. Chem. 260, 10248-10255
Anantharamaiah et al. (1987) J. Lipid Res. 29, 309-318
Anantharamaiah et al. (1990) Arteriosclerosis, 10, 95-105
Arai et al. (1999) J. Biol. Chem. 274, 2366-2371
Argraves et al. (1997) J. Clin. Invest. 100, 2170-2181
Austin et al. (1988) JAMA, 260, 1917-1921
Austin et al. (1990) Circulation, 82, 495-506
Banka et al. (1994) J. Biol. Chem. 269, 10288-10297
Barbaras et al. (1987) Biochem. Biophys. Res. Commun. 142, 63-69
Barter P (2000) Arterioscl. Thromb. Vasc. Biol. 20, 2029
Bemeis and Krauss (2002) J. Lipid Res. 43, 1363-1379
Bhatnagar A. (1999) in Lipoproteins and Health Disease, pp. 737-752, Arnold, Loudon
Bolibar et al. (2000) Thromb. Haemost. 84, 955-961
Boren et al. (2001) J. Biol. Chem. 276, 9214-9218
Brouillette and Anantharamaiah (1995) Biochim. Biophys. Acta. 1256, 103-129
Brunzell J D (1995) in The Meatbolic and Molecular Bases of Inherited Disorders, pp. 1913-1932, McGraw-Hill, Inc., New York
Buchko et al. (1996) J. Biol. Chem. 271, 3039-3045
Camejo et al. (1985) Atherosclerosis, 55, 93-105
Campos et al. (1992) Arteriosclerosis Thrombosis, 12, 187-193
Canner et al. (1986) JACC, 8, 1245-1255
Cao et al. (2002) J. Biol. Chem. 277, 39561-39565
Castelli et al. (1986) JAMA, 256, 2835-2838
Castro and Fielding (1998) Biochemistry, 27, 25-29
Chait et al. (1993) Am. J Med. 94, 350-356
Chambenoit et al. (2001) J. Biol. Chem. 276, 9955-9960;
Chang et al. (1997) Annu Rev Biochem. 66, 613-638
Chapman et al. (1998) Eur Heart J, Suppl A: A24-30
Chen and Albers (1985) Biochim Biophys. Acta, 836, 275-285
Chen et al. (2000) J. Biol. Chem. 275, 30794-30800
Cohen et al. (1999) Curr Opin Lipidol. 10, 259-268
Collet et al. (1997) J. Lipid Res. 38, 634-644
Collet et al. (1999) J. Lipid Res. 40, 1185-1193
Curtiss and Boisvert (2000) Curr. Opin. Lipidol. 11, 243-251
Datta et al. (2001) J. Lipid Res. 42, 1096-1104
Davis et al. (2002) J. Lipid Res. 43, 533-543
de Graaf et al. (1993) J. Clin. Endocrinol. Metab. 76, 197-202
Downs et al. (1998) JAMA, 279, 1615-1622
Duverger et al. (1996) Circulation, 94, 713-717
Ehnholm et al. (1998) J. Lipid Res. 39, 1248-1253
Epand et al. (1987) J. Biol. Chem. 262, 9389-9396
Eriksson et al. (1999) Circulation, 100, 594-598
Fan et al. (2001) J. Biol. Chem. 276, 40071-40079
Fidge N H (1999) J. Lipid Res. 40, 187-201
Fielding et al. (1994) Biochemistry, 33, 6981-6985
Fitch W M (1977) Genetics, 86, 623-644
Fogelman et al. (2003) United States Patent Application Publication, US 2003/0045460 A1
Föger et al. (1996) Arterioscler Thromb Vasc Biology, 16, 1430-1436
Foger et al. (1999) J. Biol. Chem. 274, 36912-36920
Frank and Marcel (2000) J. Lipid Res. 41, 853-872
Frick et al. (1987) N. England J. Medicine, 317, 1237-1245
Fuskushima et al. (1980) J. Biol. Chem. 255, 10651-10657
Gamble et al. (1978) J. Lipid Res. 16,1068-1070
Garber et al. (1992) Arteriosclerosis and Thrombosis, 12, 886-894
Garber et al. (2001) J. Lipid Res. 42, 545-552
Garcia et al. (1996) Biochemistry, 35, 13064-13071
Genestetal. (1991) Am J Cardiol. 67, 1185-1189
Genest et al. (1992) Circulation, 85, 2025-2033
Genest et al. (1999) J. Invest. Med. 47, 31-42
Gibbons et al. (1995) Am. J. Med. 99, 378-385
Gillotte et al. (1999) J. Biol. Chem. 274, 2021-2028
Glomset J A (1968) J. Lipid Res. 9, 155-167
Goldberg I. (1996) J. Lipid Res. 37, 693-707
Golder-Novoselsky et al. (1995) Biochim. Biophys. Acta, 1254, 217-220
Goldstein and Brown (1974) J. Biol. Chem. 249,5153-5162
Gordon et al. (1989) N Engl. J. Med. 321, 1311-1315
Gotto A M (2001) Circulation, 103, 2213
Griffin et al. (1994) Atherosclerosis, 106, 241-253
Groen et al. (2001) J. Clin. Invest. 108, 843-850
Hajjar and Haberland (1997) J. Biol. Chem. 272, 22975-22978
Hara and Yokoyama (1991) J. Biol. Chem. 266, 3080-3086
Hedrick et al. (2001) J. Lipid Res. 42, 563-570
Huang et al. (1995) Arterioscler. Thromb. Vasc. Biology, 15, 1412-1418
Huang et al. (1997) Arterioscler. Thromb. Vasc. Biol. 17, 2010-2015
Hulley et al. (1998) JAMA, 280, 605-613
Huuskonen and Ehnholm (2000) Curr. Opin. Lipidol. 11, 285-289
Huuskonen et al. (2000) Atherosclerosis, 151, 451-461
Ikewaki et al. (1993) J. Clin. Invest. 92, 1650-1658
Ikewaki et al. (1995) Arterioscler. Thromb. Vasc. Biology, 15, 306-312
Ishigami et al. (1994) J. Biochem. (Tokyo) 116, 257-262
Jaakkola et al. (1993) Coron. Artery Dis. 4, 379-385
Jauhianen et al. (1993) J. Biol. Chem. 268, 4032-4036
Jiang et al. (1996) J. Clin. Invest. 98, 2373-2380
Jiang et al. (1999) J. Clin. Invest. 103, 907-914
Jonas A (1991) Biochim. Biophys, Acta, 1084, 205-220
Jones et al. (1998) Am. J. Cardiol. 81, 582-587
Kaiser and K\check{e}zdy (1983) Proc. Natl. Acad. Sci. USA, 80, 1137-1140
Kanellis et al. (1980) J. Biol. Chem. 255, 11464-11472
Kawano et al. (2000) J. Biol. Chem. 275, 29477-29481
Kozarsky et al. (2000) Arterioscler. Thromb. Vasc. Biology, 20, 721-727
Krauss and Burke (1981) J. Lipid Res. 23, 97-104
Krieger M. (1998) Proc. Natl. Acad. Sci. USA. 95, 4077-4080
La Belle and Krauss (1990) J. Lipid Res.,31, 1577-1588
Lindholm et al. (1998) Biochemistry, 37, 4863-4868
Liu and Krieger (2002) J. Biol. Chem. 277, 34125-34135
Lund-Katz et al. (1993) In “Peptides: Chemistry and Biology” (R. Haughten, ed.) ESCOM Press, Leiden, The Netherlands
Lusa et al. (1996) Biochem. J. 313, 275-282
Main et al. (1996) Biochim Biophys Acta, 29, 17-24
Marotti et al. (1993) Nature, 364, 73-75
Martin-Jadraque et al. (1996) Arch. Intern. Med. 156, 1081-1088
Marzal-Casacuberta et al. (1996) J. Biol. Chem. 271, 6720-6728
Matsumoto et al. (1997) J. Biol. Chem. 272, 16778-16782
McLachlan A D (1977) Nature, 267, 465-466
McLean et al. (1991) Biochemistry, 30, 31-37
McManus et al. (2000) J. Biol. Chem. 275, 5043-5051
Mendez et al. (1994) J. Clin. Invest. 94, 1698-1705
Meng et al. (1995) J. Biol. Chem. 270, 8588-8596
Merkel et al. (2002) J. Lipid Res. 43, 1997-2006
Miccoli et al. (1997) J. Lipid Res. 38, 1242-1253
Miettinen et al. (1997) Arterioscler. Thromb. Vasc. Biology, 17, 3021-3032
Miller et al. (1987) Am Heart J. 113, 589-597
Milner et al. (1991) Biochim Biophys Acta, 26, 1082, 71-78
Mishra et al. (1994) J. Biol. Chem. 269, 7185-7191
Mishra et al. (1995) J. Biol. Chem. 270, 1602-1611
Mishra et al. (1998) Biochemistry, 37, 10313-10324
Morton R E (1999) Curr Opin Lipidol. 10, 321-327
Nagano et al. (2002) J. Lipid Res. 43, 1011-1018
Naito H K (1985) Ann. NY Acad. Sci, 454, 230-238
Nakagawa et al. (1985) J. Am. Chem. Soc. 107, 7087-7092
Ohnishi and Yokoyama (1993) Biochemistry, 32 (19), 5029-5035
Oka et al. (2000) Clin. Chem. 46, 1357-1364
Oka et al. (2000) J. Lipid Res. 41, 1651-1657
Oka et al. (2002) J. Lipid Res. 43, 1236-1243
Okarnoto et al. (2000) Nature, 13, 406 (6792): 203-7
Oram and Lawn (2001) J. Lipid Res. 42, 1173-1179
Oram and Yokoyama (1997) J. Lipid Res. 37, 2473-2491
Packard and Shepherd (1997) Arteriosclerosis, Thromb, Vasc. Biology, 17, 3542-3556
Palgunachari et al. (1996) Arterioscler. Thromb. Vasc. Biol. 16, 328-338
Plump et al. (1997) Prc. Natl. Acad. Sci. USA, 91, 9607-9611
Ponsin et al. (1986a) J. Biol. Chem. 261, 9202-9205
Ponsin et al. (1986b) J. Clin. Invest. 77, 559-567
Pownall et al. (1980) Proc. Natl. Acad. Sci. USA, 77(6), 3154-3158
Pownall et al. (1985) Biochim. Biophys..Acta, 833, 456-462
Puchois et al. (1987) Atherosclerosis, 68, 35-40
Pussinen et al. (1997) J. Lipid Res. 38, 12-21
Pussinen et al. (1998) J. Lipid Res. 39, 152-161
Qin et al. (2000) J. Lipid Res. 41, 269-276
Ramsamy et al. (2000) J. Biol. Chem. 275, 33480-33486
Remaley et al. (1997) Arterioscler Thromb. Vasc. Biology, 17, 1813-1821
Reschly et al. (2002) J. Biol. Chem. 277, 9645-9654
Riemens et al. (1998) Atherosclerosis, 140, 71-79
Riemens et al. (1999) J. Lipid Res. 40, 1459-1466
Rinninger et al. (1998) J. Lipid Res. 39, 1335-1348
Ross R. (1993) Nature, 362, 801-809
Rothblat et al. (I999) J. Lipid Res. 40, 781-796
Rubin et al. (1991) Nature, 353, 265-267
Rubins, et al. (1999) N. Engl. J. Med. 341, 410-418
Santamarina-Fojo and Dugi (1994) Curr. Opin. Lipidol. 5, 117-125
Santamarina-Fojo et al. (2000) Curr. Opin. Lipidol. 11, 267-275
Schissel et al. (1996) J. Clin. Invest. 98, 1455-1464
Second Report of the Expert Panel (1994) Circulation, 89, 1329-1445
Segrest et al. (1983) Journal Biol Chem. 258, 2290-2295
Segrest et al. (1994) Advances in Protein Chem. 45, 303-369
Segrest et al. (2001) J. Lipid Res. 42, 1346-1367
Segrest J P (1974) FEBS Lett. 38, 247-253
Settasation et al. (2000) J. Biol. Chem. 276, 26898-26905
Shaefer E J (1994) Eur. J. Clin. Invest. 24, 441-443
Shatara et al. (2000) Can. J. Physiol. Pharmacol. 78, 367-371
Shepherd et al. (1995) N. Engl. J. Med. 333, 1301-1307
Sorci-Thomas et al. (1990) J. Biol. Chem. 265, 2665-2670
Sorci-Thomas et al. (2000) J. Biol. Chem. 275, 12156-12163
Sparks et al. (1992) J. Biol. Chem. 267, 25839-25847
Sparrow et al. (1981) In: “Peptides: Synthesis-Structure-Function,” Roch and Gross, Eds., Pierce Chem. Co., Rockford, Ill. 253-256
Sparrow et al. (2002) J. Biol. Chem. 277, 10021-10027
Srinivas et al. (1990) Virology, 176, 48-57
Stein and Stein (1999) Atherosclerosis, 144, 285-303
Steiner et al. (1987) Circulation, 75, 124-130
Steinmetz and Utermann (1985) J. Biol. Chem. 260, 2258-2264
Sviridov et al. (1996) Biochemistry, 35, 189-196
Sviridov et al. (2000) J. Biol. Chem. 275, 19707-19712
Sviridov et al. (2000) J. Lipid Res. 41, 1872-1882
Swinkels et al. (1989) Arteriosclerosis, 9, 604-613
Tall and Wang (2000) J. Clin. Invest. 106, 1205-1207
Tall et al. (2000) Arterioscler. Thromb, Vasc. Biol. 20, 1185-1188
Tall et al. (2001) J. Clin. Invest. 108, 1273-1275
Tall et al. (2001) J. Clin. Invest. 108, 1273-1275
Tangirala et al. (1999) Circulation, 100, 1816-1822
Temel et al. (2002) J. Biol. Chem. 277, 26565-26572
The BIP study group (2000) Circulation, 102, 21-27
The International Task Force for Prevention of Coronary Heart Disease (1998) Nutr Metab Cardiovasc Dis. 8, 205-271
Thuahnai et al. (2001) J. Biol. Chem. 276, 43801-43808
Tribble et al. (1992) Atherosclerosis, 93, 189-199
Trigatti et al. (1999) Proc. Natl. Acad. Sci. USA, 96, 9322-9327
Tu et al. (1993) J. Biol. Chem. 268, 23098-23105
Utermann et al. (1984) Eur. J. Biochem. 144, 325-331
Vakkilainen et al. (2002) J. Lipid Res. 43, 598-603
van Eck et al. (2002) Proc. Natl. Acad. Sci. US.A., 99, 6298-6303
Venkatachalapathi et al. (1993) Proteins, 15, 349-359
von Eckardstein A. (1996) Curr Opin Lipidol. 7, 308-319
von Eckardstein and Assmann (2000) Curr Opin Lipidol. 11, 627-637
von Eckardstein et al. (1995) Arterioscler. Thromb. Vasc. Biol. 15, 690-701
von Eckardstein et al. (1996) Biochim. Biophys. Acta, 1301, 255-262
von Eckardstein et al. (2001) Arterioscl. Thromb. Vasc. Biol. 21, 13
Webb et al. (2002) J. Lipid Res. 43, 1890-1898
Whayne et al. (1981) Atherosclerosis, 39, 411-424
Yamashita et al. (1991) Metabolism, 40, 756-763
Yamazaki et al. (1983) J. Biol. Chem. 258, 5847-5853
Zhong et al. (1994) Peptide Research, 7(2): 99-106 (1984) JAMA, 251, 365-374

What is claimed is:

1. A mediator of reverse cholesterol transport, comprising the structure: 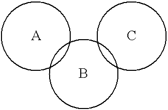
wherein A, B, and C may be in any order, and wherein:
A comprises an acidic moiety, comprising an acidic group or a bioisostere thereof;
B comprises an aromatic or lipophilic moiety comprising at least a portion of HMG CoA reductase inhibitor or analog thereof; and
C comprises a basic moiety, comprising a basic group or a bioisostere thereof.

2. The mediator of claim 1, wherein at least one of the alpha amino or alpha carboxy groups have been removed from their respective amino or carboxy terminal moieties.

3. The mediator of claim 1 or 2, wherein if not removed, the alpha amino group is capped with a protecting group selected from the group consisting of formyl, acetyl, phenylacetyl, benzoyl, pivolyl, 9-fluorenylmethyloxycarbonyl, 2-napthylic acid, nicotinic acid, a CH₃—CH₂)ₙ—CO— where n ranges from 1 to 20, di-tert-butyl-4-hydroxy-phenyl, naphthyl, substituted naphthyl, Fmoc, biphenyl, substituted phenyl, substituted heterocycles, alkyl, aryl, substituted aryl, cycloalkyl, fused cycloalkyl, saturated heteroaryl, and substituted saturated heteroaryl.

4. The mediator of claim 1 or 2, wherein if not removed, the alpha carboxy group is capped with a protecting group selected from the group consisting of an amine, such as RNH2 where R═H, di-tert-butyl-4-hydroxy-phenyl, naphthyl, substituted naphthyl, Fmoc, biphenyl, substituted phenyl, substituted heterocycles, alkyl, aryl, substituted aryl, cycloalkyl, fused cycloalkyl, saturated heteroaryl, and substituted saturated heteroaryl.

5. The mediator of claim 1, wherein the bioisostere of the acidic group is selected from the group consisting of:

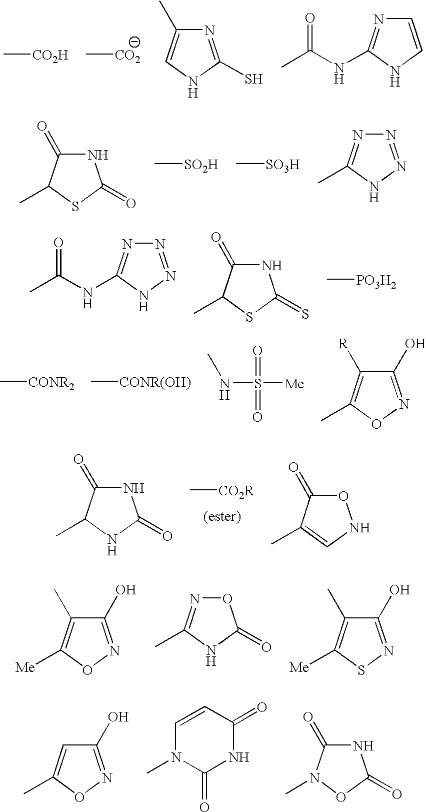

6. The mediator of claim 1, wherein the bioisostere of the basic group is selected from the group consisting of:

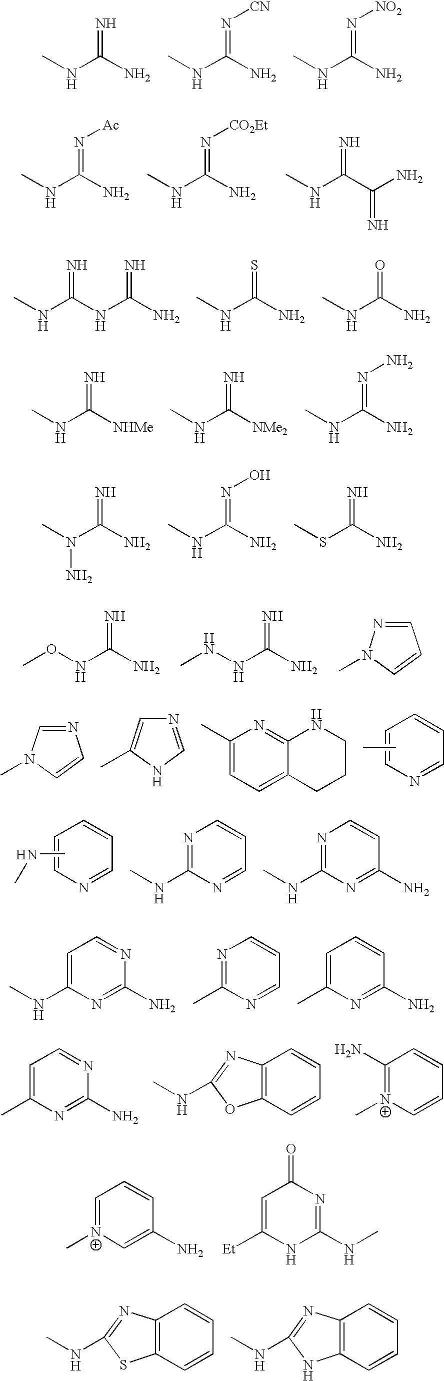

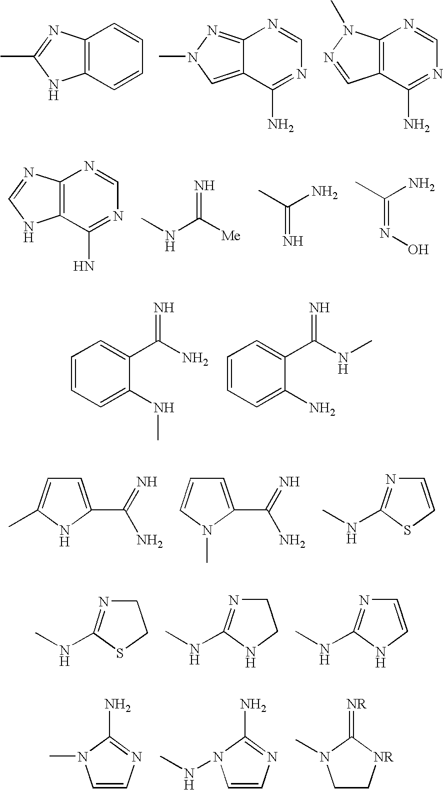

7. The mediator of claim 1, wherein the mediator is selected from the group consisting of:

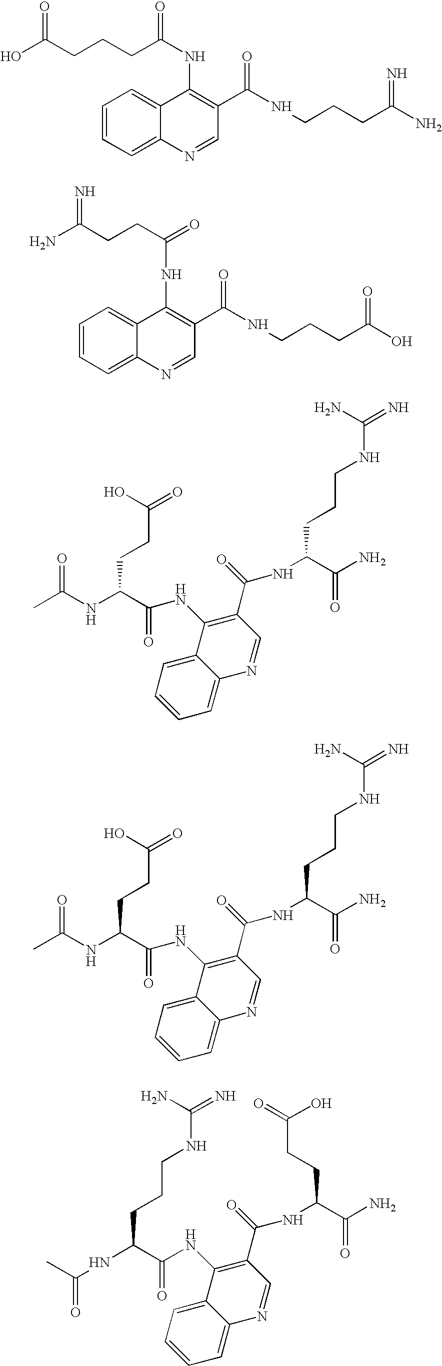

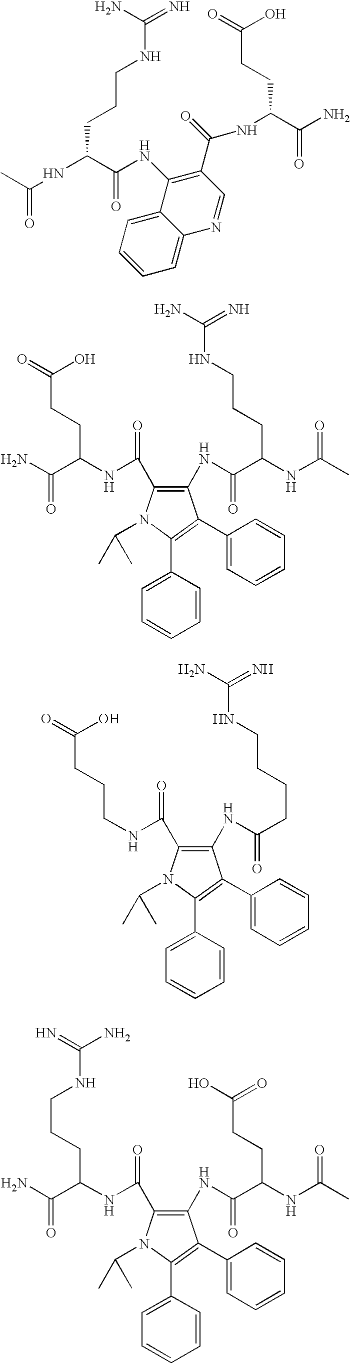

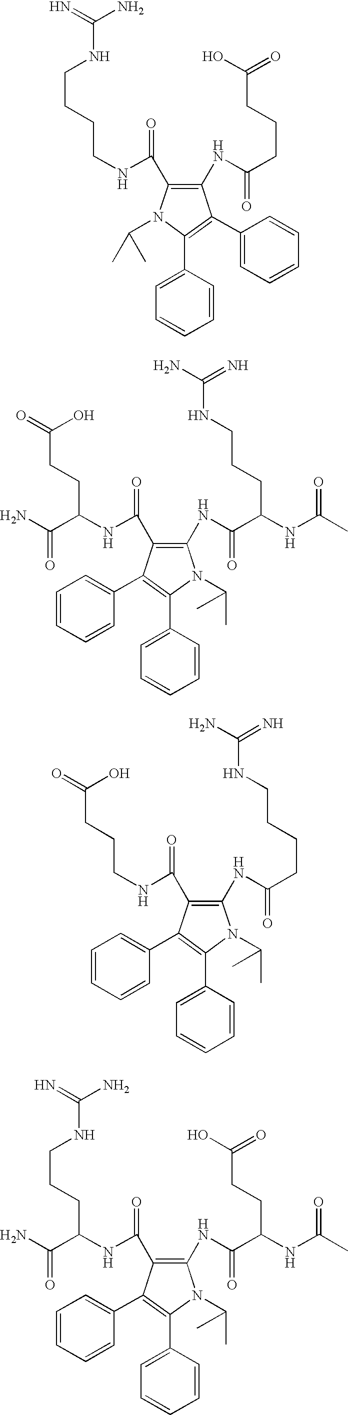

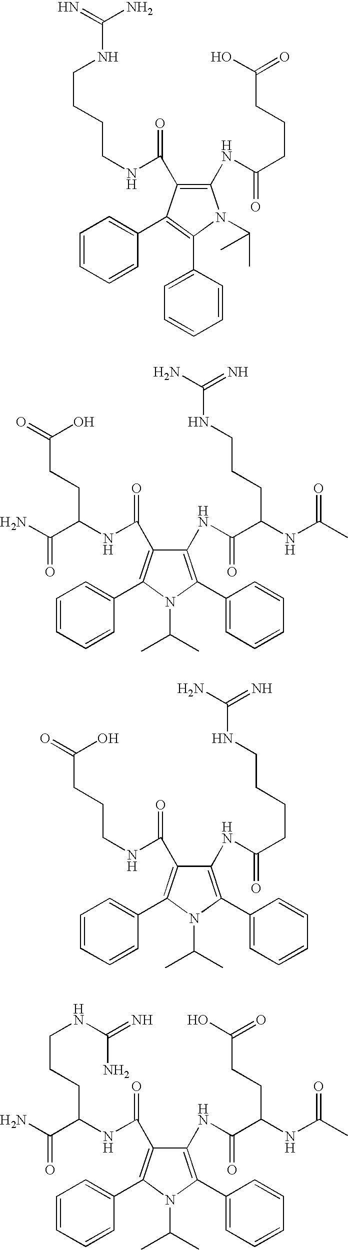

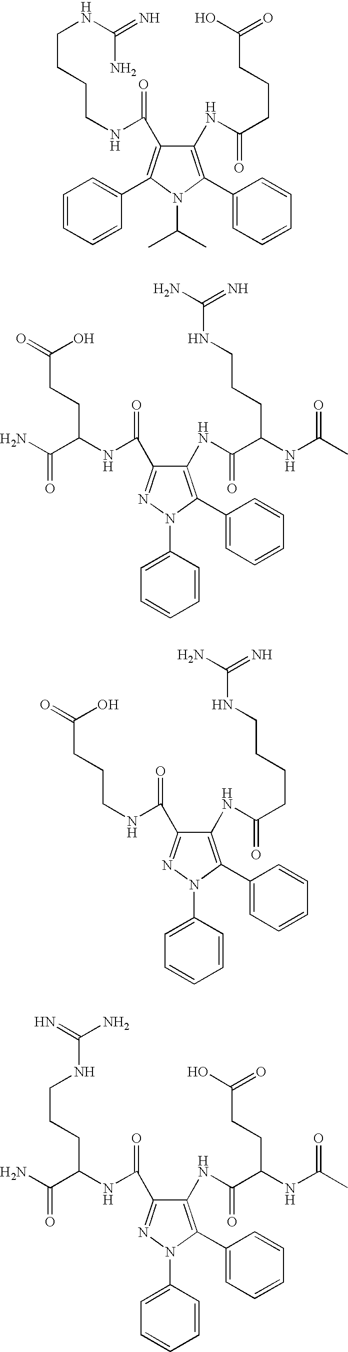

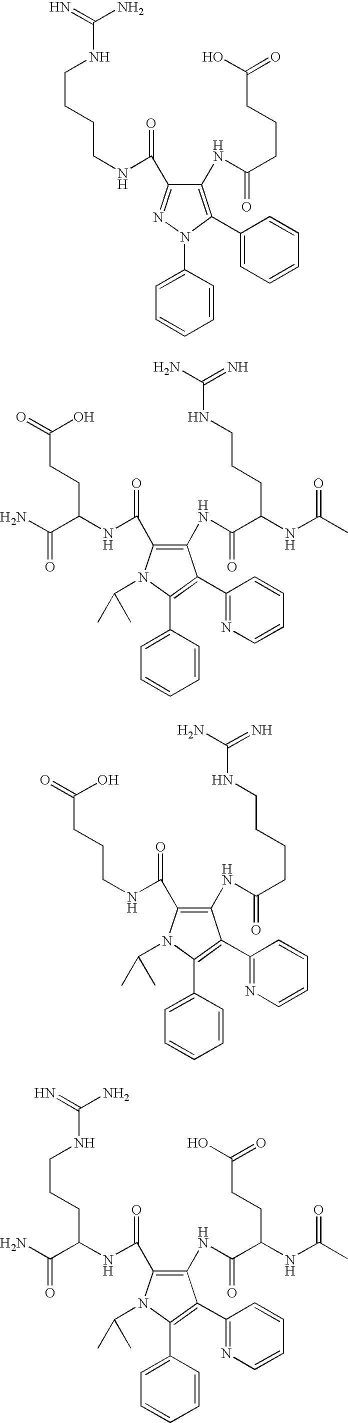

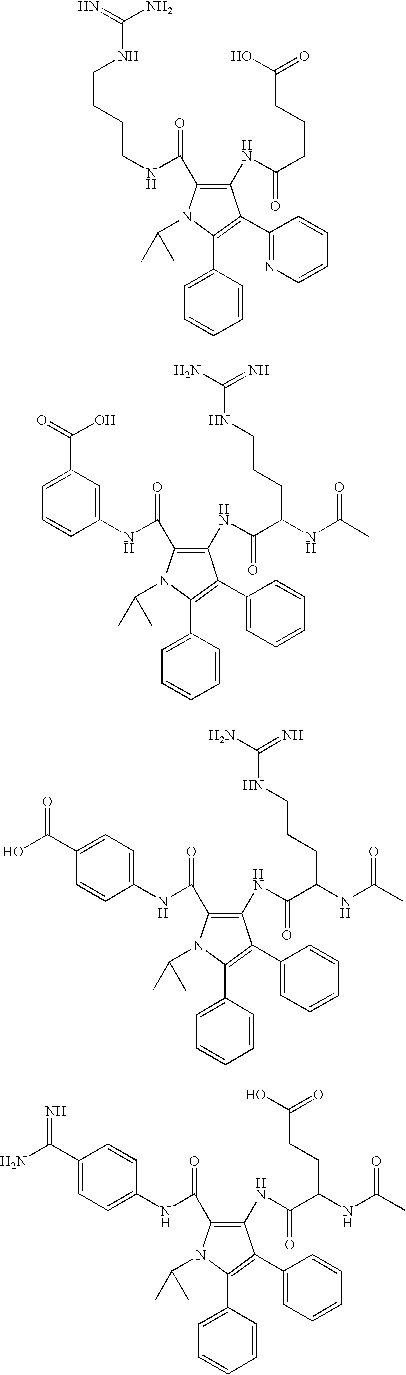

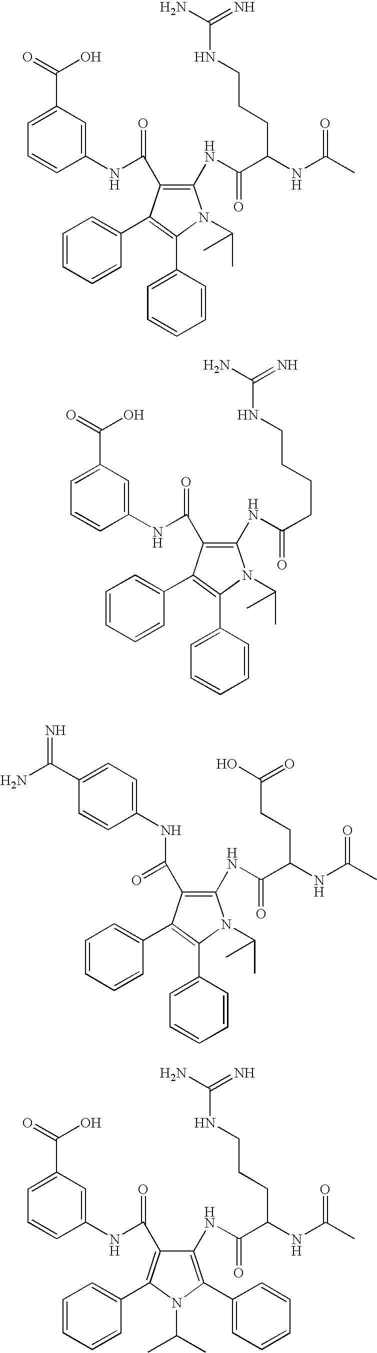

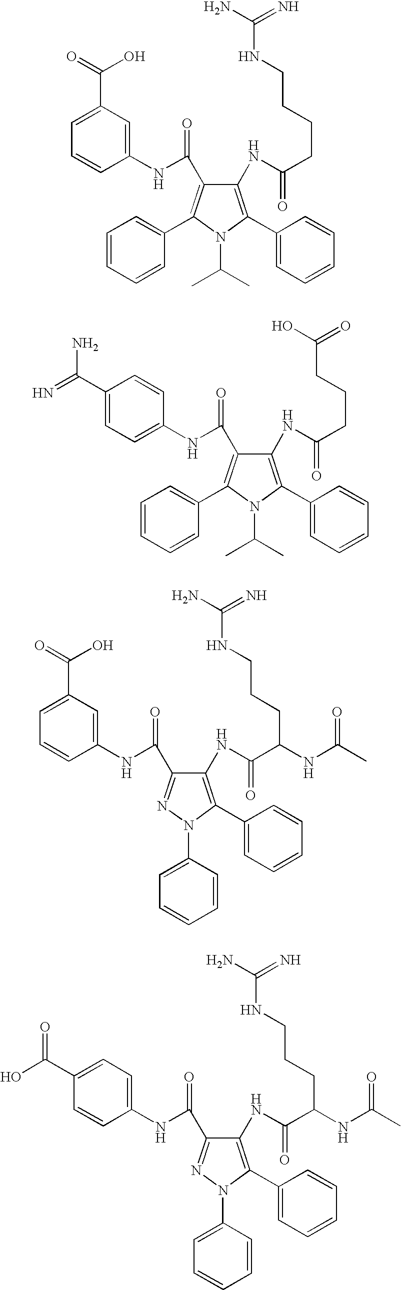

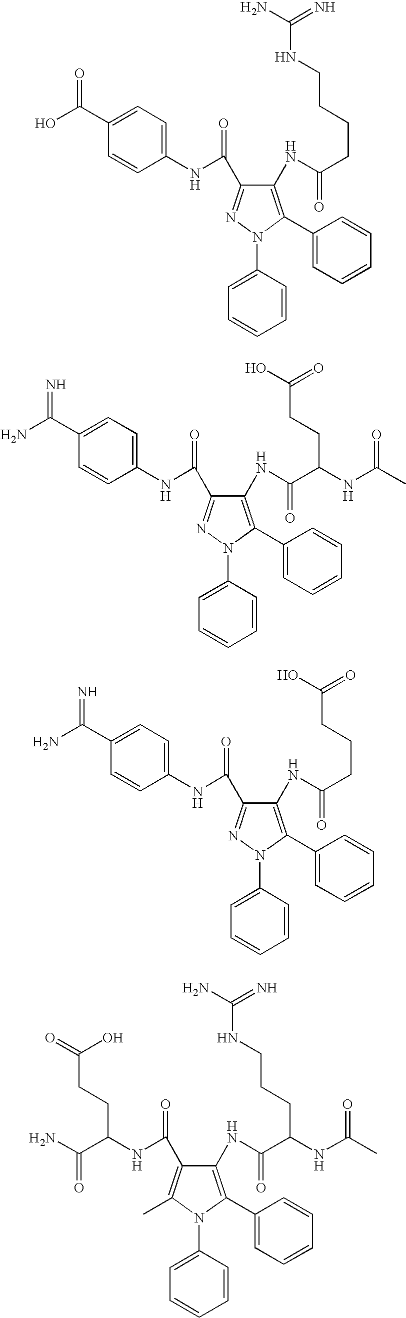

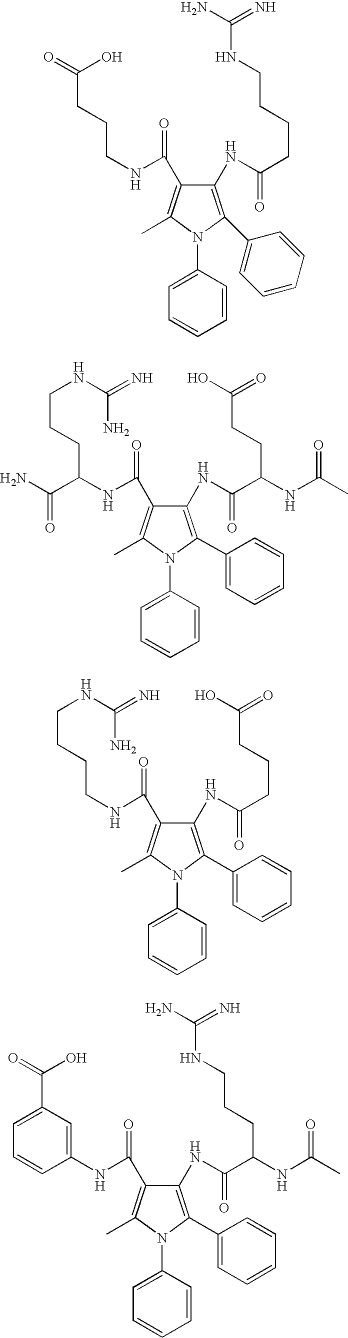

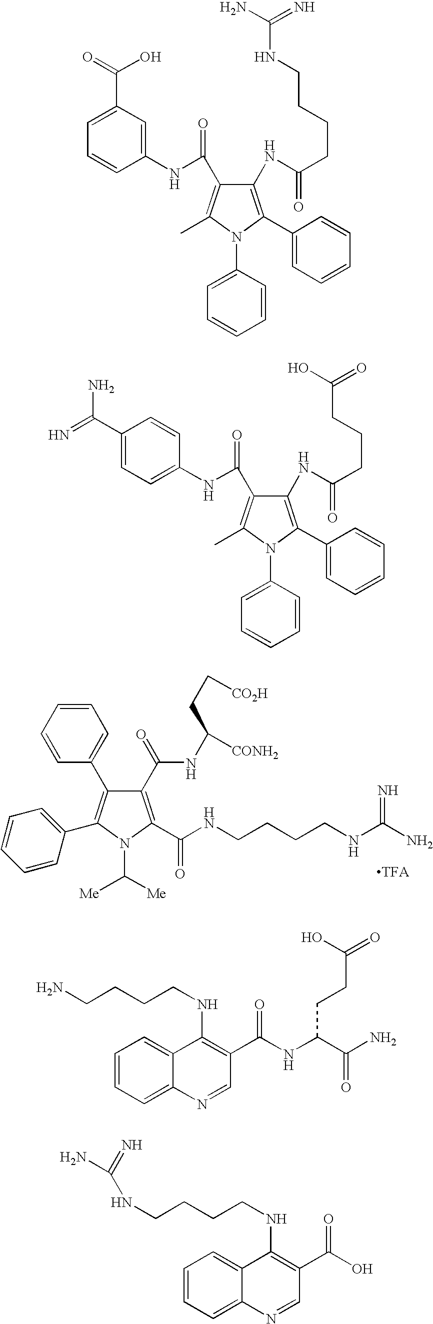

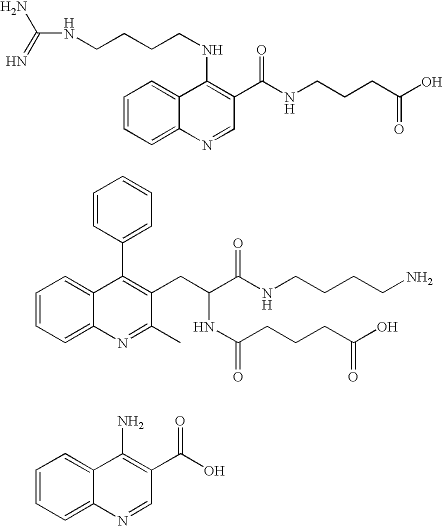

8. The compound 4-Agmatine-3-amidoGABAquinoline.

9. The compound 4-(1-(4-aminobutylcarbamoyl)-2-(2-methyl-4-phenylquinolin-3-yl)ethylcarbamoyl)butanoic acid

10. The compound:

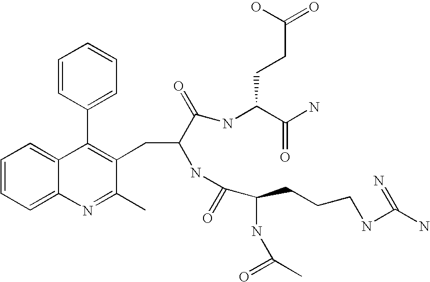

11. The compound 4-(5-guanidinopentylamino)quinoline-3-carboxylic acid.
